(19) United States
(12) Patent Application Publication
(10) Pub. No.: US 2005/0163711 A1
(43) Pub. Date: Jul. 28, 2005

(54) Intra-dermal delivery of biologically active agents

(75) Inventors: Colleen M. Nycz, Raleigh, NC (US); Glenn P. Vonk, Fuquay Varina, NC (US); John M. Brittingham, Wake Forest, NC (US); Ronald J. Pettis, Cary, NC (US); Alfred J. Harvey, Durham, NC (US); Robert L. Campbell, Bahama, NC (US); John A. Mikszta, Durham, NC (US); Diane E. Sutter, Cary, NC (US)

Correspondence Address: JONES DAY, 222 EAST 41ST ST, NEW YORK, NY, 10017, US

(73) Assignee: Becton, Dickinson and Company, Inc.

(21) Appl. No.: 10/867,908

(22) Filed: Jun. 14, 2004

Related U.S. Application Data
(60) Provisional application No. 60/538,473, filed on Jan. 26, 2004.
(60) Provisional application No. 60/502,225, filed on Sep. 12, 2003.
(60) Provisional application No. 60/477,950, filed on Jun. 13, 2003.
(60) Provisional application No. 60/489,920, filed on Jul. 25, 2003.

(51) Int. Cl. A61K 49/00

(52) U.S. Cl. 424/9.1

(57) ABSTRACT

The present invention relates to methods and devices for delivering one or more biologically active agents, particularly a diagnostic agent to the intradermal compartment of a subject's skin. The present invention provides an improved method of delivery of biologically active agents in that it provides among other benefits, rapid uptake into the local lymphatics, improved targeting to a particular tissue, improved bioavailability, improved tissue bioavailability, improved tissue specific kinetics, improved deposition of a pre-selected volume of the agent to be administered, and rapid biological and pharmacodynamics and biological and pharmacokinetics. This invention provides methods for rapid transport of agents through lymphatic vasculature accessed by intradermal delivery of the agent. Methods of the invention are particularly useful for delivery of diagnostic agents.

This application claims priority to U.S. Provisional Application Nos. 60/538,473 filed on Jan. 26, 2004; 60/502,225 filed on Sep. 12, 2003; 60/477,950 filed on Jun. 13, 2003; and 60/489,920 filed on Jul. 25, 2003; each of which is incorporated herein by reference in its entireties.

### 1. FIELD OF THE INVENTION

The present invention relates to methods and devices for delivering one or more biologically active agents, particularly a diagnostic agent to the intradermal compartment of a subject's skin. The present invention provides an improved method of delivery of biologically active agents in that it provides among other benefits, rapid uptake into the local lymphatics, improved targeting to a particular tissue, improved bioavailability, improved tissue bioavailability, improved tissue specific kinetics, improved deposition of a pre-selected volume of the agent to be administered, and rapid biological and pharmacodynamics and biological and pharmacokinetics. This invention provides methods for rapid transport of agents through lymphatic vasculature accessed by intradermal delivery of the agent. Methods of the invention are particularly useful for delivery of diagnostic agents.

### 2. BACKGROUND OF THE INVENTION

2.1 Delivery of Agents to the Skin

The importance of efficiently and safely administering pharmaceutical agents such as diagnostic agents and drugs has long been recognized. Difficulties associated with ensuring adequate bioavailability and reproducible absorption of large molecules, such as proteins that have arisen out of the biotechnology industry, have been recently highlighted (Cleland et al., Curr. Opin. Biotechnol. 12: 212-219, 2001). The use of conventional needles has long provided one approach for delivering pharmaceutical agents to humans and animals by administration through the skin. In general, injection avoids harsh conditions associated with oral delivery that commonly mitigate the desired effects of most biological therapies. Injection may also provide faster therapeutic effect than oral administration. Considerable effort has been made to achieve reproducible and efficacious delivery needle-based injection while improving the ease of use and reducing patient apprehension and/or pain associated with conventional needles. Furthermore, certain transcutaneous delivery systems eliminate needles entirely, and rely upon simple hydrophobic adsorption, chemical mediators or external driving forces such as iontophoretic currents or electroporation or thermal poration or sonophoresis to breach the statum corneum (the outermost layer of the skin) and deliver agents through the surface of the skin. However, such delivery systems do not, in general, reproducibly traverse the skin barriers or deliver pharmaceutical agents to a given depth below the surface of the skin. Consequently, clinical results can be variable. Thus, mechanical breach of the stratum corneum, such as with needles, is believed to provide the most reproducible method of administration of agents through the surface of the skin, and provides control and reliability in the placement of the administered agents.

Approaches for delivering agents beneath the surface of the skin have almost exclusively involved transdermal injections or infusions, i.e. delivery of agents through the skin to a site beneath the skin. Transdermal injections and infusions include subcutaneous, intramuscular or intravenous routes of administration of which, intramuscular (IM) and subcutaneous (SC) injections have been the most commonly used.

Anatomically, the outer surface of the body is made up of two major tissue layers, an outer epidermis and an underlying dermis, which together constitute the skin (for review, see Physiology, Biochemistry, and Molecular Biology of the Skin, Second Edition, L. A. Goldsmith, Ed., Oxford University Press, New York, 1991). The epidermis is subdivided into five layers or strata of a total thickness of between 75 and 150 μm. Beneath the epidermis lies the dermis, which contains two layers, an outermost portion referred to as the papillary dermis and a deeper layer referred to as the reticular dermis. The papillary dermis contains vast microcirculatory blood and lymphatic plexuses. In contrast, the reticular dermis is relatively acellular and avascular and made up of dense collagenous and elastic connective tissue. Beneath the epidermis and dermis is the subcutaneous tissue, also referred to as the hypodermis, which is composed of connective tissue and fatty tissue. Muscle tissue lies beneath the subcutaneous tissue.

As noted above, both the subcutaneous tissue and muscle tissue have been commonly used as sites for administration of pharmaceutical agents, including diagnostic agents. The dermis, however, has rarely been targeted as a site for administration of agents, and this may be due, at least in part, to the difficulty of precise needle placement into the intradermal compartment. Furthermore, even though the dermis, in particular, the papillary dermis has been known to have a high degree of vascularity, it has not heretofore been appreciated that one could take advantage of this high degree of vascularity to obtain an improved absorption profile for administered agents compared to subcutaneous administration.

One approach to administration beneath the surface to the skin and into the region of the intradermal compartment has been routinely used in the Mantoux tuberculin test. In this procedure, a purified protein derivative is injected at a shallow angle to the skin surface using a 27 or 30 gauge needle (Flynn et al., Chest 106: 1463-5, 1994). A degree of uncertainty in placement of the injection can, however, result in some false negative test results. Moreover, the test has involved a localized injection to elicit a response at the site of injection and the Mantoux approach has not led to the use of intradermal injection for systemic administration of agents.

Some groups have reported on systemic administration by what has been characterized as “intradermal” injection. In one such report, a comparison study of subcutaneous and what was described as “intradermal” injection was performed (Autret et al., Therapie 46:5-8, 1991). The pharmaceutical agent tested was calcitonin, a protein of a molecular weight of about 3600. Although it was stated that the drug was injected intradermally, the injections used a 4 mm needle pushed up to the base at an angle of 60°. This would have resulted in placement of the injectate at a depth of about 3.5 mm and into the lower portion of the reticular dermis or into the subcutaneous tissue rather than into the vascularized papillary dermis. If, in fact, this group injected into the lower portion of the reticular dermis rather than into the subcutaneous tissue, it would be expected that the agent would either be slowly absorbed in the relatively less vascular reticular dermis or diffuse into the subcutaneous region to result in what would be functionally the same as subcutaneous administration and absorption. Such actual or functional subcutaneous administration would explain the reported lack of difference between subcutaneous and what was characterized as intradermal administration, in the times at which maximum plasma concentration was reached, the concentrations at each assay time and the areas under the curves.

Similarly, Bressolle et al., administered sodium ceftazidime in what was characterized as “intradermal” injection using a 4 mm needle (Bressolle et al., J. Pharm. Sci. 82:1175-1178, 1993). This would have resulted in injection to a depth of 4 mm below the skin surface to produce actual or functional subcutaneous injection, although good subcutaneous absorption would have been anticipated in this instance because sodium ceftazidime is hydrophilic and of relatively low molecular weight.

Another group reported on what was described as intradermal drug delivery device (U.S. Pat. No. 5,007,501). Injection was indicated to be at a slow rate and the injection site was intended to be in some region below the epidermis, i.e., the interface between the epidermis and the dermis or the interior of the dermis or subcutaneous tissue. This reference, however, provided no teachings that would suggest a selective administration into the dermis nor did the reference suggest any possible pharmacokinetic advantage that might result from such selective administration.

Thus, there remains a continuing need for efficient and safe methods and devices for administration of pharmaceutical agents, especially diagnostic agents.

2.2 Delivery of Diagnostic Agents for Diagnosis of Diseases

Cancer is one of the most significant chronic conditions of the 20^{th} century. The American Cancer Society's Cancer Facts and Figures, 2003 indicates over 1.3 million Americans will receive a cancer diagnosis this year. In the US, cancer is second only to heart disease in mortality accounting for one of four deaths. In 2002, the National Institutes of Health estimated total costs of cancer totaled $171.6 billion with $61 billion in direct expenditures. Incidence of cancer is widely expected to increase as the US population ages further augmenting the impact of this condition. The current treatment regimens of chemotherapy and radiation essentially established in the 1970s and 1980s, have not changed dramatically. These treatments have limited utility since they are relatively nonspecific affecting processes in both normal and cancer cells. Another reason for the continued slow progress in treating cancer is that it arises primarily as a result of a breakdown in regulation at the molecular and cellular level. Although scientific understanding of cell regulatory processes is accelerating, the benefits of this knowledge are critically dependent on early detection and profiling of cancer at the cellular and molecular level in the clinic.

Many efforts have been focused on improving the detection of cancer. One recent advance in identifying cancer and its spread is the Sentinel Lymph Node Biopsy and Mapping procedure. Generally, this surgical procedure identifies the lymphatic network that drains the area in and around a tumor. Mapping this network allows the surgeon to visualize the patient's lymphatic system, aiding in the detection of cancerous growths and determining the lymphatic involvement in the disease. Diseased tissue and involved lymph nodes can be removed with greater efficiency and accuracy. The placement and number of involved lymph nodes affects subsequent treatment decisions. This is especially important for breast cancer patients. The sentinel mapping procedure employs intradermal delivery of a radioisotope-labeled tracer and a dye. The dye provides the visual enhancement while the tracer assists in identifying the sentinel lymph nodes that first drain from the tumor tissue. The tissue and nodes, once removed, are quickly evaluated by a waiting pathologist who examines the nodes and makes gross evaluations concerning cancer involvement. For the most part, macrometastasis can be identified, while micrometastasis requires a more lengthy examination post surgery. Together, the surgeon and pathologist decide how much additional tissue, as well as how many of the lymph nodes, are to be removed.

One problem with the current Sentinel Node Biopsy and Mapping procedure is its lack of sensitivity and specificity. Identification of cancer invasion into the lymph node is done by gross observation. Micrometastasis cannot be detected during the procedure. The reagents used are non-specific and do not aid in identifying rare cells. Addition of specific reagents in this manner improves sensitivity by giving the histologist and surgeon a more specific and sensitive signal that will allow for identification of rare cells in the tissue. Intradermal delivery of these reagents has been developed and used to substitute subcutaneous delivery, because intradermal delivery eliminates background signal from the tissue surrounding the lymph nodes. The current manual intradermal delivery works for reducing the background signal due to dye in non-lymphatic tissues. Despite obvious advantages, the skill and experience required to reliably perform sentinel node biopsies is a significant barrier to widespread clinical use. Infectious diseases similarly account for significant morbidity and mortality. For example, the CDC estimates 42 million people are infected with HIV worldwide. Present diagnostic methods generally rely on in vitro assay for diagnostic profiling. However, information regarding disease loci is, therefore lost. This information is of potential import for staging and therapy selection.

The present invention describes a novel method for profiling a disease, including infections using specific detection agents.

### 3. SUMMARY OF THE INVENTION

The present invention provides a method for administering one or more biologically active agents, preferably a diagnostic agent, to a subject's skin, in which the biologically active agent is delivered to the intradermal (ID) compartment of the subject's skin. The present invention is based, in part, on the unexpected discovery by the inventors that when such agents are delivered to the ID compartment, they are transported to the local lymphatic system rapidly as compared to conventional modes of delivery, including subcutaneous delivery and ID Mantoux delivery, and thus provide the benefits disclosed herein. Although not intending to be bound by a particular mechanism of action, agents delivered in accordance with the methods of the invention are transported in vivo through the local lymphatic system, excreted into the systemic blood circulation and into deeper tissue environments. The agent is then degraded or metabolized by, for example, the liver, kidneys, or spleen. Although not intending to be bound by a particular mechanism of action, it is the biomechanical manipulation of the extracellular matrix (ECM) through the precise delivery of agents in the intradermal compartment that enables rapid uptake into the local lymphatics and lymph nodes by the methods described herein.

The present invention provides an improved method of delivery of biologically active agents, in that it provides among other benefits, rapid uptake into the local lymphatics, improved targeting to a particular tissue, i.e., improved deposition of the delivered agent into the particular tissue, i.e., group or layer of cells that together perform a specific function, improved systemic bioavailability, improved tissue bioavailability, improved deposition of a pre-selected volume of the agent to be administered, improved tissue-specific kinetics (i.e., includes improved or altered biological pharmacodynamics and biological pharmacokinetics) rapid biological and pharmaco-dynamics (PD), and rapid biological and pharmacokinetics (PK). Such benefits of the invention are improved over other methods of delivering biologically active agents which deposit the agent into deeper tissue compartments than the intradermal compartment including for example subcutaneous compartment and intramuscular compartment. Such benefits of the methods of the invention are especially useful for the delivery of diagnostic agents. Intradermal delivery of a diagnostic agent in accordance with the methods of the invention deposits the diagnostic agent into the intradermal and lymphatic compartments, thus creating a rapid and biologically significant concentration of the diagnostic agent in these compartments. Rapid diagnostics can therefore be performed using less diagnostic agent with significant advantages as outlined herein.

Intradermally delivered biologically active agents have improved tissue bioavailability in a particular tissue, including but not limited to, skin tissue, lymphatic tissue (e.g., lymph nodes), mucosal tissue, reproductive tissue, cervical tissue, vaginal tissue and any part of the body that consists of different types of tissue and that performs a particular function, i.e., an organ, including but not limited to lung, spleen, colon, thymus. In some embodiments, the tissue includes any tissue that interacts with or is accessible to the environment, e.g., skin, mucosal tissue. The invention encompasses any tissue or organ with a mucosal layer. Other tissues encompassed by the invention include without limitation Haemolymphoid System; Lymphoid Tissue (e.g., Epithelium-associated lymphoid Tissue and Mucosa-associated lymphoid Tissue or MALT (MALT can be further divided as organized mucosa-associated lymphoid Tissue (O-MALT) and diffused lymphoid tissue (D-MALT)); primary Lymphoid Tissue (e.g., thymus and bone marrow); Secondary Lymphoid Tissue (e.g., lymph node, spleen, alimentary, respiratory and Urigenital). It will be appreciated by one skilled in the art that MALT secondary includes gut associated lymphoid tissue (GALT); Bronchial associated lymphoid tissue (BALT), and genitourinary system. MALT specifically comprises lymph nodes, spleen, tissue associated with epithelial surfaces such as palentine and nasopharyngeal tonsils, Peyer's Patches in the small intestine and various nodules in the respiratory and urinogenital systems, the skin and conjunctivia of the eye. O-MALT includes the peripharyngeal lymphoid ring of the tonsils (palentine, lingual, nasopharyngeal and tubal), Oesophageal nodules and similar lymphoid tissue scattered throughout the alimentary tract from the duuuodenum to the anal canal. The delivery of a biologically active agent in accordance with the methods of the invention results in improved tissue bioavailability as compared to when the same agent is delivered to the subcutaneous (SC) compartment or when the same agent is delivered by the intradermal (ID) Mantoux method. Improved tissue bioavailability of agents delivered in accordance with the methods of the invention is particularly useful when delivering diagnostic agents to the ID compartment, as it reduces the amount of the diagnostic agent required for each diagnostic procedure, which may be difficult and costly to obtain. The reduced amount of the diagnostic agent further reduces the likelihood of side effects associated with the diagnostic procedure, e.g., toxicity.

Intradermally delivered biologically active agents have improved tissue bioavailability in a particular tissue compared to when the same agent is delivered to a deeper tissue compartment such as the SC compartment and the IM compartment. The improved tissue bioavailability of the agents delivered in accordance with the methods of the invention can be determined using methods and parameters known to those skilled in the art, for example, by measuring the total amount of the agent accumulated in a particular tissue using, for example, histological methods known to those skilled in the art and disclosed herein. Alternatively, improved tissue bioavailability of the agents can be assessed as the amount of the agent presented to the particular tissue, the amount of the agent accumulated per mass or volume of a particular tissue, amount of the agent accumulated per unit time in a particular mass or volume of a particular tissue.

Biologically active agents delivered in accordance with the methods of the invention are deposited in the intradermal compartment and first distributed with high bioavailability to the lymphatic tissue local to the administration site, followed by a more wide spread lymphatic delivery in to the general circulation. In some embodiments, the methods of the present invention are particularly effective for diagnosis of a disease, disorder, or infection in deeper tissues, e.g., in vivo detection of an infection in an organ or tissue such as lung or inflammation of an organ or tissue such as appendix or joints.

Intradermally delivered biologically active agents, especially diagnostic agents, exhibit more rapid onset and clearance versus conventional delivery including SC delivery and ID Mantoux delivery. The methods of the invention thus confer several advantages when delivering a diagnostic agent to the ID compartment of a subject's skin. First, the methods disclosed herein reduce potential side effects and discomfort due to the diagnostic procedures. Second, they enable the rapid and repeated trial of sequential procedures in a single diagnostic session. Third, they reduce the time required in expensive medical or imaging facilities. Fourth, they facilitate real time studies of physiology. Fifth, they reduce potential background signal generated by unbound and un-cleared diagnostic reagents. Sixth, patients experience reduced pain from the methods of the invention in comparison to pain perceived from IV administration, SC injection, Mantoux injection, or surgical biopsy.

Delivering biologically active agents, including diagnostic agents in accordance with the methods of the invention is preferred over traditional modes of delivery including SC delivery and ID Mantoux delivery because the amount of the pre-selected dose of the agent deposited in the lymphatic tissue is increased, as measured, for example, using histopathological methods or other methods known to one skilled in the art, such as Fluorescence Activated Cell Sorting (FACS) and imaging methods disclosed herein.

As used herein, delivery to the intradermal compartment or intradermally delivered is intended to mean administration of a biologically active agent into the dermis in such a manner that the agent readily reaches the richly vascularized papillary dermis and is rapidly absorbed into the blood capillaries and/or lymphatic vessels to become systemically bioavailable. Such can result from placement of the agent in the upper region of the dermis, i.e., the papillary dermis or in the upper portion of the relatively less vascular reticular dermis such that the agent readily diffuses into the papillary dermis. The controlled delivery of a biologically active agent in this dermal compartment below the papillary dermis in the reticular dermis, but sufficiently above the interface between the dermis and the subcutaneous tissue, should enable an efficient (outward) migration of the agent to the (undisturbed) vascular and lymphatic microcapillary bed (in the papillary dermis), where it can be absorbed into circulation via these microcapillaries without being sequestered in transit by any other cutaneous tissue compartment. In some embodiments, placement of a biologically active agent predominately at a depth of at least about 0.3 mm, more preferably, at least about 0.4 mm and most preferably at least about 0.5 mm up to a depth of no more than about 2.5 mm, more preferably, no more than about 2.0 mm and most preferably no more than about 1.7 mm will result in rapid absorption of the agent. Although not intending to be bound by a particular mechanism of action, placement of the biologically active agent predominately at greater depths and/or into the lower portion of the reticular dermis may result in less effective uptake of the agent by the lymphatics, as the agent will be slowly absorbed in the less vascular reticular dermis or in the subcutaneous compartment.

Biologically active agents, including diagnostic agents delivered in accordance with the methods of the invention will achieve higher maximum concentrations of the agents and allow reduced overall dosing. Therefore, the dose can be reduced, providing an economic benefit, as well as a physiological benefit since lesser amounts of the drug or diagnostic agent has to be cleared by the body.

Another benefit of the invention is no change in systemic elimination rates or intrinsic clearance mechanisms of biologically active agents, including diagnostic agents. This indicates this dosing route has no change in the biological mechanism for systemic clearance. This is an advantage from a regulatory standpoint, since degradation and clearance pathways need not be reinvestigated prior to filing for FDA approval. This is also beneficial from a pharmacokinetics standpoint, since it allows predictability of dosing regimes. Some agents may be eliminated from the body more rapidly if their clearance mechanism are concentration dependent. Since ID delivery results in higher Cₘₐₓ, clearance rate may be altered, although the intrinsic mechanism remains unchanged.

The improved benefits associated with ID delivery of biologically active agents in accordance with the methods of the invention can be achieved using not only microdevice-based injection systems, but other delivery systems such as needle-less or needle-free ballistic injection of fluids or powders into the ID compartment, enhanced ionotophoresis through microdevices, and direct deposition of fluid, solids, or other dosing forms into the skin. In specific embodiments, the administration of the biologically active agent is accomplished through insertion of a needle or cannula into the intradermal compartment of the subject's skin.

The intradermal delivery of diagnostic agents in accordance with the present invention are particularly beneficial in the diagnosis of diseases, including chronic and acute diseases, which include, but are not limited to, lymphoma, leukemia, breast cancer, melanoma, colorectal cancer, head and neck cancer, lung cancer, cancer metastasis, including micrometastasis, viral infections, e.g., HIV, RSV, immune disorders such as rejection, metabolic disorders, diseases or disorders of the lymphatic system, any disease affecting the lymph nodes, and infectious diseases. Chronic diseases according to the U.S. National Center for Health Statistics refers to a disease or disorder which lasts for three months or longer. Although not intending to be bound by a particular mechanism of action, diagnostic agents delivered in accordance with the methods of the invention are deposited in the intradermal compartment and taken up by the lymphatic system, where its recognition and binding of a particular cell in a particular tissue indicate the presence of a cell or disease state. The present invention is useful for diagnostic procedures including, but not limited to, surgical methods, biopsies, non-invasive screening and imaging and image-guided biopsies.

The present invention provides improved methods for diagnosis and/or detection of a disease, e.g., cancer, by improving sensitivity, the amount of the agent deposited, tissue bioavailability, faster onset and clearance of the delivered diagnostic agent. The invention provides a method for administration of at least one diagnostic agent for the detection of a disease, particularly cancer, comprising delivering the agent into the ID compartment of a subject's skin at a controlled rate, volume and pressure so that the agent is deposited into the ID compartment and taken up by the lymphatic vasculature.

The methods of the invention are particularly improved over conventional cancer detection procedures for the detection of a tumor sentinal node, e.g., breast tumor sentinal node, or a lymph node that drains the tumor in a human subject, because more than 75% of the pre-selected volume of the diagnostic agent is deposited into the intradermal compartment, relative to when the same pre-selected volume is delivered to the intradermal compartment by the traditional methods of delivery of such agents, e.g., ID Mantoux method.

The present invention provides improved methods for current sentinel node biopsy procedure and mapping surgical procedure by improving the uptake and the bioavailability of the diagnostic agents to the local lymphatic system. The invention provides a method for administration of at least one diagnostic agent for the detection of a tumor sentinal node, e.g., breast tumor sentinal node, or a lymph node that drains the tumor in a human subject, comprising delivering the agent into the intradermal compartment of the human subject's skin so that the agent is transported to the local lymphatic system. In other embodiments, the invention provides a method for administration of at least one diagnostic agent for the detection of a tumor sentinal node, e.g., breast tumor sentinal node, or a lymph node that drains the tumor in a human subject, comprising delivering the agent into the intradermal compartment of the human subject's skin so that the agent has a higher tissue bioavailability compared to when the same agent is delivered by the ID Mantoux method. In yet other specific embodiments, the invention provides a method for administration of at least one diagnostic agent for the detection of a tumor sentinal node, e.g., breast tumor sentinal node, or a lymph node that drains the tumor in a human subject, comprising delivering the agent into the intradermal compartment of the human subject's skin so that the agent has a faster onset and clearance compared to when the same agent is delivered by the ID Mantoux method.

The methods of the instant invention provide improved prognostic methods using specific agents (versus non-specific agents) to assess therapeutic efficacy of a treatment regimen of a disease, for example, by monitoring cellular genetic profiles in assessing gene regulation and expression over time. Traditionally, in vitro analysis of cellular genetic profiles have been used to assess gene regulation and expression over time as a tool in assessing therapeutic efficacy. Such in vitro methods have numerous shortcomings including, but not limited to, inaccuracies, the removal of cells from the body can cause the destruction of RNA and DNA thereby altering the genetic profile in the specimen, information about the morphological locus of the genetic lesion is potentially lost using ex-vivo methods, and cell differentiation and regulation may be influenced by removal from the extracellular environment in vivo. By using the methods of the present invention, intradermal administration of specific diagnostic agents capable of associating and/or binding a specific marker for a disease provides for assessment of disease as it exists in the patient. Thus, the methods taught by the present invention influence the choices of therapy available to the practitioner.

The methods of the invention are particularly useful for methods of integrated diagnosis and therapy. Accurate diagnosis of a disease is largely an unmet need for example in oncology, where few diagnostic agents indicate which therapeutic choices will succeed with any reliability. The methods of the invention provide improved methods for integrated diagnosis and therapy by administration of formulations comprising one or more diagnostic agents in combination with one or more therapeutic agents. The present invention provides methods to target diagnostic agents and therapeutic agents to a particular cell in a particular tissue. In a specific embodiment, the invention encompasses delivering formulations comprising one or more diagnostic agents in combination with one or more therapeutic agents to the ID compartment of a subject's skin such that a specific action of the diagnostic agent triggers an action, e.g., biological effect, of the therapeutic agent. The combination of targeted diagnostic delivery with targeted therapeutics delivery in accordance with the methods of the invention provides for enhanced patient care. This embodiment teaches the advantages of combining intradermal therapeutic delivery with diagnostic agents. The combination of delivering a diagnostic and a therapeutic agent to the ID compartment provides a powerful tool for improving the treatment of a disease in a subject.

3.1 Definitions

As used herein, “intradermal” refers to administration of a biologically active agent into the dermis in such a manner that the agent readily reaches the richly vascularized papillary dermis and is rapidly absorbed into the blood capillaries and/or lymphatic vessels to become systemically bioavailable. Such can result from placement of the agent in the upper region of the dermis, i.e., the papillary dermis or in the upper portion of the relatively less vascular reticular dermis such that the agent readily diffuses into the papillary dermis. The controlled delivery of a biologically active agent in this dermal compartment below the papillary dermis in the reticular dermis, but sufficiently above the interface between the dermis and the subcutaneous tissue, should enable an efficient (outward) migration of the agent to the (undisturbed) vascular and lymphatic microcapillary bed (in the papillary dermis), where it can be absorbed into systemic circulation via these microcapillaries without being sequestered in transit by any other cutaneous tissue compartment. In some embodiments, placement of a biologically active agent predominately at a depth of at least about 0.3 mm, more preferably, at least about 0.4 mm and most preferably at least about 0.5 mm up to a depth of no more than about 2.5 mm, more preferably, no more than about 2.0 mm and most preferably no more than about 1.7 mm will result in rapid absorption the agent. Although not intending to be bound by a particular mechanism of action, placement of the biologically active agent predominately at greater depths and/or into the lower portion of the reticular dermis or the SC compartment which results in less effective uptake by the lymphatics.

As used herein, “intradermal delivery” means the delivery of agents to the intradermal compartment as described by Pettis et al. in WO 02/02179 A1 (PCT/US01/20782) and U.S. application Ser. No. 09/606,909; each of which is incorporated herein by reference in their entireties.

As used herein, “ID Mantoux delivery” refers to the traditional ID Mantoux tuberculin test where an agent is injected at a shallow angle to the skin surface using a 27 or 30 gauge needle and standard syringe (see, e.g., Flynn et al., Chest 106: 1463-5, 1994, which is incorporated herein by reference in its entirety). The Mantoux technique involves inserting the needle into the skin laterally, then “snaking” the needle further into the ID tissue. The technique is known to be quite difficult to perform and requires specialized training. A degree of imprecision in placement of the delivery results in a significant number of false negative test results. Moreover, the method involves a localized injection to elicit a response at the site of injection and the Mantoux approach has not led to the use of intradermal injection for systemic administration of agents. When delivering the agent by ID Mantoux, the needle is substantially parallel to the surface at the skin, preferably at an angle of no more that 30° and best described as being between 10° and 15°. Mantoux deposition of injectate, when performed correctly, results in an elliptical pattern with the injectate in the SC and ID tissues. ID deposition as described herein results in a rounded deposition pattern of the injectate contained in the ID tissue. When delivering an agent by the ID Mantoux method, the insertion angle of the needle is preferably at a 15° angle parallel to the skin surface. Histological examination of the injection site after an agent has been administered by ID Mantoux results in an elliptical wheal deposition pattern, and a substantial part of the agent delivered gets deposited into the SC compartment of the skin rather than the ID compartment. ID Mantoux method is typically used clinically in diagnostic procedures such as sentinal node biopsy procedures for detection of tumors, however the method is quite difficult to perform and requires specialized training and has numerous limitations including, sites of administration, complications of injection, and patient discomfort.

As used herein subcutaneous delivery refers to deposition of an agent into the subcutaneous layer of a subject's skin at a depth greater than 2.5 mm.

As used herein, “pharmacokinetics, pharmacodynamics and bioavailability” are as described by Pettis et al. in WO 02/02179 A1 (PCT/US01/20782 having a priority date of Jun. 29, 2000).

As used herein, “improved pharmacokinetics” means increased bioavailability, decreased lag time (T_{tag}), decreased Tₘₐₓ, more rapid absorption rates, more rapid onset and/or increased Cₘₐₓ for a given amount of agent administered, compared to conventional administration methods.

As used herein, “bioavailability”, means the total amount of a given dosage of the administered agent that reaches the blood compartment. This is generally measured as the area under the curve in a plot of concentration vs. time.

As used herein, “lag time” means the delay between the administration of the agent and time to measurable or detectable blood or plasma levels. Tₘₐₓ is a value representing the time to achieve maximal blood concentration of the agent, and Cₘₐₓ is the maximum blood concentration reached with a given dose and administration method. The time for onset is a function of T_{tag}, Tₘₐₓ and Cₘₐₓ, as all of these parameters influence the time necessary to achieve a blood (or target tissue) concentration necessary to realize a biological effect. Tₘₐₓ and Cₘₐₓ can be determined by visual inspection of graphical results and can often provide sufficient information to compare two methods of administration of a agent. However, numerical values can be determined more precisely by kinetic analysis using mathematical models and/or other means known to those of skill in the art.

As used herein, the term “particles” includes any formed element comprising monomers, polymers, lipids, amphiphiles, fatty acids, steroids, proteins, and other materials known to aggregate, self-assemble or which can be processed into particles. Particles also include unilamelar, multilamelar, random tortuous path and solid morphologies. Representative examples include liposomes, microcrystalline materials, particulate MRI contrast agents, polymeric beads (i.e. latex and HEMA), but most preferably hollow particles, such as microbubbles, useful for ultrasonic imaging.

As used herein “tissue” refers to a group or layer of cells that together perform a function including but not limited to, skin tissue, lymphatic tissue (e.g., lymph nodes), mucosal tissue, reproductive tissue, cervical tissue, vaginal tissue and any part of the body that consists of different types of tissue and that performs a particular function, i.e., an organ, including but not limited to lung, spleen, colon, thymus. As used herein, tissue includes any tissue that interacts with or is accessible to the environment, e.g., skin, mucosal tissue.

As used herein, “tissue-bioavailability” means the amount of an agent that is biologically available in vivo in a particular tissue. These amounts are commonly measured as activities that may relate to binding, labeling, detection, transport, stability, biological effect, or other measurable properties useful for diagnosis and/or therapy. In addition, it is understood that the definition of “tissue-bioavailability” also includes the amount of an agent available for use in a particular tissue. “Tissue-bioavailability” includes the total amount of the agent accumulated in a particular tissue, the amount of the agent presented to the particular tissue, the amount of the agent accumulated per mass/volume of particular tissue, and amount of the agent accumulated per unit time in a particular mass/volume of the particular tissue. Tissue bioavailability includes the amount of an agent that is available in vivo in a particular tissue or a collection of tissues such as those that make up the vasculature and/or various organs of the body (i.e., a part of the body that consists of different types of tissue and that performs a particular function. Examples include the spleen, thymus, lung, lymph nodes, heart and brain).

As used herein, “conventional delivery” means any method for delivering any material that has, or is thought to have, improved biological kinetics and biological dynamics similar to, or slower than, subcutaneous delivery. Conventional delivery may include subcutaneous, iontophoretic, and intradermal delivery methods such as those described in U.S. Pat. No. 5,800,420 to Gross.

As used herein a “biological entity” includes but is not limited to a cell, group or collection of cells, a bacteria, a virus, a pathogen, a protein, a plaque, and a parasitic agent.

As used herein, “targeted delivery” means the use of intradermal delivery to particular specific tissues and/or organs and/or a biological entity not otherwise accessed or understood to be accessed by the conventional delivery methods.

Targeted tissues include, but are not limited to, the intradermal compartment near the site of administration and the local lymphatic structures including initial lymphatics, lymphatic vessels, ducts and lymph nodes. Targeted tissues also include but are not limited to, skin tissue, lymphatic tissue (e.g., lymph nodes), mucosal tissue, reproductive tissue, cervical tissue, vaginal tissue and any part of the body that consists of different types of tissue and that performs a particular function, i.e., an organ, including but not limited to lung, spleen, colon, thymus. and any tissue that interacts with or is accessible to the environment, e.g., skin, mucosal tissue.

As used herein, a “specific agent” includes such compounds as proteins, immunoglobulins (e.g., multi-specific Igs, single chaing Igs, Ig fragments, polyclonal antibodies and their fragments, monoclonal antibodies and their fragments), peptides (e.g., peptide receptors, selectins), binding proteins (maltose binding protein, glucose-galactose binding protein)), Nucleotides, Nucleic Acids (e.g., PNAs, RNAs, modified RNA/DNA, aptamers), Receptors (e.g., Acetylcholine receptor), Enzymes (e.g., Glucose Osicase, HIV Protease and reverse transcriptase), Carbohydrates (e.g., NCAMs, Sialic acids), Cells (e.g., Insulin & Glucose responsive cells), bacteriophage (e.g., filamentous phage), viruses (e.g., HIV), chemospecific agents (e.g., cyptands, crown ethers, boronates).

As used herein, “chemospecific agent” means a chemically synthesized molecule that binds specifically to a bio-molecule. Examples of chemospecific agents include, but are not limited to, PNAs such as GeneGRIP™ as commercialized by Gene Therapy Systems Inc., photoaptamers as commercialized by SomaLogic, sialic acid binders as described by Shinkai, S, et. al. J. A. Chem. Soc. 2001, 123. 10239-10244, Wang et al., Current Organic Chemistry 2002, 6, 1285-1317, Striegler, S. Current Organic Chemistry 2003, 7, 81-102, Wang, et. al., Bioorganic & Medicinal Chemistry Letters 2002, 2175-2177, and boronic acids for detection of carbohydrates as described in U.S. 2002/0143475 (Colorimetric and Fluorometric analysis of Carbohydrates). All of the above mentioned references are incorporated herein by reference in their entireties.

As used herein, a “non-specific agent” includes such compounds as dyes, dye conjugates, radionuclides, or formed elements such as liposomes, colloids or latex particles.

As used herein, “marker” means any receptor, molecule or other chemical or biological entity that is specifically found in tissue that it is desired to identify, in particular tissue affected by a disease or disorder (e.g. a metastases). Where an antibody is used as the tracer, the marker is an antigen. Examples of antigen markers include CD4, CD8, CD90 and other antigenic markers mentioned herein, as well as those that are known in the art. Non-limiting examples of such markers include: proteins or receptors such as Her2/neu or epidermal growth factor receptor (EGFR) for breast cancer, melastatin for melanoma, CD22 for lymphoma, and HIV protease for HIV infection. Markers may also be carbohydrates such as sialic acids for metastases or NCAMs for neuroendocrine disease or cancer, cells that are glucose or insulin responsive for diabetes, viruses or bacteriophage for HIV or infectious diseases, nucleotides or nucleic acids such as aptamers for genetic profiling detection of disease or detection of disease molecular level. An example of such a disease is Diffuse Large B Cell lymphoma.

As used herein, the terms “disorder” and “disease” are used interchangeably to refer to a condition in a subject. Diseases include to any interruption, cessation, or disorder of body functions, systems or organs.

As used herein, the term “cancer” refers to a neoplasm or tumor resulting from abnormal uncontrolled growth of cells. As used herein, cancer explicitly includes, leukemias and lymphomas. The term “cancer” refers to a disease involving cells that have the potential to metastasize to distal sites and exhibit phenotypic traits that differ from those of non-cancer cells, for example, formation of colonies in a three-dimensional substrate such as soft agar or the formation of tubular networks or weblike matrices in a three-dimensional basement membrane or extracellular matrix preparation. Non-cancer cells do not form colonies in soft agar and form distinct sphere-like structures in three-dimensional basement membrane or extracellular matrix preparations. Cancer cells acquire a characteristic set of functional capabilities during their development, albeit through various mechanisms. Such capabilities include evading apoptosis, self-sufficiency in growth signals, insensitivity to anti-growth signals, tissue invasion/metastasis, limitless explicative potential, and sustained angiogenesis. The term “cancer cell” is meant to encompass both pre-malignant and malignant cancer cells. In some embodiments, cancer refers to a benign tumor, which has remained localized. In other embodiments, cancer refers to a malignant tumor, which has invaded and destroyed neighboring body structures and spread to distant sites. In yet other embodiments, the cancer is associated with a specific cancer antigen.

As used herein, the terms “subject” and “patient” are used interchangeably. As used herein, a subject is preferably a mammal such as a non-primate (e.g., cows, pigs, horses, cats, dogs, rats etc.) and a primate (e.g., monkey and human), most preferably a human.

### 4. DESCRIPTION OF THE FIGURES

FIG. 1 MOUSE LYMPH NODES a diagram depicting the location of draining lymph nodes in the mouse.

FIGS. 2A-E ID DELIVERY TO LYMPHATICS

A. shows highly stained superficial inguinal lymph nodes in the mouse 1 hour post intradermal delivery of 1% Evans Blue solution by the method of the present invention.

B: shows Intra-dermal (ID) vs. Subcutaneous (SC) Injection of Evans Blue Dye in Yorkshire Swine. Diagram of swine depicting location of injection sites.

C. ID and SC injections. Arrow indicates location of SC injection.

D. ID and SC injections post mortem.

E. ID and SC injection site resection. Note the trafficking of the Evans Blue dye from the ID injection site to the inguinal node and depoting of the dye at the SC injection site.

FIGS. 3A and B. ID DELIVERY TO LYMPHATICS

A. shows the percentage of cells positive for CD90 and CD4 or CD8 or CD19 in the draining lymph node over time.

B. shows flow cytometry plots of labeled cell suspension from lymph nodes of naive, 30 minutes, and 1-hour post anti-CD90-FITC antibody injection mice (n=2).

FIGS. 4A-C. ID DELIVERY TO LYMPHATICS

A. shows in vivo fluorescent staining of lymph tissue with injected antibody sections at 1 hour post FITC-antibody injection under 40 times magnification.

B shows H and E staining of the cells at 1 hour post FITC-antibody injection under 40 times magnification.

C shows the overlay of 4a and 4b.

FIG. 5 ID DELIVERY PROFILE shows the path of the biologically active agent after being intradermally delivered, by the method of the present invention.

FIG. 6 IN VIVO TARGETED DIAGNOSTICS shows a diagram of potential targets for delivery in the lymphatic system.

FIG. 7A AND B ID IN VIVO TARGETED DIAGNOSTICS. shows comparative time profiles for ID and SC (SubQ) delivery of labeled antibody to mouse lymph nodes.

A. Delivery Method. Comparison of ID and SC delivery to Lymph Nodes

B. Enhanced Detection of Lymphatic cells using ID Delivery Time profile of antibody labeled cells in mouse lymph nodes

FIG. 8 IN VIVO TARGETED DIAGNOSTICS-APPLICATION shows a diagram of how the method may be applied to a breast tumor, and a demonstration of T-cell labeling in mouse lymph node.

FIG. 9 shows results of injection of 50 ul EB through a 34G, 1.0 mm needle at a rate of 45 uL/min in a Yorkshire pig. The circled areas within the reticular dermis, separate from the main injection depot, show cross-sections of the draining lymphatic vessels (blue).

FIGS. 10 AND 11 show results of injection of 100 uL of EB through a 34G, 1.0 mm needle at a rate of 45 uL/min. in a Yorkshire pig.

FIGS. 12 AND 13 show results of injection at two sites interdermally in the flank of a Yorkshire pig with 100 uL of EB through a 34 G, 1.0 mm needle at a rate of 100 uL/min.

FIG. 14 shows results of injection intradermally in the flank of a Yorkshire pig with 100 uL of EB through a 34G, 1.5 mm needle at a rate of 100 uL/min.

FIG. 15 shows an example of lymphatic vessels (blue) from a 2 mm injection. Both a cross-section and a length-wise section can be seen in the circled area.

FIG. 16 shows an example of lymphatic vessels (blue) trafficking the intradermally injected Evans Blue dye from the site of injection to the inguinal lymph nodes. Insert shows close-up of resected inguinal lymph node.

FIGS. 17A-C shows the Number of Injected Fluorescent Beads present in the Inguinal Lymph Node Over Time. Comparison of Intra-dermal and Subcutaneous Injection.

A. 50 nm sized beads

B. 1 μm sized beads

C. 10 μm sized beads.

FIGS. 18A-B PERCENT OF CD8 POSITIVE T CELLS, IN MOUSE SPLEENS. Graphs depicting the Percent of CD8 Positive T Cells, in mouse spleens, labeled with CD90-FITC antibody over time. CD90-FITC antibody was either ID or SC injected into mice and spleens were monitored for cell-associated signal.

FIG. 19. IMAGING OF SWINE ABDOMINAL BLOOD VASCULATURE AFTER 12.5 mg IV INJECTION OF ICG. Right Inguinal node location depicted in box. Only blood vasculature is illuminated and not the lymph nodes. Imaging continued episodically for 30 minutes post injection without illumination of lymph nodes.

FIGS. 20A-C. DOSE SPARING-IV AND MICRONEEDLE ID INJECTION. Imaging of Lymphatic vasculature and inguinal node of swine immediately following injection of ICG using a 34G, 1 mm depth, microneedle.

A. Three injections on swine abdomen (left side), top injection 200 uls of 80 ug/ml ICG, bottom 2 injections 75 uls of 80 ug/ml ICG.

B. Imaging of lymphatic vasculature and left inguinal lymph node of swine immediately after top injection from A.

C. Imaging of lymphatic vasculature and right inguinal nodes after 2 separate injections of 80 ug/ml ICG on right hind leg. Note the individual lymphatic vasculature from each injection feeding separately into the nodes.

FIGS. 21A-D. DEMONSTRATION OF ICG DYE TRAFFICKING SPEED. ICG injected ID using 34G, 1 mm depth, microneedle. Injection performed above left mammary chain of swine. ICG travel calculated to be 7 cm/sec for this injection.

FIG. 22 NEEDLE DEVICE. An exploded, perspective illustration of a needle assembly designed according to this invention.

FIG. 23 NEEDLE DEVICE. A partial cross-sectional illustration of the embodiment in FIG. 22.

FIG. 24 NEEDLE DEVICE. Embodiment of FIG. 22 attached to a syringe body to form an injection device.

FIG. 25 ID INJECTION TECHNIQUE. A perspective view of one technique for making an ID injection

FIG. 26 ID INJECTION TECHNIQUE. A perspective view of a second technique for making an ID injection.

FIG. 27 ID INJECTION TECHNIQUE. A perspective view of a third technique for making an ID injection.

FIG. 28 ID INJECTION TECHNIQUE. A perspective view of a fourth technique for making an ID injection.

FIG. 29A AND B IDELIVERY OF CARDIO GREEN IMAGING AGENT

A. INJECTIONS ON RIGHT HIND LEG AND LEFT SIDE MAMMARY CHAIN. Left and right inguinal nodes illuminated.

B. INJECTIONS ON RIGHT HIND LEG AND LEFT SIDE MAMMARY CHAIN. Inverted Image. Left and right inguinal nodes illuminated.

FIG. 30 COMPARISON OF MANTOUX AND ID DELIVERY. The photo of the mantoux injection clearly-shows the track of the needle (blue line through dermis leading to depot.) The majority of the EB was injected into the SC. The photo of the delivery with a 34G 1 mm needle shows that the injection was completely within the dermis. Drainage to the lymphatics can already be seen (circled).

FIGS. 31A and B. GRAPHS OF MAXIMUM AND AVERAGE SUSTAINED PRESSURE AS A FUNCTION OF INSERTION DEPTH FOR BOTH VENTRAL AND DORSAL SWINE INJECTIONS. Maximum pressure was the single highest pressure recorded during the first 100 seconds of infusion. Average sustained pressure was average for all pressure readings from 100 to 300 seconds. The maximum and average sustained pressures for each injection configuration were averaged together and plotted.

A. Average back pressure plotted as function of needle length. All infusions in the ventral region of the animal.

B. Average back pressure plotted as function of needle length. All infusions in the dorsal region of the animal.

FIG. 33. IN VIVO STAINING. Graph depicting the percent of T and B cells stained, in vivo, in the draining lymph nodes of mice.

### 5. DETAILED DESCRIPTION OF THE INVENTION

The present invention provides a method for administering one or more biologically active agents, preferably a diagnostic agent, to a subject's skin, in which the biologically active agent is delivered to the intradermal (ID) compartment of the subject's skin. The present invention is based, in part, on the unexpected discovery by the inventors that when such agents are delivered to the ID compartment, they are transported to the local lymphatic system rapidly compared to conventional modes of delivery, including subcutaneous delivery and ID Mantoux delivery, and thus provide the benefits disclosed herein. Although not intending to be bound by a particular mechanism of action, agents delivered in accordance with the methods of the invention are transported in vivo through the local lymphatic system, excreted into the systemic blood circulation and into deeper tissue environments. The agent is then degraded or metabolized by, for example, the liver, kidneys, or spleen. Although not intending to be bound by a particular mechanism of action, it is the biomechanical manipulation of the extracellular matrix (ECM) through the precise delivery of agents in the intradermal compartment that enables rapid uptake into the local lymphatics and lymph nodes by the method described herein.

The present invention provides an improved method of delivery of biologically active agents in that it provides among other benefits, rapid uptake into the local lymphatics, improved targeting to a particular tissue, i.e., improved deposition of the delivered agent into the particular tissue, i.e., group or layer of cells that together perform a specific function, improved systemic bioavailability, improved tissue bioavailability, improved deposition of a pre-selected volume of the agent to be administered, improved tissue-specific kinetics rapid biological and pharmaco-dynamics (PD), and rapid biological and pharmacokinetics (PK). Such benefits of the methods of the invention are especially useful for the delivery of diagnostic agents. Intradermal delivery of a diagnostic agent in accordance with the methods of the invention deposits the diagnostic agent into the intradermal and lymphatic compartments thus creating a rapid and biologically significant concentration of the diagnostic agent in these compartments. Rapid diagnostics can therefore be performed using less diagnostic agent with significant advantages as outlined herein.

Intradermally delivered biologically active agents have improved tissue bioavailability in a particular tissue, including but not limited to, skin tissue, lymphatic tissue (e.g., lymph nodes), mucosal tissue, reproductive tissue, cervical tissue, vaginal tissue and any part of the body that consists of different types of tissue and that performs a particular function, i.e., an organ, including but not limited to lung, spleen, colon, thymus. In some embodiments, the tissue includes any tissue that interacts with or is accessible to the environment, e.g., skin, mucosal tissue. Other tissue encompassed by the invention include without limitation Haemolymphoid System; Lymphoid Tissue (e.g., Epithelium-associated lymphoid Tissue and Mucosa-associated lymphoid Tissue or MALT (MALT can be further divided as organized mucosa-associated lymphoid Tissue (O-MALT) and diffused lymphoid tissue (D-MALT)); primary Lymphoid Tissue (e.g., thymus and bone marrow); Secondary Lymphoid Tissue (e.g., lymph node, spleen, alimentary, respiratory and Urigenital). It will be appreciated by one skilled in the art that MALT secondary includes gut associated lymphoid tissue (GALT); Bronchial associated lymphoid tissue (BALT), and genitourinary system. MALT specifically comprises lymph nodes, spleen, tissue associated with epithelial surfaces such as palentine and nasopharyngeal tonsils, Peyer's Patches in the small intestine and various nodules in the respiratory and urinogenital systems, the skin and conjunctivia of the eye. O-MALT includes the peripharyngeal lymphoid ring of the tonsils (palentine, lingual, nasopharyngeal and tubal), Oesophageal nodules and similar lymphoid tissue scattered throughout the alimentary tract from the duuuodenum to the anal canal. Intradermally delivered biologically active agents have improved tissue bioavailability in a particular tissue compared to when the same agent is delivered to a deeper tissue compartment such as the SC compartment and the IM compartment.

The delivery of a biologically active agent in accordance with the methods of the invention results in improved tissue bioavailability as compared to when the same agent is delivered to the subcutaneous (SC) compartment or when the same agent is delivered by the intradermal (ID) Mantoux method. The delivery of a biologically active agent in accordance with the methods of the invention results in improved tissue bioavailability as compared to when the same agent is delivered to a deeper tissue compartment, e.g., SC, IM. Improved tissue bioavailability of agents delivered in accordance with the methods of the invention is particularly useful when delivering diagnostic agents to the ID compartment, as it reduces the amount of the diagnostic agent required for each diagnostic procedure, which may be difficult and costly to obtain. The reduced amount of the diagnostic agent further reduces the likelihood of side effects associated with the diagnostic procedure, e.g., toxicity.

The improved tissue bioavailability of the agents delivered in accordance with the methods of the invention can be determined using methods and parameters known to those skilled in the art, for example by measuring the total amount of the agent accumulated in a particular tissue using, for example, histological methods known to those skilled in the art and disclosed herein. Alternatively improved tissue bioavailability of the agents can be assessed as the amount of the agent presented to the particular tissue, the amount of the agent accumulated per mass or volume of particular tissue, amount of the agent accumulated per unit time in a particular mass or volume of the particular tissue.

Biologically active agents delivered in accordance with the methods of the invention are deposited in the intradermal compartment and first distributed with high bioavailability to the lymphatic tissue local to the administration site, followed by a more wide spread lymphatic delivery in to the general circulation. In some embodiments, the methods of the present invention are particularly effective for diagnosis of a disease, disorder, or infection in deeper tissues, e.g., in vivo detection of an infection in an organ or tissue such as lung or inflammation of an organ or tissue such as appendix or joints.

Biologically active agents delivered in accordance with the methods of the invention show immediate transport and uptake within at least 5 minutes, at least 10 minutes, at least 15 minutes, preferably no more than within 20 minutes after the injection to the lymphatic system, as monitored visually in real time using common methods in the art (e.g., MRI, X-Ray, Ultrasound, CT, PET, SPECT, Optical (fluorescence, bioluminescence, chemiluminescence), photoacoustic, RAMAN and SERS imgaing) or in vitro using common methods in the art (e.g., histological examination, flow cytometry) and those disclosed herein, see, Section 6.2. Agents delivered in accordance with the methods of the invention are transported to the lymphatic system and deposited in a particular tissue with velocities of at least 10 cm per second, preferably at least 5-10 cm per second. It will be appreciated by one skilled in the art that the rate at which the agent is transported to the lymphatic system and deposited in a particular tissue depends on various parameters including but not limited to volume of injection, rate of injection, biochemical and physical characteristic of the agent, and site of injection.

In some embodiments, biologically active agents, including diagnostic agents delivered in accordance with the methods of the invention specifically recognize and bind a cell in a particular tissue in which they are deposited. In other embodiments, biologically active agents delivered in accordance with the methods of the invention are delivered to the ID compartment so that the amount of the pre-selected dose of the agent deposited in the target tissue is increased by at least 0.1% compared to when the agent is delivered outside of the intradermal space, e.g., subcutaneous compartment (SC), intramuscular compartment (IM). The invention contemplates that the amount of the pre-selected dose of the agent deposited in the target tissue is increased by at least 100%, at least 150%, at least 200%, at least 200%, at least 250%, preferably by at least 350% or 3.5×, up to 1750%, the amount achieved when the agent is administered by routes outside of the intradermal compartment, e.g., SC, IM and thus delivered to a deeper tissue compartment.

The invention encompasses methods of delivering the biologically active agents to the ID compartment so that the amount of the pre-selected dose of the agent deposited in the target tissue is increased by the amounts specified herein compared to when the agent is delivered outside of the intradermal space, e.g., subcutaneous compartment (SC), intramuscular compartment (IM) such that the increase in amount is detected as early as3 minutes post-injection, or as early as 3 hours post injection. Preferably the increase in deposition of the agent in the particular tissue may persist for at least 21 days, at least 27 days.

In some embodiments, the concentration of the biologically active agent deposited in a particular tissue after ID delivery is about 5 nanograms of the agent agent per 50 micrograms of the particular tissue; 10 picograms of the agent per 50 micrograms of the particular tissue; 29 nanograms of the agent per 50 micrograms of the particular tissue; 10 picograms of the agent per 50 micrograms of the particular tissue to about 29 nanograms of the agent per 50 micrograms of the particular tissue; 10 picograms of the agent per 50 micrograms of particular tissue to about 150 nanograms of the agent per 50 micrograms of the particular tissue.

In other embodiments, the concentration of the biologically active agent, e.g., a diagnostic agent, deposited in a particular tissue after ID delivery is about 10 pg to about 15 ug of the agent agent per 50 micrograms of the particular tissue, or about 1 cg to about 30 ng of the agent agent per 50 micrograms of the particular tissue.

Unlike subcutaneous delivery, intradermal methods, as described herein, enhance the biological kinetics, biological dynamics, and tissue bioavailability of the biologically active agents delivered, including diagnostic and therapeutic agents. Intradermal delivery of biologically active agents in accordance with the methods of the invention are taken up by the lymphatic system and deposited in a particular tissue without the need of “massaging” the injection site, which is unlike other conventional modes of delivery, including subcutaneous delivery. Biologically active agents delivered to the subcutaneous compartment do not achieve deposition in a target tissue and/or lymphatic transport unless the injection site is massaged to induce such transport of the delivered agent. Although not intending to be bound by a particular mechanism of action, delivery methods, such as intravenous injection, rely on dissemination of the agent of interest from the general circulation into the target tissue. Dissemination of the biologically active agent into the tissue is dependent on many variables and the bioavailability found in the general circulation is not always optimal for a given target tissue. The intravenous and subcutaneous methods for delivery of an agent are limiting especially when the target tissue is in the lymphatic system. In addition, intradermal delivery, as described by the present invention, offers an alternate transport mechanism in which a specific agent is presented to the intradermal compartment and flows to the general circulation via the lymphatic system and area capillaries. Although others have described intradermal delivery to lymphatic vasculature, none have defined specific conditions or devices for reliable access of these tissues. Although not intending to be bound by a particular mechanism of action, delivering biologically active agents (as liquids or suspensions) into the intradermal compartment in accordance with the methods of the invention results in increased interstitial pressure which, in turn, opens the lymphatic vasculature permitting high rates of sustained flow until fluid flow is terminated. The inventors have found that this lymphatic transport occurs surprisingly fast, permitting immediate access to the lymphatic vasculature and general circulation. Methods of the invention result in uptake of agents into the lymphatic system, rather than capillary uptake, thus resulting in the benefits disclosed herein including but not limited to enhanced rate and activity of targeting.

Biologically active agents delivered in accordance with the methods of the invention are deposited in the intradermal compartment and first distributed with high bioavailability to the lymphatic tissue local to the administration site, followed by a more wide spread lymphatic delivery in to the general circulation. In some embodiments, the methods of the present invention are particularly effective for diagnosis of a disease, disorder or infection in deeper tissues.

In some embodiments, the invention encompasses targeted intraderaml delivery of a biologically active agent to a particular biological entity including but not limited to a cell, a group or collection of cells, a bacteria (e.g., Escherichia coli, Klebsiella pneumoniae, Staphylococcus aureus, Enterococcus faecials, Candida albicans, Proteus vulgaris, Staphylococcus viridans, and Pseudomonas aeruginosa), a pathogen (e.g., B-lymphotropic papovavirus (LPV); Bordatella pertussis; Borna Disease virus (BDV); Bovine coronavirus; Choriomeningitis virus; Dengue virus; a virus, E. coli; Ebola; Echovirus 1; Echovirus-11 (EV); Endotoxin (LPS); Enteric bacteria; Enteric Orphan virus; Enteroviruses; Feline leukemia virus; Foot and mouth disease virus; Gibbon ape leukemia virus (GALV); Gram-negative bacteria; Heliobacter pylori; Hepatitis B virus (HBV); Herpes Simplex Virus; HIV-1; Human cytomegalovirus; Human coronovirus; Influenza A, B & C; Legionella; Leishmania mexicana; Listeria monocytogenes; Measles virus; Meningococcus; Morbilliviruses; Mouse hepatitis virus; Murine leukemia virus; Murine gamma herpes virus; Murine retrovirus; Murine coronavirus mouse hepatitis virus; Mycobacterium avium-M; Neisseria gonorrhoeae; Newcastle disease virus; Parvovirus B19; Plasmodium falciparum; Pox Virus; Pseudomonas; Rotavirus; Samonella typhiurium; Shigella; Streptococci; T-cell lymphotropic virus 1; Vaccinia virus); a plaque, and a parasitic agent. Once an agent is delivered to a biological entity in accordance with the methods of the invention, any of the detection, imaging methods known to one skilled in the art and disclosed herein can be used to detect and image the entity. The methods of the invention encompasse methods for delivering a biologically active agent where the agent specifically binds a biological entity.

Directly targeting the intradermal compartment as taught by the invention provides more rapid onset of effects of biologically active agents, including diagnostic agents, and higher bioavailability including, tissue bioavailability, relative to other modes of delivery of such agents, including subcutaneous and ID Mantoux delivery. The inventors have found that agents delivered in accordance with the methods of the invention can be rapidly absorbed and systemically distributed via controlled ID administration that selectively accesses the dermal vascular and lymphatic microcapillaries, thus the agents may exert their beneficial effects more rapidly than SC administration and ID Mantoux delivery. Additionally the inventors have found that delivering an agent into the ID compartment takes advantage of the dermal microcirculation and the interaction between hydrostatic and osmotic pressures, in the dermal extra-cellular matrix, and the lymphatic vessels. It is in the dermal interstitium that the blood and lymph systems interact in the skin. As blood travels to the smallest capillaries, plasma fluid and proteins are forced out into the interstitial compartment. Osmotic and biomechanical forces result in perfusion of the fluid through the interstitium and into the local initial lymphatics. The initial lymphatics are permeable to macromolecules and therefore act in maintaining osmotic and hydrostatic pressures within the tissue compartment. The typical flow rate of the lymphatics is 10-100 times less than the flow rate of blood. The lymphatic system consists of 5 major conduits. They include lymphatic capillaries and collecting vessels, found in the dermis, lymph nodes, trunks, and ducts. Lymph forms when interstitial fluid moves into the lymph capillaries. It then drains into the collecting vessels. The vessels pass through at least one but usually several lymph nodes clusters. The vessels leaving the nodes drain into larger trunks, which in turn lead into the ducts. The ducts return the lymph back to the bloodstream, completing the circuit (Swartz, M. A. 2001, Adv. Drug Del. Rev. 50: 3-20). The lymphatic system flow is uni-directional with the lymphatic capillaries as the initial fluid collection conduit. In the interstitium the lymphatic capillaries primary role is to maintain hydrostatic and osmotic pressure in the tissue. This is accomplished through the interaction of the capillary anchoring filaments and the extra-cellular matrix (ECM). As fluid fills the interstitium, tissue pressure increases and places stress on the ECM, essentially stretching the tissue, and the anchoring filaments holding the lymphatics in place are pulled with the ECM. This movement pulls the lymphatic capillary open allowing the fluid to flow rapidly from the tissue in order to re-establish appropriate hydrostatic and osmotic-pressures. Therefore, the mechanical integrity of the ECM plays an important role in the lymphatic function. Extensive or chronic degradation of the ECM eventually renders lymphatic vessels non-responsive to the changes in the interstitium and causes dysfunction (Swartz et al., 1999, J Biomech. 32(12):1297-307). Although not intending to be bound by a particular mechanism of action, it is the biomechanical manipulation of the ECM through the precise delivery of agents in the interstitium that enables rapid uptake into the local lymphatics and lymph nodes by the method described herein.

While tissue stress contributes greatly to the uptake of fluid additional factors contribute as well. These include the concentration, size, charge, and molecular weight of the pharmaceutical agent being delivered and the interplay between these characteristics and the surrounding intradermal tissue environment (Charman et al., 1992 Lymphatic Transport of Drugs, Boca Raton: CRC Press Inc.). Manipulation of these factors is contingent upon exact and reproducible access to the interstitium. Even though the dermis, in particular, the papillary dermis has been known to have a high degree of vascularity, it has not heretofore been appreciated that one could take advantage of the high degree of vascularity as well as the interaction between the ECM and the lymphatic vasculature to obtain an improved absorption profile for administered agents compared to alternative routes of transdermal administration.

Intradermally delivered biologically agents, especially diagnostic agents, exhibit more rapid onset and clearance versus conventional delivery including SC delivery and ID mantoux delivery. These properties confer several advantages when delivering a diagnostic agent to the ID compartment of a subject's skin. First, it reduces potential side effects and discomfort due to the diagnostic procedures. Second, it enables the rapid and repeated trial of sequential procedures in a single diagnostic session. Third, it reduces the time required in expensive medical or imaging facilities. Fourth, it facilitates real time studies of physiology. Fifth, it reduces potential background signal generated by unbound and un-cleared diagnostic reagents. Sixth, patients experience reduced pain versus IV administration, SC injection or surgical biopsy.

Delivering biologically active agents, including diagnostic agents in accordance with the methods of the invention is preferred over traditional modes of delivery including SC delivery and ID Mantoux delivery because the amount of the pre-selected dose of the agent deposited in the lymphatic tissue is increased, as-measured for example using histopathological methods or other methods known to one skilled in the art such as flow cytomety (FACS) or imaging.

As used herein, delivery to the intradermal compartment or intradermally delivered is intended to mean administration of a biologically active agent into the dermis in such a manner that the agent readily reaches the richly vascularized papillary dermis and is rapidly absorbed into the blood capillaries and/or lymphatic vessels to become systemically bioavailable. Such can result from placement of the agent in the upper region of the dermis, i.e., the papillary dermis or in the upper portion of the relatively less vascular reticular dermis such that the agent readily diffuses into the papillary dermis. The controlled delivery of a biologically active agent in this dermal compartment below the papillary dermis in the reticular dermis, but sufficiently above the interface between the dermis and the subcutaneous tissue, should enable an efficient (outward) migration of the agent to the (undisturbed) vascular and lymphatic microcapillary bed (in the papillary dermis), where it can be absorbed into systemic circulation via these microcapillaries without being sequestered in transit by any other cutaneous tissue compartment. In some embodiments, placement of a biologically active agent predominately at a depth of at least about 0.3 mm, more preferably, at least about 0.4 mm and most preferably at least about 0.5 mm up to a depth of no more than about 2.5 mm, more preferably, no more than about 2.0 mm and most preferably no more than about 1.7 mm will result in rapid absorption of the agent. Although not intending to be bound by a particular mechanism of action, placement of the biologically active agent predominately at greater depths and/or into the lower portion of the reticular dermis may result in less effective uptake of the agent by the lymphatics as the agent will be slowly absorbed in the less vascular reticular dermis or in the subcutaneous region.

Biologically active agents, including diagnostic agents delivered in accordance with the methods of the invention will achieve higher maximum concentrations of the agents. The inventors have found that agents administered to the ID compartment are absorbed more rapidly, with bolus administration resulting in higher initial concentrations. Therefore, the dose can be reduced, providing an economic benefit, as well as a physiological benefit since lesser amounts of the drug or diagnostic agent has to be cleared by the body.

In accordance with the invention direct intradermal (ID) administration can be achieved using, for example, microneedle-based injection and infusion systems or any other means known to one skilled in the art to accurately target the intradermal compartment. Particular devices include those disclosed in WO 01/02178, published Jan. 10, 2002; and WO 02/02179, published Jan. 10, 2002, U.S. Pat. No. 6,494,865, issued Dec. 17, 2002 and U.S. Pat. No. 6,569,143 issued May 27, 2003 all of which are incorporated herein by reference in their entirety, as well as those exemplified in FIGS. 22-24. Micro-cannula- and microneedle-based methodology and devices are also described in U.S. application Ser. No. 09/606,909, filed Jun. 29, 2000, which is incorporated herein by reference in its entirety. Standard steel cannula can also be used for intra-dermal delivery using devices and methods as described in U.S. Ser. No. 417,671, filed Oct. 14, 1999, which is incorporated herein by reference in its entirety. These methods and devices include the delivery of agents through narrow gauge (30G or narrower) “micro-cannula” with a limited depth of penetration (typically ranging from 10 μm to 2 mm), as defined by the total length of the cannula or the total length of the cannula that is exposed beyond a depth-limiting hub feature.

The subject of intradermal delivery of the present invention is a mammal, preferably, a human. The biologically active agents delivered in accordance with the methods of the invention (with or without a tracer reagent) may be delivered into the intradermal compartment by a needle or cannula, usually from about 300 μm to about 5 mm long. Preferably, the needle or cannula is about 300 μm to about 1 mm long, with the outlet inserted into the skin of the subject to a depth of 1 mm to 3 mm. Preferably, a small gauge needle or cannula, between 30 and 36 gauge, preferably 31-34 gauge is used. The outlet of the needle or cannula is preferably inserted to a depth of 0.3 mm (300 um) to 1.5 mm.

Improved pharmacokinetic parameters using methods of the invention can be achieved using not only microdevice-based injection systems, but other delivery systems such as needle-less or needle-free ballistic injection of fluids or powders into the ID compartment, enhanced ionotophoresis through microdevices, and direct deposition of fluid, solids, or other dosing forms into the skin. In specific embodiments, the administration of the biologically active agent is accomplished through insertion of a needle or cannula into the intradermal compartment of the subject's skin.

The intradermal delivery of diagnostic agents in accordance with the present invention are particularly beneficial in the diagnosis of the diseases including chronic and acute diseases which include, but are not limited to, lymphoma, melanoma, leukemia, breast cancer, colorectal cancer, cancer metastasis, diseases of the lymphatic system, any disease affecting the lymph-nodes, e.g., axillary, politeal, lingual, viral diseases, e.g., HIV, immune disorders such as rejection, metabolic disorders, and infectious diseases. Although not intending to be bound by a particular mechanism of action, diagnostic agents delivered in accordance with the methods of the invention are taken up by the intradermal compartment and delivered to the lymphatic system where its recognition and binding indicate the presence of a cell or disease state. The present invention is useful for diagnostic procedures including, but not limited to, surgical methods, biopsies, non-invasive screening and image-guided biopsies.

The present invention provides improved methods for cancer detection and/or diagnosis by improving sensitivity, the amount of the agent deposited, tissue bioavailability, faster onset and clearance of the delivered diagnostic agent. Additionally methods of the invention are particularly improved over conventional cancer detection procedures for the detection of a tumor, e.g., breast tumor in a human subject, because more than 75% of the pre-selected volume of the diagnostic agent is deposited into the intradermal compartment, relative to when the same pre-selected volume is delivered to the intradermal compartment by the traditional methods of delivery of such agent, e.g., ID Mantoux method.

The present invention provides improved methods for current sentinel node biopsy procedure and mapping surgical procedure by improving the uptake and bioavailability of the diagnostic agents to the local lymphatic system. The invention provides a method for administration of at least one diagnostic agent for the detection of a tumor sentinal lymph node, e.g., breast tumor sentinal lymph node, or a lymph node that drains the tumor in a human subject, comprising delivering the agent into the intradermal compartment of the human subject's skin so that the agent is transported to the local lymphatic system. In other embodiments, the invention provides a method for administration of at least one diagnostic agent for the detection of a tumor sentinal lymph node or a lymph node that drains the tumor in a human subject, comprising delivering the agent into the intradermal compartment of the human subject's skin so that the agent has a higher tissue bioavailability compared to when the same agent is delivered by the ID Mantoux method. In yet other specific embodiments, the invention provides a method for administration of at least one diagnostic agent for the detection of a tumor sentinal lymph node, e.g., a breast tumor sentinal lymph node or a lymph node that drains the tumor in a human subject, comprising delivering the agent into the intradermal compartment of the human subject's skin so that the agent has a faster onset and clearance compared to when the same agent is delivered by the ID Mantoux method.

The methods of the instant invention provide improved prognostic methods using specific agents (versus non-specific agents) to assess therapeutic efficacy of a treatment regimen of a disease, for example by monitoring cellular genetic profiles in assessing gene regulation and expression over time. Traditionally in vitro analysis of cellular genetic profiles have been used to assess gene regulation and expression over time as a tool in assessing therapeutic efficacy. Such in vitro methods have numerous shortcomings including but not limited to inaccuracies, the removal of cells from the body can cause the destruction of RNA and DNA thereby altering the genetic profile in the specimen; information about the morphological locus of the genetic lesion is potentially lost using ex-vivo methods; and cell differentiation and regulation may be influenced by removal from the extracellular environment in vivo. By using the methods of the present invention, intradermal administration of specific diagnostic agents capable of associating and/or binding a specific marker for a disease provides for assessment of the disease as it exists in the patient. Thus, the methods taught by the present invention influence the choices of therapy available to the practitioner.

The methods of the invention are particularly useful in identification of impaired cellular metabolism of a disease or disorder, using for example genomics and proteomics technologies. In a specific embodiment, the methods of the invention provide improved methods for prognosis of cancer, particularly Diffuse Large B-cell lymphoma (DLBCL). Specific agents capable of distinguishing between DLBCL with and without active proliferative pathways can be delivered to the ID compartment and allowed to traffic throughout the lymphatic system. Binding of one agent would indicate a good or poor prognosis and thus enhanced effectiveness of therapy. In other specific embodiments, the methods of the invention provide improved methods for diagnosis of diseases associated with impaired signaling in the NFkb pathway by delivery of a specific agent to the ID compartment, allowing the agent to traffic to a particular cells, where it binds accordingly. The signal from the binding can be visualized in vivo. Binding of the agent indicates the presence of an impairment in the pathway and will allow assessment of the effectiveness of therapy and the onset of potential drug resistance as therapy progresses.

The methods of the invention are particularly useful for methods of integrated diagnosis and therapy preferably including require complementary and/or concurrent diagnostics and monitoring. Accurate diagnosis of a disease is largely an unmet need for example in oncology, where few diagnostic agents indicate which therapeutic choices will succeed with any reliability. The methods of the invention provide delivering agents which specifically recognize a cell, e.g., a cancer cell, in a particular tissue. Such agents include without limitation antibodies, preferably therapeutic monoclonal antibodies disclosed herein. In a specific embodiment, the invention encompasses delivering Herceptin, a monoclonal antibody specific for Her2/neu positive breast cancer to the ID compartment of a subject's skin for improved diagnosis and therapy. The methods of the invention provide improved diagnosis of cancer subjects over traditional methods of diagnostic of Her2/neu positive cancer cells, which identifies the population that will most benefit from this therapeutic treatment while eliminating others that would not. Currently, such in vitro diagnostic tests identifying the population that will most benefit from a particular therapeutic treatment produce “equivocal” or unclear results. By using the methods of the present invention, identification of the Her2/neu positive cells can be enhanced. Thus, in vivo intradermal administration of Herceptin or a nucleic acid that identifies the mRNA coding for Her2/neu, provides for the ability to identify those individuals suitable for integrated diagnostics and monitoring. Using the methods of this invention the cells are left intact providing a greater chance for positive identification.

The methods of the instant invention provide improved methods for tailoring therapies of a disease, disorder or infection using integrating diagnostic methods of the invention. The methods of the invention are applicable for current tailored and non-tailored treatment regimens. The methods of the invention allow a continuous monitoring of a treatment regimen in a subject. While tailored therapies of the future will require integrated diagnostics, current non-tailored treatment regimens could also benefit from tailored diagnostics of the instant invention. For example, those subjects diagnosed with large diffuse B cell lymphoma typically undergo CHOP therapy. Monitoring the effectiveness of this combined drug regimen is restricted to clinical changes and intermittent non-specific imaging and tissue biopsies. The ability to continually monitor treatment effectiveness would allow for earlier identification of drug resistance and metastasis. This could be accomplished with the administration of specific intradermal diagnostic reagents in the therapeutic cocktail or in combination with existing therapies.

The methods of the invention provide administration of formulations comprising one or more diagnostic agents in combination with one or more therapeutic agents. The present invention provides methods to target diagnostic agents and therapeutic agents to cells of interest. In a specific embodiment, the invention encompasses delivering a diagnostic agent combined with a therapeutic agent to the ID compartment of a subject's skin such that a specific action of the diagnostic agent triggers an action of the therapeutic agent. The combination of targeted diagnostic delivery with targeted therapeutics delivery in accordance with the methods of the invention provides for enhanced patient care. This embodiment teaches the advantages of combining intradermal therapeutic delivery with diagnostic agents. The combination of delivering a diagnostic and a therapeutic agent to the ID compartment, provides a powerful tool for improving the treatment of a disease in a subject.

In yet other embodiments, the invention enables the use of specific agents, e.g., diagnostic agents, for binding and/or detecting a cellular event or disease state in vivo. As a result, the invention provides screening methods to identify a specific agent needed to bind to the cell of interest. In some embodiments, the invention provides methods for in vivo screening of combinatorial libraries, both biological and chemical, to identify suitable agents (e.g., diagnostic target or moiety or therapeutic target or moiety) in the library for the purpose being tested. The ability to screen for agents in vivo using the methods of the instant invention enables identification of unique cellular and disease states.

In a specific embodiment, the invention provides using an animal model of interest, where libraries of agents can be injected intradermally and their effects monitored over time. Effects which can be monitored include for example relief of symptoms or binding to a tissue and/or cell of interest. In a preferred specific embodiment, an animal tumor model, e.g., a lymphoma mouse model could be used for screening biologically active agents, delivered intradermally that traffic to the lymph nodes. This would enable the detection of cancer cell states in vivo and possibly identify the active triggers for metastases and potential targets for therapeutic and diagnostic agents. These results would then be utilized to develop novel diagnostics for humans and other species.

5.1 Compositions of the Invention

The invention encompasses compositions comprising one or more biologically active agents in solution forms, particulate forms thereof and mixtures thereof. Compositions for use in the methods of the invention may be obtained from any species or generated by any recombinant DNA technology known to one skilled in the art. Compositions comprising one or more biologically active agents may be from different animal species including, limited but not to, swine, bovine, ovine, equine, etc. The chemical state of such agents may be modified by standard recombinant DNA technology to produce agents of different chemical formulas in different association states.

The biologically active agent used in the methods of the invention encompasses any molecule that either specifically or non-specifically binds a molecule in vivo and is capable of producing a biological effect in vivo. The biologically active agents may either be naturally occurring molecules or those derived using a synthetic process or recombinant process, using common methods known to one skilled in the art. Biologically active agents of the invention may recognize specifically or non-specifically a recognition moeity on a particular cell in a particular tissue. Often, these specific agents contain structural or functional properties in common with known biological entities. These biologically active agents may either be naturally occurring recognition molecules or those derived using a synthetic process or recombinant process, using common methods known to one skilled in the art.

In other embodiments, the biologically active agent is a biomimetic in nature, comprising naturally occurring structural motifs while incorporating additional or modified functional groups for transport, targeting, enhanced binding, stability, or detection.

Examples of biologically active agents that can be used in the methods of the instant invention include without limitation, immunoglobulins (e.g., Multi-specific Igs, Single chain Igs, Ig fragments), Proteins, Peptides (e.g., Peptide receptors, PNAs, Selectins, binding proteins (maltose binding protein, glucose binding protein)), Nucleotides, Nucleic Acids (e.g., PNAS, RNAs, modified RNA/DNA, aptamers), Receptors (e.g., Acetylcholine receptor), Enzymes (e.g., Glucose Oxidase, HIV Protease and reverse transcriptase), Carbohydrates (e.g, NCAMs, Sialic acids), Cells (e.g, Insulin & Glucose responsive cells), bacteriophags (e.g., filamentous phage), viruses (e.g., HIV), Chemospecific agents (e.g., Cyptands, Crown ethers, Boronates).

Particularly preferred biologically active agents that may be used in the instant invention are therapeutic antibodies that can be used diagnostically which include but are not limited to HERCEPTIN® (Trastuzumab) (Genentech, Calif.) which is a humanized anti-HER2 monoclonal antibody for the treatment of patients with metastatic breast cancer; REOPRO® (abciximab) (Centocor) which is an anti-glycoprotein IIb/IIIa receptor on the platelets for the prevention of clot formation; ZENAPAX® (daclizumab) (Roche Pharmaceuticals, Switzerland) which is an immunosuppressive, humanized anti-CD25 monoclonal antibody for the prevention of acute renal allograft rejection; PANOREX™ which is a murine anti-17-IA cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (ImClone System); VITAXIN™ which is a humanized anti-αVβ3 integrin antibody (Applied Molecular Evolution/MedImmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD20 IgG1 antibody (IDEC Pharm/Genentech, Roche/Zettyaku); LYMPHOCIDE™ which is a humanized anti-CD22 IgG antibody (Immunomedics); ICM3 is a humanized anti-ICAM3 antibody (ICOS Pharm); IDEC-114 is a primatied anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (C5) antibody (Alexion Pharm); D2E7 is a humanized anti-TNF-α antibody (CAT/BASF); CDP870 is a humanized anti-TNF-α Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CDP571 is a humanized anti-TNF-α IgG4 antibody (Celltech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); and CAT-152 is a human anti-TGF-β₂ antibody (Cambridge Ab Tech).

Other examples of antibodies that can be used in accordance with the instant invention are listed in Table 1 below.

TABLE 1
Monoclonal antibodies for Cancer Therapy that
can be used in accordance with the invention.

| Company | Product | Disease | Target |
|---|---|---|---|
| Abgenix | ABX-EGF | Cancer | EGF receptor |
| AltaRex | OvaRex | ovarian cancer | tumor antigen CA125 |
| | BravaRex | metastatic | tumor antigen MUC1 |
| | | cancers | |
| Antisoma | Theragyn | ovarian cancer | PEM antigen |
| | (pemtumomabytrrium- | | |
| | 90) | | |
| | Therex | breast cancer | PEM antigen |
| Boehringer | blvatuzumab | head & neck | CD44 |
| Ingelheim | | cancer | |
| Centocor/J&J | Panorex | Colorectal | 17-1A |
| | | cancer | |
| | ReoPro | PTCA | gp IIIb/IIIa |
| | ReoPro | Acute MI | gp IIIb/IIIa |
| | ReoPro | Ischemic stroke | gp IIIb/IIIa |
| Corixa | Bexocar | NHL | CD20 |
| CRC | MAb, idiotypic 105AD7 | colorectal cancer | gp72 |
| Technology | | vaccine | |
| Crucell | Anti-EpCAM | cancer | Ep-CAM |
| Cytoclonal | MAb, lung cancer | non-small cell | NA |
| | | lung cancer | |
| Genentech | Herceptin | metastatic breast | HER-2 |
| | | cancer | |
| | Herceptin | early stage | HER-2 |
| | | breast cancer | |
| | Rituxan | Relapsed/refractory | CD20 |
| | | low-grade or | |
| | | follicular NHL | |
| | Rituxan | intermediate & | CD20 |
| | | high-grade NHL | |
| | MAb-VEGF | NSCLC, | VEGF |
| | | metastatic | |
| | MAb-VEGF | Colorectal | VEGF |
| | | cancer, | |
| | | metastatic | |
| | AMD Fab | age-related | CD18 |
| | | macular | |
| | | degeneration | |
| | E-26 (2^{nd} gen. IgE) | allergic asthma | IgE |
| | | & rhinitis | |
| IDEC | Zevalin (Rituxan + yttrium- | low grade of | CD20 |
| | 90) | follicular, | |
| | | relapsed or | |
| | | refractory, | |
| | | CD20-positive, | |
| | | B-cell NHL and | |
| | | Rituximab- | |
| | | refractory NHL | |
| ImClone | Cetuximab + innotecan | refractory | EGF receptor |
| | | colorectal | |
| | | carcinoma | |
| | Cetuximab + cisplatin & | newly diagnosed | EGF receptor |
| | radiation | or recurrent head | |
| | | & neck cancer | |
| | Cetuximab + gemcitabine | newly diagnosed | EGF receptor |
| | | metastatic | |
| | | pancreatic | |
| | | carcinoma | |
| | Cetuximab + cisplatin + 5FU | recurrent or | EGF receptor |
| | or Taxol | metastatic head | |
| | | & neck cancer | |
| | Cetuximab + carboplatin + paclitaxel | newly diagnosed | EGF receptor |
| | | non-small cell | |
| | | lung carcinoma | |
| | Cetuximab + cisplatin | head & neck | EGF receptor |
| | | cancer | |
| | | (extensive | |
| | | incurable local- | |
| | | regional disease | |
| | | & distant | |
| | | metasteses) | |
| | Cetuximab + radiation | locally advanced | EGF receptor |
| | | head & neck | |
| | | carcinoma | |
| | BEC2 + Bacillus | small cell lung | mimics ganglioside |
| | Calmette Guerin | carcinoma | GD3 |
| | BEC2 + Bacillus | melanoma | mimics ganglioside |
| | Calmette Guerin | | GD3 |
| | IMC-1C11 | colorectal cancer | VEGF-receptor |
| | | with liver | |
| | | metasteses | |
| ImmonoGen | nuC242-DM1 | Colorectal, | nuC242 |
| | | gastric, and | |
| | | pancreatic | |
| | | cancer | |
| ImmunoMedics | LymphoCide | Non-Hodgkins | CD22 |
| | | lymphoma | |
| | LymphoCide Y-90 | Non-Hodgkins | CD22 |
| | | lymphoma | |
| | CEA-Cide | metastatic solid | CEA |
| | | tumors | |
| | CEA-Cide Y-90 | metastatic solid | CEA |
| | | tumors | |
| | CEA-Scan (Tc-99m- | colorectal cancer | CEA |
| | labeled arcitumomab) | (radioimaging) | |
| | CEA-Scan (Tc-99m- | Breast cancer | CEA |
| | labeled arcitumomab) | (radioimaging) | |
| | CEA-Scan (Tc-99m- | lung cancer | CEA |
| | labeled arcitumomab) | (radioimaging) | |
| | CEA-Scan (Tc-99m- | intraoperative | CEA |
| | labeled arcitumomab) | tumors (radio | |
| | | imaging) | |
| | LeukoScan (Tc-99m- | soft tissue | CEA |
| | labeled sulesomab) | infection | |
| | | (radioimaging) | |
| | LymphoScan (Tc-99m- | lymphomas | CD22 [symbol] |
| | labeled) | (radioimaging) | |
| | AFP-Scan (Tc-99m- | liver 7 gem-cell | AFP |
| | labeled) | cancers | |
| | | (radioimaging) | |
| Intracel | HumaRAD-HN (+yttrium- | head & neck | NA |
| | 90) | cancer | |
| | HumaSPECT | colorectal | NA |
| | | imaging | |
| Medarex | MDX-101 (CTLA-4) | Prostate and | CTLA-4 |
| | | other cancers | |
| | MDX-210 (her-2 | Prostate cancer | HER-2 |
| | overexpression) | | |
| | MDX-210/MAK | Cancer | HER-2 |
| MedImmune | Vitaxin | Cancer | αvβ₃ |
| Merck KGaA | MAb 425 | Various cancers | EGF receptor |
| | IS-IL-2 | Various cancers | Ep-CAM |
| Millennium | Campath (alemtuzumab) | chronic | CD52 |
| | | lymphocytic | |
| | | leukemia | |
| NeoRx | CD20-streptavidin (+biotin- | Non-Hodgkins | CD20 |
| | yttrium 90) | lymphoma | |
| | Avidicin (albumin + NRLU13) | metastatic | NA |
| | | cancer | |
| Peregrine | Oncolym (+iodine-131) | Non-Hodgkins | HLA-DR 10 beta |
| | | lymphoma | |
| | Cotara (+iodine-131) | unresectable | DNA-associated |
| | | malignant | proteins |
| | | glioma | |
| Pharmacia | C215 (+staphylococcal | pancreatic | NA |
| Corporation | enterotoxin) | cancer | |
| | MAb, lung/kidney cancer | lung & kidney | NA |
| | | cancer | |
| | nacolomab tafenatox | colon & | NA |
| | (C242 + staphylococcal | pancreatic | |
| | enterotoxin) | cancer | |
| Protein Design | Nuvion | T cell | CD3 |
| Labs | | malignancies | |
| | SMART M195 | AML | CD33 |
| | SMART 1D10 | NHL | HLA-DR antigen |
| Titan | CEAVac | colorectal | CEA |
| | | cancer, | |
| | | advanced | |
| | TriGem | metastatic | GD2-ganglioside |
| | | melanoma & | |
| | | small cell lung | |
| | | cancer | |
| | TriAb | metastatic breast | MUC-1 |
| | | cancer | |
| Trilex | CEAVac | colorectal | CEA |
| | | cancer, | |
| | | advanced | |
| | TriGem | metastatic | GD2-ganglioside |
| | | melanoma & | |
| | | small cell lung | |
| | | cancer | |
| | TriAb | metastatic breast | MUC-1 |
| | | cancer | |
| Viventia | NovoMAb-G2 | Non-Hodgkins | NA |
| Biotech | radiolabeled | lymphoma | |
| | Monopharm C | colorectal & | SK-1 antigen |
| | | pancreatic | |
| | | carcinoma | |
| | GlioMAb-H (+gelonin | gliorna, | NA |
| | toxin) | melanoma & | |
| | | neuroblastoma | |
| Xoma | Rituxan | Relapsed/refractory | CD20 |
| | | low-grade or | |
| | | follicular NHL | |
| | Rituxan | intermediate & | CD20 |
| | | high-grade NHL | |
| | ING-1 | adenomcarcinoma | Ep-CAM |

In one specific embodiment, the invention encompasses compositions comprising biologically active agents comprising one or more diagnostic agents. In another specific embodiment, the invention encompasses compositions comprising biologically active agents which comprise at least one diagnostic and at least one therapeutic agent. In one embodiment, the biologically active agent comprises a marker that identifies the cell type of a particular disease or disorder (e.g., a cancer), along with a therapeutic agent, e.g., an agent capable of killing diseased cells. For example, a marker identifying an undesirable cell type may be conjugated with a toxin capable of inactivating or killing the target cells.

Therapeutic agents that may be used in the compositions of the invention include but are not limited to chemotherapeutic agents, radiation therapeutic agents, hormonal therapeutic agents, immunotherapeutic agents, immunomodulatory agents, anti-inflammatory agents, antibiotics, anti-viral agents, and cytotoxic agents.

Non-limiting examples of anti-inflammatory agents include non-steroidal anti-inflammatory drugs (NSAIDs), steroidal anti-inflammatory drugs, beta-agonists, anticholingeric agents, and methyl xanthines. Examples of NSAIDs include, but are not limited to, aspirin, ibuprofen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™). Such NSAIDs function by inhibiting a cyclooxgenase enzyme (e.g., COX-1 and/or COX-2). Examples of steroidal anti-inflammatory drugs include, but are not limited to, glucocorticoids, dexamethasone (DECADRON™), cortisone, hydrocortisone, prednisone (DELTASONE™), prednisolone, triamcinolone, azulfidine, and eicosanoids such as prostaglandins, thromboxanes, and leukotrienes.

Examples of immunomodulatory agents include, but are not limited to, methothrexate, ENBREL, REMICADE™, leflunomide, cyclophosphamide, cyclosporine A, and macrolide antibiotics (e.g., FK506 (tacrolimus)), methylprednisolone (MP), corticosteroids, steriods, mycophenolate mofetil, rapamycin (sirolimus), mizoribine, deoxyspergualin, brequinar, malononitriloamindes (e.g., leflunamide), T cell receptor modulators, and cytokine receptor modulators, corticosteroids, cytokine agonists, cytokine antagonists, and cytokine inhibitors.

Examples of antibiotics include, but are not limited to, macrolide (e.g., tobramycin (Tobi®)), a cephalosporin (e.g., cephalexin (Keflex®), cephradine (Velosef®), cefuroxime (Ceftin®), cefprozil (Cefzil®), cefaclor (Ceclor®), cefixime (Suprax®) or cefadroxil (Duricef®)), a clarithromycin (e.g., clarithromycin (Biaxin®)), an erythromycin (e.g., erythromycin (EMycin®)), a penicillin (e.g., penicillin V (V-Cillin K® or Pen Vee K®)) or a quinolone (e.g., ofloxacin (Floxin®), ciprofloxacin (Cipro®) or norfloxacin (Noroxin®)),aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefinetazole, and cefminox), monobactams (e.g., aztreonam, carumonam, and tigemonam), oxacephems (e.g., flomoxef, and moxalactam), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o-benethamine, penicillin 0, penicillin V, penicillin V benzathine, penicillin V hydrabamine, penimepicycline, and phencihicillin potassium), lincosamides (e.g., clindamycin, and lincomycin), amphomycin, bacitracin, capreomycin, colistin, enduracidin, enviomycin, tetracyclines (e.g., apicycline, chlortetracycline, clomocycline, and demeclocycline), 2,4-diaminopyrimidines (e.g., brodimoprim), nitrofurans (e.g., furaltadone, and furazolium chloride), quinolones and analogs thereof (e.g., cinoxacin, clinafloxacin, flumequine, and grepagloxacin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), cycloserine, mupirocin, chloramphenicols, erythromycin, penicillin, streptomycin, vancomycin, trimethoprimsulfamethoxazols, and tuberin.

Examples of anti-viral agents include, but are not limited to, protease inhibitors, nucleoside reverse transcriptase inhibitors, non-nucleoside reverse transcriptase inhibitors and nucleoside analogs, zidovudine, acyclovir, gangcyclovir, vidarabine, idoxuridine, trifluridine, and ribavirin, as well as foscamet, amantadine, rimantadine, saquinavir, indinavir, amprenavir, lopinavir, ritonavir, the alpha-interferons; adefovir, clevadine, entecavir, and pleconaril.

Other therapeutic agents which can be used with the present invention include but are not limited to Alpha-1 anti-trypsin, Anti-Angiogenesis agents, Antisense, butorphanol, Calcitonin and analogs, Ceredase, COX-II inhibitors, dermatological agents, dihydroergotamine, Dopamine agonists and antagonists, Enkephalins and other opioid peptides, Epidermal growth factors, Erythropoietin and analogs, Follicle stimulating hormone, G-CSF, Glucagon, GM-CSF, granisetron, Growth hormone and analogs (including growth hormone releasing hormone), Growth hormone antagonists, Hirudin and Hirudin analogs such as Hirulog, IgE suppressors, Insulin, insulinotropin and analogs, Insulin-like growth factors, Interferons, Interleukins, Luteinizing hormone, Luteinizing hormone releasing hormone and analogs, Heparins, Low molecular weight heparins and other natural, modified, or synthetic glycoaminoglycans, M-CSF, metoclopramide, Midazolam, Monoclonal antibodies, Pegylated antibodies, Pegylated proteins or any proteins modified with hydrophilic or hydrophobic polymers or additional functional groups, Fusion proteins, Single chain antibody fragments or the same with any combination of attached proteins, macromolecules, or additional functional groups thereof, Narcotic analgesics, nicotine, Non-steroid anti-inflammatory agents, Oligosaccharides, ondansetron, Parathyroid hormone and analogs, Parathyroid hormone antagonists, Prostaglandin antagonists, Prostaglandins, Recombinant soluble receptors, scopolamine, Serotonin agonists and antagonists, Sildenafil, Terbutaline, Thrombolytics, Tissue plasminogen activators, TNF, and TNF antagonist, the vaccines, with or without carriers/adjuvants, including prophylactics and therapeutic antigens (including but not limited to subunit protein, peptide and polysaccharide, polysaccharide conjugates, toxoids, genetic based vaccines, live attenuated, reassortant, inactivated, whole cells, viral and bacterial vectors) in connection with, addiction, arthritis, cholera, cocaine addiction, diphtheria, tetanus, HIB, Lyme disease, meningococcus, measles, mumps, rubella, varicella, yellow fever, Respiratory syncytial virus, tick borne japanese encephalitis, pneumococcus, streptococcus, typhoid, influenza, hepatitis, including hepatitis A, B, C and E, otitis media, rabies, polio, HIV, parainfluenza, rotavirus, Epstein Barr Virus, CMV, chlamydia, non-typeable haemophilus, moraxella catarrhalis, human papilloma virus, tuberculosis including BCG, gonorrhoea, asthma, atheroschlerosis malaria, E-coli, Alzheimer's Disease, H. Pylori, salmonella, diabetes, cancer, herpes simplex, human papilloma and the like other substances including all of the major. therapeutics such as agents for the common cold, Anti-addiction, anti-allergy, anti-emetics, anti-obesity, antiosteoporeteic, anti-infectives, analgesics, anesthetics, anorexics, antiarthritics, antiasthmatic agents, anticonvulsants, antidepressants, antidiabetic agents, antihistamines, anti-inflammatory agents, antimigraine preparations, antimotion sickness preparations, antinauseants, antineoplastics, antiparkinsonism drugs, antipruritics, antipsychotics, antipyretics, anticholinergics, benzodiazepine antagonists, vasodilators, including general, coronary, peripheral and cerebral, bone stimulating agents, central nervous system stimulants, hormones, hypnotics, immunosuppressives, muscle relaxants, parasympatholytics, parasympathomimetrics, prostaglandins, proteins, peptides, polypeptides and other macromolecules, psychostimulants, sedatives, and sexual hypofunction and tranquilizers.

A biologically active agent, e.g., a diagnostic agent, may be conjugated to a therapeutic moiety such as a cytotoxin (e.g., a cytostatic or cytocidal agent), a therapeutic agent or a radioactive element (e.g., alpha-emitters, gamma-emitters, etc.). Cytotoxins or cytotoxic agents include any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine), prednisone and adriomycin.

Moreover, a biologically active agent can be conjugated to therapeutic moieties such as a radioactive materials or macrocyclic chelators useful for conjugating radiometal ions (see above for examples of radioactive materials). In certain embodiments, the macrocyclic chelator is 1,4,7,10-tetraazacyclododecane-N,N′,N″,N″-tetraacetic acid (DOTA) which can be attached to the antibody via a linker molecule. Such linker molecules are commonly known in the art and described in Denardo et al., 1998, Clin Cancer Res. 4:2483-90; Peterson et al., 1999, Bioconjug. Chem. 10:553; and Zimmerman et al., 1999, Nucl. Med. Biol. 26:943-50 each incorporated by reference in their entireties.

Techniques for conjugating such therapeutic moieties to biologically active agents, e.g., antibodies, are well known; see, e.g., Arnon et al., “Monoclonal Antibodies For Immunotargeting Of Drugs In Cancer Therapy”, in Monoclonal Antibodies And Cancer Therapy, Reisfeld et al. (eds.), 1985, pp. 243-56, Alan R. Liss, Inc.); Hellstrom et al., “Antibodies For Drug Delivery”, in Controlled Drug Delivery (2nd Ed.), Robinson et al. (eds.), 1987, pp. 623-53, Marcel Dekker, Inc.); Thorpe, “Antibody Carriers Of Cytotoxic Agents In Cancer Therapy: A Review”, in Monoclonal Antibodies '84: Biological And Clinical Applications, Pinchera et al. (eds.), 1985, pp. 475-506); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), 1985, pp. 303-16, Academic Press; and Thorpe et al., Immunol. Rev., 62:119-58, 1982.

In some embodiments, the compositions of the invention comprise an effective amount of a biologically active agent and one or more other additives. Additives that may be used in the compositions of the invention include for example, wetting-agents, emulsifying agents, or pH buffering agents. The compositions of the invention may contain one or more other excipients such as saccharides and polyols. Additional examples of pharmaceutically acceptable carriers, diluents, and other excipients are provided in Remington's Pharmaceutical Sciences (Mack Pub. Co. N.J. current edition, all of which is incorporated herein by reference in its entirety.

The invention encompasses compositions in which the biologically active agent is in a particulate form, i.e., is not fully dissolved in solution. In some embodiments, at least 30%, at least 50%, at least 75% of the biologically active agent is in particulate form. Although not intending to be bound by a particular mode of action, compositions of the invention in which a biologically active agent is in particulate form have at least one agent which facilitates the precipitation of the agent. Precipitating agents that may be employed in the compositions of the invention may be proteinacious, e.g., protamine, a cationic polymer, or non-proteinacious, e.g., zinc or other metals or polymers.

In some embodiments, a tracer agent may be concurrently administered with the biologically active agent to facilitate the tracing and examination of the biologically active agent. The tracer agent may include, but is not limited to, visible dyes, fluorescent dyes, radioisotopes, microbubbles, or magnetic spin labels. Such tracer agents can be easily observed by the conventional techniques. Detection of the labeled agents or the tracer agents may be accomplished using ex vivo or in vivo, invasive or non-invasive, using methods known in the art.

The form of the biologically active agent to be delivered or administered include solutions thereof in pharmaceutically acceptable diluents or solvents, emulsions, suspensions, gels, particulates such as micro- and nanoparticles either suspended or dispersed, as well as in-situ forming vehicles of the same. The compositions of the invention may be in any form suitable for intradermal delivery. In one embodiment, the intradermal composition of the invention is in the form of a flowable, injectible medium, i.e., a low viscosity composition that may be injected in a syringe or pen. The flowable injectible medium may be a liquid. Alternatively the flowable injectible medium is a liquid in which particulate material is suspended, such that the medium retains its fluidity to be injectible and syringable, e.g., can be administered in a syringe.

The invention encompasses formulations comprising at least one biologically active agent wherein the the concentration of the agent is between about 20 ug/mL to 100 mg/mL. In a specific embodiment, the concentration of the agent is is about 10 mg/mL. In another specific embodiment, the concentration of the agent is about 100 mg/mL. In some embodiments, the amount of the at agent delivered in accordance with the methods of the invention is between about 5 and 10 ug.

The invention also includes compositions comprising particle reagents for diagnostic and/or therapeutic use and methods of delivery thereof. In brief, particles of defined shape and surface characteristics may be suspended in liquid media and delivered for example through micro needles to the intradermal compartment, e.g., generally less than 5 mm below the epidermis and preferably between 1 and 3 mm below the epidermis. These particles are then transported through the lymphatic vasculature to lymph nodes. Particle migration rate may be contingent on size and surface charge.

As used herein, the term “particles” includes any formed element comprising monomers, polymers, lipids, amphiphiles, fatty acids, steroids, proteins, and other materials known to aggregate, self-assemble or which can be processed into particles. Particles also include unilamelar, multilamelar, random tortuous path and solid morphologies including but not limited to liposomes, microcrystalline materials, particulate MRI contrast agents, polymeric beads (i.e., latex and HEMA), but most preferably hollow particles, such as microbubbles, which are particularly useful for ultrasonic imaging.

In one preferred embodiment, the invention encompasses particles comprising of one or more biologically active agents including therapeutic and diagnostic agents which may result in site selective non-invasive dissolution of said particles to deliver the agent. In a specific embodiment, the invention encompasses compositions comprising an ultrasound contrast agent (e.g., a microbubble) comprising a therapeutic and/or diagnostic agent, e.g., doxorubicin. Although not intending to be bound by a particular mechanism of action, once introduced the particles are actively or passively trafficked into the area and regional draining lymph nodes. As the particles move into these tissues an ultrasound probe detects their presence and, at the appropriate frequency, breaks the particle open; its contents then diffuse into nearby tissues allowing for high local agent concentration only at the disease locus without need for systemic delivery. Additionally, the particle may further comprise a diagnostic agent so that dispersion of the agent is limited to the immediate tissue for additional analysis.

The advantages of such particle delivery systems include but are not limited to, (1) improved targeting of the lymphatic system tissue via targeted ID delivery. Using such delivery systems, disease response occurs in the lymphatic tissue and direct access to this process may offer greater effectiveness of therapeutics and improved diagnostic capabilities; (2) Improved therapeutic and diagnostic outcomes. Local delivery of therapeutics to tissue of greatest interest offers the possibility of improved clinical outcomes due to altered PK and PD profiles. With local delivery in accordance with the methods of the invention less agent than traditional systemic delivery may be used in order to achieve the desired clinical or diagnostic outcome with the associated decrease in side effects. The methods of the invention result in an increased sensitivity and speed for diagnostic assessment due to local delivery of high concentration agent.

Particles as described herein are delivered intradermally and may be, non-specific non-tissue binding, or specific tissue and/or cell binding (that is, the particle may bind to a particular biological entity or may have a targeting molecule attached to it), and may be associated with therapeutic or diagnostic moieties via various methods. The particles themselves may be the therapeutic or diagnostic agent or they may encapsulate, entrap, or bind the therapeutic or diagnostic agent. The invention encompasses all drug classes and diagnostic agents. The therapeutic or diagnostic agents used in the methods and compositions of the invention may or may not be cell or tissue targeted.

In some specific embodiments, the particles comprise one or more diagnostic agents. Although not intending to be bound by a particular particles provide signal amplification needed for diagnosis of rare events using imaging methods known in the art and disclosed herein.

In some embodiments, particle reagents may further comprise therapeutic agents which are carried with the particles into the lymphatic system and delivered at rates determined by particle composition. In some specific embodiments, the particles comprise therapeutic agents in combination with one or more diagnostic agents. Although not intending to be bound by a particular mechanism of action, particles provide for extended targeted release of agents to particular tissues and/or organs rather than release to the general circulation. Consequently, toxicity is reduced and therapeutic effect is maximized.

In particularly preferred embodiments, the compositions used in the methods of the invention comprise of nanoparticles.

One preferred embodiment of this aspect of the invention relates to a composition comprising small non-specific microbubbles and a method for delivering the composition using intradermal methods to a particular tissue, e.g., lymphatic tissue, or a particular organ. Although not intending to be bound by a particular mechanism of action microbubbles are rapidly transported through the lymphatic circulation and may be detected using for example ultrasonic imaging. The invention thus provides improved methods for detecting cancer metastases for example to sentinel lymph nodes, and/or improved methods for evaluating lymphedema, e.g., a common morbidity associated with extensive axillary lymph node dissection. The methods of the invention are improved over conventional cancer diagnostics such as those disclosed in, e.g., Creager, A. J.; Geisinger, K. R.; Shiver, S. A.; Perier, N. D.; Shen, P.; Shaw, J.; Young, P. R.; Levine, E. A. “Intraoperative Evaluation of Sentinel Lymph Nodes for Metastatic Breast Carcinoma by Imprint Cytology” Mod Pathol 2002, 15(11), 1140-1146.

In other specific embodiments the invention encompasses hypoxia detection via intradermal delivery of oxygen responsive particles.

The intradermal compositions of the present invention can be prepared as unit dosage forms. A unit dosage per vial may contain 0.1 to 0.5 mL of the composition. In some embodiments, a unit dosage form of the intradermal compositions of the invention may contain 50 μL to 100 μL, 50 μL to 200 μL, or 50 μL to 500 μL of the composition. If necessary, these preparations can be adjusted to a desired concentration by adding a sterile diluent to each vial. Compositions administered in accordance with the methods of the invention are not administered in volumes whereby the intradermal space might become overloaded leading to partitioning to one or more other compartments, such as the SC compartment.

5.2 Diagnostic Uses

The present invention provides improved methods for diagnosis and/or detection of a disease, disorder, or infection by improving sensitivity, the amount of the agent deposited, tissue bioavailability, faster onset and clearance of the delivered biologically active agent, e.g., diagnostic agent. The biologically active agents, particularly diagnostic agents disclosed herein can be used for diagnostic purposes to detect, diagnose, or monitor diseases, disorders or infections. The invention provides a method for administration of at least one diagnostic agent for the detection of a disease, particularly cancer, comprising delivering the agent into the ID compartment of a subject's skin at a controlled rate, volume and pressure so that the agent is deposited into the ID compartment and taken up by the lymphatic vasculature.

The methods of the invention encompass administering a diagnostically effective, preferably non-toxic amount of an agent to a mammal, such that the agent is imageable and detectable with sufficient resolution through the methods disclosed herein and known to one skilled in the art, e.g., ultrasound or magnetic resonance imaging to permit visualization of intranodal architecture. Preferably the agents administered in accordance with the methods of the invention are deposited in a particular tissue, e.g., in the lymph nodes; and the agent is imaged in the subject. The agent may be images within about 2 weeks of said administration, within about 1 months of said administration, within about 2 months of said administration, or within about 3 months of said administration.

In some embodiments, the invention provides a method for the detection or diagnosis of a disease, disorder or infection, comprising: (a) delivering one or more diagnostic agents to the ID compartment of the subject's skin; (b) assaying the expression of a specific gene known to have aberrant expression or levels resulting in the disease, disorder, or infection in a subject using one or more agents that specifically bind to a cell expressing the specific gene; and (b) comparing the level of the expression of the gene with a control level, e.g., levels in normal tissue samples, whereby an increase in the assayed level compared to the control level is indicative of the disease, disorder or infection.

One aspect of the invention is the detection and diagnosis of a disease, disorder, or infection in a human. In one embodiment, diagnosis comprises: (a) administering to a subject an effective amount of a labeled biologically active agent by delivering the agent to the ID compartment of the subject's skin so that the agent specifically binds a cell that resides in the target tissue; (b) waiting for a time interval following the administration of the agent for permitting the labeled agent to preferentially concentrate at sites in the subject where specific binding to the target tissue occurs (and for unbound labeled agent to be cleared to background level); (c) determining background level; and (d) detecting the labeled agent in the subject, such that detection of labeled agent above the background level indicates that the subject has the disease, disorder, or infection. In accordance with this embodiment, the agent is labeled with an imaging moiety which is detectable using an imaging system known to one of skill in the art. Background level can be determined by various methods including, comparing the amount of labeled agent detected to a standard value previously determined for a particular system.

The present invention provides improved methods for current sentinel node biopsy procedure and mapping surgical procedure by improving the uptake and the bioavaialability of the diagnostic agents to the local lymphatic system. The invention provides a method for administration of at least one diagnostic agent for the detection of a tumor, e.g., breast tumor, or a lymph node that drains the tumor in a human subject, comprising delivering the agent into the intradermal compartment of the human subject's skin so that the agent is transported to the local lymphatic system. In other embodiments, the invention provides a method for administration of at least one diagnostic agent for the detection of a tumor, or a lymph node that drains the tumor in a human subject, comprising delivering the agent into the intradermal compartment of the human subject's skin so that the agent has a higher tissue bioavailability compared to when the same agent is delivered by the ID Mantoux method. In yet other specific embodiments, the invention provides a method for administration of at least one diagnostic agent for the detection of a tumor, e.g., a breast tumor, or a lymph node that drains the tumor in a human subject, comprising delivering the agent into the intradermal compartment of the human subject's skin so that the agent has a faster onset and clearance compared to when the same agent is delivered by the ID Mantoux method.

The methods of the invention are particularly improved over conventional cancer detection procedures for the detection of a tumor, e.g., breast tumor or a lymph node that drains the tumor in a human subject, because more than 75% of the pre-selected volume of the diagnostic agent is deposited into the intradermal compartment, relative to when the same pre-selected volume is delivered to the intradermal compartment by the traditional methods of delivery of such agents, e.g., ID Mantoux method.

In a specific embodiment, the invention encompasses a diagnostic method for cancer comprising the following: antibody specific for a particular cell type, i.e., breast cancer, labeled with a dye that is detectable upon exposure to a specific light source is injected intradermally into and around the tissue of interest. The surgeon using a unique light source (hand held or incorporated into another instrument (e.g., specially designed eyeglasses)) follows the path of the labeled antibody in the lymph nodes looking for metastases and cancer spread. In alternative embodiments, the label is radioactive or magnetic with an appropriate external source to track the label, and in some cases, may be one that is not capable of being detected until the specific agent binds to its target.). The diagnostic agents of the invention are particularly useful for cancer prognosis since oxygen concentration proximal to tumors often indicates susceptibility to radiation (see, e.g., Lo et al., 1995, Biochemistry 20, 11,727-11730) and photodynamic therapies (see, e.g., Mcllroy et al., 1998, J Photochem Photobiol, 43, 47-55).

In one embodiment, the present invention provides a method particularly useful for diagnosis of cancer metastasis. In the diagnostic method, a biologically active agent is intradermally delivered to a location suspected of having a tumor, and the biologically active agent is transported to the local lymphatic system so that the lymphatic system, including the lymph nodes draining the location, are identified. Microexamination is then performed on the identified lymph nodes to determine whether cancer cells have migrated into the lymph nodes, i.e., that metastasis has occurred. Further transport beyond the lymphatic tissue provides a unique mode for rapid delivery of biologically active agents and enhanced tissue-bioavailability. For example, ProstaScint™ (Cytogen) is an ¹¹¹In labeled monoclonal antibody used for staging prostate cancer; ⁹⁹TC labeled anti CD-15 monoclonal antibodies have been used for highly sensitive and specific identification of equivocal appendicitis (Kipper, S. L. et. al. Journal of Nuclear Medicine 2000 41(3), 449-455). The invention encompasses administration of Cytogen to the ID compartment of a subject's skin to provide an improved diagnostic application of prostate cancer.

The invention encompasses a method for administration of at least one diagnostic agent for the detection of a tumor or a lymph node that drains the tumor to a human subject, comprising delivering the agent into the intradermal compartment of the human subject's skin so that the agent is transported to the local lymphatic system. Preferably the diagnostic agents delivered in accordance with the methods of the invention have a higher tissue bioavailability, faster onset and clearance compared to when the same agent is delivered by the ID Mantoux method. Most preferably the amount of the pre-selected dose of the agent deposited in the lymphatic tissue is increased by at least 100%, at least 150%, at least 200%, at least 250%, at least 300%, at least 350% compared to when the same agent is delivered by the ID Mantoux method. In yet other preferred embodiments, the amount of the pre-selected dose of the agent deposited in the lymphatic tissue is increased by at least 100%, at least 150%, at least 200%, at least 250%, at least 300%, at least 350% compared to when the same agent is delivered to a deeper tissue compartment, e.g., SC compartment, IM compartment.

In one preferred embodiment, the invention provides an improved method for the diagnosis of metastasis of tumor cells, comprising: delivering a biologically active agent that is transported in vivo to the lymphatic system, tracing the biologically active agent to determine the lymphatic system draining the location, and microexamining the lymphatic system for metastasis.

It is an object of the invention to provide a method for delivering a biologically active agent, e.g., a diagnostic agent, to a subject comprising administering a biologically active agent into an intradermal compartment of the subject's skin, wherein the biologically active agent specifically associates with or binds to a marker of a disease or disorder. Preferably, the biologically active agent demonstrates improved biological kinetics or biological dynamics or tissue-bioavailability compared to conventional methods of delivery.

The present invention provides a method for diagnosing a disease, disorder, or infection having a specific marker, by administering a biologically active agent for said disease or disorder using the methods disclosed herein, tracing the biologically active agent and determining whether any specific binding of said agent occurs, such binding indicating the probability of said disease or disorder. The biologically active agents of the invention can be used diagnostically to, monitor the development or progression of a disease, disorder or infection as part of a clinical testing procedure to, e.g., determine the efficacy of a given treatment regimen.

The methods of the instant invention provide improved prognostic methods using specific agents (versus non-specific agents) to assess therapeutic efficacy of a treatment regimen of a disease, for example, by monitoring cellular genetic profiles in assessing gene regulation and expression over time. Traditionally, in vitro analysis of cellular genetic profiles have been used to assess gene regulation and expression over time as a tool in assessing therapeutic efficacy. Such in vitro methods have numerous shortcomings including, but not limited to, inaccuracies, the removal of cells from the body can cause the destruction of RNA and DNA thereby altering the genetic profile in the specimen, information about the morphological locus of the genetic lesion is potentially lost using ex-vivo methods, and cell differentiation and regulation may be influenced by removal from the extracellular environment in vivo. By using the methods of the present invention, intradermal administration of specific diagnostic agents capable of associating and/or binding a specific marker for a disease provides for assessment of disease as it exists in the patient. Thus, the methods taught by the present invention influence the choices of therapy available to the practitioner.

The methods of the invention are particularly useful for methods of integrated diagnosis and therapy. Accurate diagnosis of a disease is largely an unmet need for example in oncology, where few diagnostic agents indicate which therapeutic choices will succeed with any reliability. The methods of the invention provide improved methods for integrated diagnosis and therapy by administration of formulations comprising one or more diagnostic agents in combination with one or more therapeutic agents. The present invention provides methods to target diagnostic agents and therapeutic agents to a particular cell in a particular tissue. In a specific embodiment, the invention encompasses delivering formulations comprising one or more diagnostic agents in combination with one or more therapeutic agents to the ID compartment of a subject's skin such that a specific action of the diagnostic agent triggers an action, e.g., biological effect, of the therapeutic agent. The combination of targeted diagnostic delivery with targeted therapeutics delivery in accordance with the methods of the invention provides for enhanced patient care. This embodiment teaches the advantages of combining intradermal therapeutic delivery with diagnostic agents. The combination of delivering a diagnostic and a therapeutic agent to the ID compartment provides a powerful tool for improving the treatment of a disease in a subject.

In some embodiments, the invention encompasses repeated administration of one or more labeled specific agents (e.g., an antibody) intradermally in the area of interest, prior to external screening process (i.e., mammography or other imaging system). Each specific agent is then monitored during the procedure. Specific agents may be a part of a diagnostic kit with pre-filled syringe(s) or delivery device(s). In one embodiment, monitoring of a disease, disorder or infection is carried out by repeating the method for diagnosing the disease, disorder or infection, for example, one month after initial diagnosis, six months after initial diagnosis, or one year after initial diagnosis.

The present invention also provides a method for delivering biologically active agent to a subject, in which the biologically active agent is administered to the intradermal compartment of the subject and is transported in vivo to the local lymphatic system. Thus, the biologically active agent reaches the local lymphatic system before it is excreted, degraded, or metabolized by, for example, the liver, kidneys, or spleen. In some embodiments, the biologically active agent comprises an immunoglobulin, a protein or peptide, a nucleotide, polynucleotide or nucleic acid, a ligand for a neuron receptor, an enzyme, a carbohydrate, cellular therapeutic agent, a chemospecific agent, or a combination thereof. Further, a tracer agent may be concurrently administered with the biologically active agent, or the biologically active agent itself may be labeled so that it can be traced in vivo. The tracing and examination of the tracer agent or self-labeled biologically active agent may be conducted by ex vivo flow cytometry, histological methods, or other ex vivo techniques known in the art, or in vivo using, SPECT, PET, MRI, fluorescence, luminescence, bioluminescence, optical imaging, photoacoustic imaging, RAMAN and SERS or other in vivo imaging techniques known in the art.

For agents that are administered by injection, the limits of the targeted tissue depth are controlled inter alia by the depth to which the needle or cannula outlet is inserted, the exposed height (vertical rise) of the outlet, the volume administered, and the rate of administration. Suitable parameters can be determined by persons of skill in the art without undue experimentation.

The invention encompasses administering one or more diagnostic agents employing surgical and non-surgical methods. For suitable agents, imaging via an external monitor (i.e., MRI, PET, CAT Scan, or mammography) outside of the surgical site is used. Non-surgical methods may be use for diseases which include, but are not limited to, breast cancer, lymphoma, colorectal and prostate cancer imaging and screening, early detection of rare cells indicative of a disease state, chronic diseases such as rheumatoid arthritis, and blood borne pathogens such as HIV.

Detection can be facilitated by coupling the biologically active agent to a detectable substance. Examples of detectable substances include various enzymes, prosthetic groups, fluorescent materials, luminescent materials, bioluminescent materials, radioactive materials, positron emitting metals, visisble dyes, fluorescent dyes, radioisotopes, magnetic spin labels, and non-radioactive paramagnetic metal ions. The detectable substance may be coupled or conjugated either directly to the biologically active agent or indirectly, through an intermediate (such as, for example, a linker known in the art) using techniques known in the art. See, for example, U.S. Pat. No. 4,741,900 for metal ions which can be conjugated to antibodies for use as diagnostics according to the present invention. Such diagnosis and detection can be accomplished by coupling the biologically active agent to detectable substances including, but not limited to, various enzymes, enzymes including, but not limited to, horseradish peroxidase, alkaline phosphatase, beta-galactosidase, or acetylcholinesterase; prosthetic group complexes such as, but not limited to, streptavidin/biotin and avidin/biotin; fluorescent materials such as, but not limited to, umbelliferone, fluorescein, fluorescein isothiocyanate, rhodamine, dichlorotriazinylamine fluorescein, dansyl chloride or phycoerythrin; luminescent material such as, but not limited to, luminol; bioluminescent materials such as, but not limited to, luciferase, luciferin, and aequorin; radioactive material such as, but not limited to, bismuth (²¹³Bi), carbon (¹⁴C), chromium (⁵¹Cr), cobalt (⁵⁷Co), fluorine (¹⁸F), gadolinium (¹⁵³Gd, ¹⁵⁹Gd), gallium (⁶⁸Ga, ⁶⁷Ga), germanium (⁶⁸Ge), holmium (¹⁶⁶Ho), indium (¹¹⁵In, ¹¹³In, ¹¹²In, ¹¹¹In), iodine (¹³¹I, ¹²⁵I, ¹²³I, ¹²¹I), lanthanium (¹⁴⁰La), lutetium (¹⁷⁷Lu), manganese (⁵⁴Mn), molybdenum (⁹⁹Mo), palladium (¹⁰³Pd), phosphorous (³²P), praseodymium (¹⁴²Pr), promethium (¹⁴⁹Pm), rhenium (¹⁸⁶Re, ¹⁸⁸Re), rhodium (¹⁰⁵Rh), ruthemium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), thallium (²⁰¹Ti), tin (¹¹³Sn, ¹¹⁷Sn), tritium (³H), xenon (¹³³Xe), ytterbium (¹⁶⁹Yb, ¹⁷⁵Yb), yttrium (⁹⁰Y), zinc (⁶⁵Zn); positron emitting metals using various positron emission tomographies, and non-radioactive paramagnetic metal ions.

The invention encompasses any detection method known in the art and exemplified herein including but not limited to ex vivo or in vivo, invasive or non-invasive. Detection of the labeled agents and biologically active agents in accordance with the methods of the invention may be done using optical methods (e.g., time resolved and life time fluorescence spectroscopy, luminescence, or bioluminescence, chemiluminescence); flow cytometry, fluorescence in the infrared region, histological examination, ultrasonography, photoacoustics spectroscopy, Raman spectroscopy, and surface enhanced raman spectroscopy. In preferred embodiments, the examination and tracing of the location of the biologically active agent is by way of in vivo imaging. Any suitable method of in vivo imaging known in the art, including, for example, SPECT, optical imaging, photoacoustic imaging, RAMAN and SERS CAT, PET, may be used in the methods of the invention.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ^{99m}Tc. The labeled antibody will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982); which is incorporated herein by reference in its entirety.) Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 5 to 10 days.

Presence of the labeled molecule can be detected in the subject using methods known in the art for in vivo scanning. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), single photon emission computer tomography (SPECT), X-Ray, Optical (spectrophotometric) imaging and sonography.

In a specific embodiment, the biologically active agent is labeled with a radioisotope and is detected in the patient using a radiation responsive surgical instrument (Thurston et al., U.S. Pat. No. 5,441,050). In another embodiment, the biologically active agent is labeled with a fluorescent compound and is detected in the patient using a fluorescence responsive scanning instrument. In another embodiment, the biologically active agent is labeled with a positron emitting metal and is detected in the patient using positron emission-tomography. In yet another embodiment, the biologically active agent is labeled with a paramagnetic label and is detected in a patient using magnetic resonance imaging (MRI).

The invention encompasses in vivo imaging agents delivered in accordance to the methods of the invention. Such agents can be detected using the appropriate imaging modality. Imaging modalities include but are not limited to ultrasound, MRI, CT, PET, SPECT, Fluorescent, Chemiluminescent, Bioluminescence, X-Ray, and Photoacoustic imaging. The invention encompasses in vivo imaging of a disease, disorder, or infection using the biologically active agents and other agents disclosed herein, e.g., tracer agents, imaging agents. Once a biologically active agent is delivered to a subject, the subject may be imaged appropriately which can be during the injection, immediately after injection, and/or at an appointed times post injection. The images obtained can be continuous (real time) or episodic in manner. The images can be used to locate structures, i.e., lymph nodes, identify architectural features including obstructions, flow rate of the agent, and identify rare events.

The present invention encompasses delivering contrast agents suitable for imaging by one or more imaging techniques. Any contrast agent known in the art is contemplated within the methods and compositions of the invention. In some embodiments, the contrast agents are in particulate form and are adapted to be preferentially taken up by the lymphatic system upon administration. These contrast agents can be radiopaque materials, MRI imaging agents, ultrasound imaging agents, and any other contrast agent suitable for a device that images an animal body. Contrast agents for use in the methods of the invention are preferably nontoxic and/or non-radioactive. There are two major classes of contrast agents: paramagnetic and superparamagnetic; each of which is contemplated within the methods of the invention. Paramagnetic agents have unpaired electron spins that facilitate relaxation of nuclei, usually water protons, that can closely approach them (within 1 nm). These agents decrease both T1 and T2, are effective in uM concentrations, and can be incorporated in chelates with favorable biodistribution and toxicity profiles. Schering's patented product, GdDTPA (gadolinium diethylenetriaminepentaacetic acid), is an outstanding example of several commercially available such agents. In some embodiments the contrast agents are incorporated into macromolecules to avoid uptake by the systemic circulation. Combination with albumin, other biological molecules of appropriate size, latex, dextran, polystyrene or other nontoxic natural or synthetic polymer, or encapsulation in liposomes, can be accomplished using methods known to those skilled in the art.

The invention further encompasses non-specific contrast agents including but not limited to: MRI contrast agents (e.g., gadolinium, paramagnetic particles, super-paramagnetic particles), ultrasound contrast agents (e.g., microbubbles), CT contrast agents (e.g., radiolabels), X-Ray contrast agents (e.g., Iodine), PET contrast agents (e.g., any 2 photon emitter, F19, Fluoro-deoxy-glucose), Photoacoustic contrast agents (e.g., dyes, various light absorbing molecules), Optical contrast agents (e.g., Fluorescent: CY5, squaraines, near infrared dyes, i.e. indocyanine green, lanthanide fluors (e.g., Europium, Turbium).

In a particular example, microbubble ultrasound contrast agent is delivered as described herein. An ultrasound probe is positioned either at the injection site or at a regional lymph node site. Although not intending to be bound by a particular mechanism of action the contrast agent is delivered to the intradermal compartments and immediately travels through the lymphatic vessels and to the lymph node. The ultrasound probe detects the contrast agent as it passes beneath the probe. Both diagnostic flow rate and architecture information, including obstructions, can be obtained. In this embodiment, the images can be obtained continuously (real time) or in an episodic manner.

In specific embodiments, the invention encompasses a method for diagnosing a disease affecting the lymph nodes which is improved over traditional lymphography methods known in the art. The methods of the invention encompasses using ultrasound or magnetic resonance imaging. The methods of the invention encompass administering a diagnostically effective, non-toxic amount, non-radioactive contrast agent to a mammal, such that the agent is imageable with sufficient resolution through ultrasound or magnetic resonance imaging to permit visualization of intranodal architecture; permitting the contrast agent to localize in the lymph nodes; and imaging the lymph nodes of the mammal in which said contrast agent has localized with magnetic resonance imaging or ultrasound within about 2 weeks of said administration, within about 1 months of said administration, within about 2 months of said administration, or within about 3 months of said administration.

In some embodiments, magnetic resonance images further comprise an additional step of making sure to pre-image the subject prior to injection of the agent, e.g., contrast agent. In some embodiments, Multiple images post injection are obtained over time and compared to the pre-image. The invention encompasses methods for detection and location of lymph nodes, as well as information concerning other tissues, organs and biological entities using methods disclosed herein and known to those skilled in the art, e.g., CT, PET, SPECT, Optical (e.g., Fluorescent, Chemiluminescent) and X-Ray imaging.

5.2.1 Diseases

The methods of the invention can be used for improved diagnosis of cancers and related disorders including but not limited to, the following: Leukemias including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors including but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including but not limited to, pheochromocytom and adrenocortical carcinoma; thyroid cancer such as but not limited to papillary or follicular thyroid cancer, medullary thyroid cancer and anaplastic thyroid cancer; pancreatic cancer, including but not limited to, insulinoma, gastrinoma, glucagonoma, vipoma, somatostatin-secreting tumor, and carcinoid or islet cell tumor; pituitary cancers including but not limited to, Cushing's disease, prolactin-secreting tumor, acromegaly, and diabetes insipius; eye cancers including but not limited to, ocular melanoma such as iris melanoma, choroidal melanoma, and cilliary body melanoma, and retinoblastoma; vaginal cancers, including but not limited to, squamous cell carcinoma, adenocarcinoma, and melanoma; vulvar cancer, including but not limited to, squamous cell carcinoma, melanoma, adenocarcinoma, basal cell carcinoma, sarcoma, and Paget's disease; cervical cancers including but not limited to, squamous cell carcinoma, and adenocarcinoma; uterine cancers including but not limited to, endometrial carcinoma and uterine sarcoma; ovarian cancers including but not limited to, ovarian epithelial carcinoma, borderline tumor, germ cell tumor, and stromal tumor; esophageal cancers including but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers including but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers including but not limited to hepatocellular carcinoma and hepatoblastoma, gallbladder cancers including but not limited to, adenocarcinoma; cholangiocarcinomas including but not limited to, pappillary, nodular, and diffuse; lung cancers including but not limited to, non-small cell lung cancer, squamous cell carcinoma (epidermoid carcinoma), adenocarcinoma, large-cell carcinoma and small-cell lung cancer; testicular cancers including but not limited to, germinal tumor, seminoma, anaplastic, classic (typical), spermatocytic, nonseminoma, embryonal carcinoma, teratoma carcinoma, choriocarcinoma (yolk-sac tumor), prostate cancers including but not limited to, adenocarcinoma, leiomyosarcoma, and rhabdomyosarcoma; penal cancers; oral cancers including but not limited to, squamous cell carcinoma; basal cancers; salivary gland cancers including but not limited to, adenocarcinoma, mucoepidermoid carcinoma, and adenoidcystic carcinoma; pharynx cancers including but not limited to, squamous cell cancer, and verrucous; skin cancers including but not limited to, basal cell carcinoma, squamous cell carcinoma and melanoma, superficial spreading melanoma, nodular melanoma, lentigo malignant melanoma, acral lentiginous melanoma; kidney cancers including but not limited to, renal cell cancer, adenocarcinoma, hypemephroma, fibrosarcoma, transitional cell cancer (renal pelvis and/or uterer); Wilms' tumor; bladder cancers including but not limited to, transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America). Accordingly, the methods and agents of the invention are also useful in the diagnosis of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Berketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosafcoma, rhabdomyoscarama, and osteosarcoma; and other tumors, including melanoma, xenoderma pegmentosum, keratoactanthoma, seminoma, thyroid follicular cancer and teratocarcinoma. It is also contemplated that cancers caused by aberrations in apoptosis would also be treated by the methods and compositions of the invention. Such cancers may include but not be limited to follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, diagnosed more effectively by the methods and compositions of the invention in the ovary, bladder, breast, colon, lung, skin, pancreas, or uterus. In other specific embodiments, sarcoma, melanoma, or leukemia is diagnosed more effectively by the methods and compositions of the invention.

Cancers associated with the cancer antigens may diagnosed more effectively by administration of the agents of the invention, For example, but not by way of limitation, cancers associated with the following cancer antigen may be diagnosed more effectively by the methods and compositions of the invention. KS ¼ pan-carcinoma antigen (Perez and Walker, 1990, J. Immunol. 142:32-37; Bumal, 1988, Hybridoma 7(4):407-415), ovarian carcinoma antigen (CA125) (Yu et al., 1991, Cancer Res. 51(2):48-475), prostatic acid phosphate (Tailor et al., 1990, Nucl. Acids Res. 18(1):4928), prostate specific antigen (Henttu and Vihko, 1989, Biochem. Biophys. Res. Comm. 10(2):903-910; Israeli et al., 1993, Cancer Res. 53:227-230), melanoma-associated antigen p97 (Estin et al., 1989, J. Natl. Cancer Instit. 81(6):445-44), melanoma antigen gp75 (Vijayasardahl et al., 1990, J. Exp. Med. 171(4):1375-1380), high molecular weight melanoma antigen (HMW-MAA) (Natali et al., 1987, Cancer 59:55-3; Mittelman et al., 1990, J. Clin. Invest. 86:2136-2144)), prostate specific membrane antigen, carcinoembryonic antigen (CEA) (Foon et al., 1994, Proc. Am. Soc. Clin. Oncol. 13:294), polymorphic epithelial mucin antigen, human milk fat globule antigen, Colorectal tumor-associated antigens such as: CEA, TAG-72 (Yokata et al., 1992, Cancer Res. 52:3402-3408), CO17-1A (Ragnhammar et al., 1993, Int. J. Cancer 53:751-758); GICA 19-9 (Herlyn et al., 1982, J. Clin. Immunol. 2:135), CTA-1 and LEA, Burkitt's lymphoma antigen-38.13, CD19 (Ghetie et al., 1994, Blood 83:1329-1336), human B-lymphoma antigen-CD20 (Reff et al., 1994, Blood 83:435-445), CD33 (Sgouros et al., 1993, J. Nucl. Med. 34:422-430), melanoma specific antigens such as ganglioside GD2 (Saleh et al., 1993, J. Immunol., 151, 3390-3398), ganglioside GD3 (Shitara et al., 1993, Cancer Immunol. Immunother. 36:373-380), ganglioside GM2 (Livingston et al., 1994, J. Clin. Oncol. 12:1036-1044), ganglioside GM3 (Hoon et al., 1993, Cancer Res. 53.5244-5250), tumor-specific transplantation type of cell-surface antigen (TSTA) such as virally-induced tumor antigens including T-antigen DNA tumor viruses and envelope antigens of RNA tumor viruses, oncofetal antigen-alpha-fetoprotein such as CEA of colon, bladder tumor oncofetal antigen (Hellstrom et al., 1985, Cancer. Res. 45:2210-2188), differentiation antigen such as human lung carcinoma antigen L6, L20 (Hellstrom et al., 1986, Cancer Res. 46:3917-3923), antigens of fibrosarcoma, human leukemia T cell antigen-Gp37 (Bhattacharya-Chatterjee et al., 1988, J. of Immun. 141:1398-1403), neoglycoprotein, sphingolipids, breast cancer antigen such as EGFR (Epidermal growth factor receptor), HER2 antigen (p185^{HER2}), polymorphic epithelial mucin (PEM) (Hilkens et al., 1992, Trends in Bio. Chem. Sci. 17:359), malignant human lymphocyte antigen-APO-1 (Bernhard et al., 1989, Science 245:301-304), differentiation antigen (Feizi, 1985, Nature 314:53-57) such as I antigen found in fetal erthrocytes and primary endoderm, I (Ma) found in gastric adencarcinomas, M18 and M39 found in breast epithelium, SSEA-1 found in myeloid cells, VEP8, VEP9, Myl, VIM-D5, and D₁56-22 found in colorectal cancer, TRA-1-85 (blood group H), C14 found in colonic adenocarcinoma, F3 found in lung adenocarcinoma, AH6 found in gastric cancer, Y hapten, Le^{y} found in embryonal carcinoma cells, TL5 (blood group A), EGF receptor found in A431 cells, E₁ series (blood group B) found in pancreatic cancer, FC10.2 found in embryonal carcinoma cells, gastric adenocarcinoma, CO-514 (blood group Le^{a}) found in adenocarcinoma, NS-10 found in adenocarcinomas, CO-43 (blood group Le^{b}), G49, EGF receptor, (blood group ALe^{b}/Le^{y}) found in colonic adenocarcinoma, 19.9 found in colon cancer, gastric cancer mucins, T₅A₇ found in myeloid cells, R₂₄ found in melanoma, 4.2, G_{D3}, D1.1, OFA-1, G_{M2}, OFA-2, G_{D2}, M1:22:25:8 found in embryonal carcinoma cells and SSEA-3, SSEA-4 found in 4-8-cell stage embryos. In another embodiment, the antigen is a T cell receptor derived peptide from a cutaneous T cell lymphoma (see Edelson, 1998, The Cancer Journal 4:62).

The biologically active agents, particularly diagnostic agents disclosed herein can be used for diagnostic purposes to detect, diagnose, or monitor infections (e.g., lymphangitis, pneumonia, slymphadenitis, streptococcus, RSV). Infectious diseases that can be detected, diagnosed, or monitored by the agents of the invention are caused by infectious agents including but not limited to viruses, bacteria, fungi, protozae, and viruses.

Viral diseases that can be detected, diagnosed, or monitored using the agents of the invention in conjunction with the methods of the present invention include, but are not limited to, those caused by hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, huntavirus, coxsackie virus, mumps virus, measles virus, rubella virus, polio virus, small pox, Epstein Barr virus, human immunodeficiency virus type I (HIV-I), human immunodeficiency virus type II (HIV-II), and agents of viral diseases such as viral miningitis, encephalitis, dengue or small pox.

Bacterial diseases that can be detected, diagnosed, or monitored using the agents of the invention in conjunction with the methods of the present invention, that are caused by bacteria include, but are not limited to, mycobacteria rickettsia, mycoplasma, neisseria, S. pneumonia, Borrelia burgdorferi (Lyme disease), Bacillus antracis (anthrax), tetanus, streptococcus, staphylococcus, mycobacterium, tetanus, pertissus, cholera, plague, diptheria, chlamydia, S. aureus and legionella.

Protozoal diseases that can be detected, diagnosed, or monitored using the agents of the invention in conjunction with the methods of the present invention, that are caused by protozoa include, but are not limited to, leishmania, kokzidioa, trypanosoma or malaria.

Parasitic diseases that can be detected, diagnosed, or monitored using the agents of the invention in conjunction with the methods of the present invention, that are caused by parasites include, but are not limited to, chlamydia and rickettsia.

5.3 Intradermal Administration of Biologically Active Agents

The invention encompasses methods for intradermal delivery of biologically active agents, particularly diagnostic agents, described and exemplified herein to the intradermal compartment of a subject's skin, preferably by directly and selectively targeting the intradermal compartment, particularly the dermal vasculature, without entirely passing through it. Once the biologically active agents, particularly diagnostic agents for use in the methods of the invention are prepared, the agent is typically transferred to an injection device for intradermal delivery, e.g., a syringe or pen. The biologically active agents, particularly diagnostic agents may be in a commercial preparation, such as a vial or cartridge, specifically designed for intradermal injection. The biologically active agents, particularly diagnostic agents of the invention are administered using any of the intradermal devices and methods known in the art or disclosed in WO 01/02178, published Jan. 10, 2002; and WO 02/02179, published Jan. 10, 2002, U.S. Pat. No. 6,494,865, issued Dec. 17, 2002 and U.S. Pat. No. 6,569,143 issued May 27, 2003 all of which are incorporated herein by reference in their entirety.

The actual method by which the intradermal administration of the biologically active agents, particularly diagnostic agents is targeted to the intradermal compartment is not critical as long as it penetrates the skin of a subject to the desired targeted depth within the intradermal compartment without passing through it. In most cases, the device will penetrate the skin to a depth of about 0.5-2 mm. The invention encompasses conventional injection needles, catheters or microneedles of all known types, employed singularly or in multiple needle arrays. The dermal access means may comprise needle-less devices including ballistic injection devices. The terms “needle” and “needles” as used herein are intended to encompass all such needle-like structures with any bevel or even without a point. The term microneedles as used herein are intended to encompass structure 30 gauge and smaller, typically about 31-50 gauge when such structures are cylindrical in nature. Non-cylindrical structures encompass by the term microneedles would therefore be of comparable diameter and include pyramidal, rectangular, octagonal, wedged, and other geometrical shapes. They too may have any bevel, combination of bevels or may lack a point. The methods of the invention also include ballistic fluid injection devices, powder-jet delivery devices, piezoelectric, electromotive, electromagnetic assisted delivery devices, gas-assisted delivery devices, of which directly penetrate the skin to provide access for delivery or directly deliver agents to the targeted location within the dermal compartment.

Preferably however, the device has structural means for controlling skin penetration to the desired depth within the intradermal compartment. This is most typically accomplished by means of a widened area or hub associated with the shaft of the dermal-access means that may take the form of a backing structure or platform to which the needles are attached. The length of microneedles as dermal-access means are easily varied during the fabrication process and are routinely produced in less than 2 mm length. Microneedles are also a very sharp and of a very small gauge, to further reduce pain and other sensation during the injection or infusion. They may be used in the invention as individual single-lumen microneedles or multiple microneedles may be assembled or fabricated in linear arrays or two-dimensional arrays as to increase the rate of delivery or the amount of agent delivered in a given period of time. The needle may eject its agent from the end, the side or both. Microneedles may be incorporated into a variety of devices such as holders and housings that may also serve to limit the depth of penetration. The dermal-access means of the invention may also incorporate reservoirs to contain the agent prior to delivery or pumps or other means for delivering the drug or other agent under pressure. Alternatively, the device housing the dermal-access means may be linked externally to such additional components.

The intradermal methods of administration comprise microneedle-based injection and infusion systems or any other means to accurately target the intradermal compartment. The intradermal methods of administration encompass not only microdevice-based injection means, but other delivery methods such as needle-less or needle-free ballistic injection of fluids or powders into the intradermal compartment, enhanced ionotophoresis through microdevices, and direct deposition of fluid, solids, or other dosing forms into the skin.

The invention provides a method for an improved method of delivering biologically active agents, particularly diagnostic agents into the intradermal compartment of a subject's skin comprising the steps of providing a delivery device, e.g., such as those exemplified in FIGS. 22-24, including a needle cannula having a forward needle tip and the needle cannula being in fluid communication with an agent contained in the delivery device and including a limiter portion surrounding the needle cannula and the limiter portion including a skin engaging surface, with the needle tip of the needle cannula extending from the limiter portion beyond the skin engaging surface a distance equal to approximately 0.5 mm to approximately 3.0 mm and the needle cannula having a fixed angle of orientation relative to a plane of the skin engaging surface of the limiter portion, inserting the needle tip into the skin of an animal and engaging the surface of the skin with the skin engaging surface of the limiter portion, such that the skin engaging surface of the limiter portion limits penetration of the needle cannula tip into the dermis layer of the skin of the animal, and expelling the agent from the delivery device through the needle cannula tip into the skin of the animal.

In other preferred embodiments, the invention encompass selecting an injection site on the skin of the subject, cleaning the injection site on the skin of the subject prior to expelling the biologically active agents, particularly diagnostic agents from the delivery device into the skin of the subject. In addition, the method comprises filling the delivery device with the biologically active agents, particularly diagnostic agents of the invention. Further, the method comprises pressing the skin engaging surface of the limiter portion against the skin of the subject and applying pressure, thereby stretching the skin of the subject, and withdrawing the needle cannula from the skin after injecting the agent. Still further, the step of inserting the forward tip into the skin is further defined by inserting the forward tip into the skin to a depth of from approximately 1.0 mm to approximately 2.0 mm, and most preferably into the skin to a depth of 1.5 mm+0.2 to 0.3 mm. FIGS. 25-28 exemplify specific embodiments of the intradermal methods of the invention.

In a preferred embodiment, the step of inserting the forward tip into the skin of the animal is further defined by inserting the forward tip into the skin at an angle being generally perpendicular to the skin within about fifteen degrees, with the angle most preferably being generally ninety degrees to the skin, within about five degrees, and the fixed angle of orientation relative to the skin engaging surface is further defined as being generally perpendicular. In the preferred embodiment, the limiter surrounds the needle cannula, having a generally planar flat skin engaging surface. Also, the delivery device comprises a syringe having a barrel and a plunger received within the barrel and the plunger being depressable to expel the agent from the delivery device through the forward tip of the needle cannula, e.g., see FIGS. 22-24.

In a preferred embodiment, expelling the biologically active agents, particularly diagnostic agents, from the delivery device is further defined by grasping the hypodermic needle with a first hand and depressing the plunger with an index finger of a second hand and expelling the agent from the delivery device by grasping the hypodermic needle with a first hand and depressing the plunger on the hypodermic needle with a thumb of a second hand, with the step of inserting the forward tip into the skin of the animal further defined by pressing the skin of the animal with the limiter. In addition, the method may further comprise the step of attaching a needle assembly to a tip of the barrel of the syringe with the needle assembly including the needle cannula and the limiter, and may comprise the step of exposing the tip of the barrel before attaching the needle assembly thereto by removing a cap from the tip of the barrel. Alternatively, the step of inserting the forward tip of the needle into the skin of the subject may be further defined by simultaneously grasping the hypodermic needle with a first hand and pressing the limiter against the skin of the animal thereby stretching the skin of the animal, and expelling the agent by depressing the plunger with an index finger of the first hand or expelling the agent by depressing the plunger with a thumb of the first hand. The method further encompasses withdrawing the forward tip of the needle cannula from the skin of the subject after the agent has been injected into the skin of the subject. Still further, the method encompasses inserting the forward tip into the skin preferably to a depth of from approximately 1.0 mm to approximately 2.0 mm, and most preferably to a depth of 1.5 mm+0.2 to 0.3 mm.

Preferably, prior to inserting the needle cannula 24 (see FIGS. 22-24), an injection site upon the skin of the subject is selected and cleaned. Subsequent to selecting and cleaning the site, the forward end 40 of the needle cannula 24 is inserted into the skin of the subject at an angle of generally 90 degrees until the skin engaging surface 42 contacts the skin. The skin engaging surface 42 prevents the needle cannula 42 from passing through the dermis layer of the skin and injecting the agent into the subcutaneous layer. While the needle cannula 42 is inserted into the skin, the agent is intradermally injected. The agent may be prefilled into the syringe 60, either substantially before and stored therein just prior to making the injection. Several variations of the method of performing the injection may be utilized depending upon individual preferences and syringe type. In any event, the penetration of the needle cannula 42 is most preferably no more than about 1.5 mm because the skin engaging surface 42 prevents any further penetration.

Also, during the administration of an intradermal injection, the forward end 40 of the needle cannula 42 is embedded in the dermis layer of the skin which results in a reasonable amount of back pressure during the injection of the biologically active agents, particularly diagnostic agents of the invention. In order to reach this pressure with a minimal amount of force having to be applied by the user to the plunger rod 66 of the syringe, a syringe barrel 60 with a small inside diameter is preferred such as 0.183″ (4.65 mm) or less. The method of this invention thus comprises selecting a syringe for injection having an inside diameter of sufficient width to generate a force sufficient to overcome the back pressure of the dermis layer when the biologically active agents, particularly diagnostic agents is expelled from the syringe to make the injection.

In addition, since intradermal injections are typically carried out with small volumes of the biologically active agents, particularly diagnostic agents to be injected, i.e., on the order of no more than 0.5 ml, and preferably around 0.1 ml, a syringe barrel 60 with a small inside diameter is preferred to minimize dead compartment which could result in wasted agent captured-between the stopper 70 and the shoulder of the syringe after the injection is completed. Also, because of the small volumes of agents, e.g., on the order of 0.1 ml, a syringe barrel with a small inside diameter is preferred to minimize air head compartment between the level of the agent and the stopper 70 during process of inserting the stopper. Further, the small inside diameter enhances the ability to inspect and visualize the volume of the agents within the barrel of the syringe.

As shown in FIG. 25, the syringe 60 may be grasped with a first hand 112 and the plunger 66 depressed with the forefinger 114 of a second hand 116. Alternatively, the plunger 66 may be depressed by the thumb 118 of the second hand 116 while the syringe 60 is held by the first hand. In each of these variations, the skin of the subject is depressed, and stretched by the skin engaging surface 42 on the limiter 26. The skin is contacted by neither the first hand 112 nor the second hand 116.

An additional variation has proven effective for administering the intradermal injection of the present invention. This variation includes gripping the syringe 60 with the same hand that is used to depress the plunger 66. The syringe 60 being gripped with the first hand 112 while the plunger is simultaneously depressed with the thumb 120 of the first hand 112. This variation includes stretching the skin with the second hand 114 while the injection is being made. Alternatively, as shown in FIG. 26, the grip is reversed and the plunger is depressed by the forefinger 122 of the first hand 112 while the skin is being stretched by the second hand 116.

The methods of the invention result in improved pharmacokinetics of the administered agents. By “improved pharmacokinetics” it is meant that an enhancement of pharmacokinetic profile is achieved as measured, for example, by standard pharmacokinetic parameters such as time to maximal plasma concentration (Tₘₐₓ), the magnitude of maximal plasma concentration (Cₘₐₓ) or the time to elicit a minimally detectable blood or plasma concentration (T_{tag}). By enhanced absorption profile, it is meant that absorption is improved or greater as measured by such pharmacokinetic parameters. The measurement of pharmacokinetic parameters and determination of minimally effective concentrations are routinely performed in the art. Values obtained are deemed to be enhanced by comparison with a standard route of administration such as, for example, subcutaneous administration or intramuscular administration. In such comparisons, it is preferable, although not necessarily essential, that administration into the intradermal layer and administration into the reference site-such as subcutaneous administration involve the same dose levels, i.e., the same amount and concentration of agent as well as the same carrier vehicle and the same rate of administration in terms of amount and volume per unit time. Thus, for example, administration of a given agent into the dermis at a concentration such as 100 μg/mL and rate of 100 μL per minute over a period of 5 minutes would, preferably, be compared to administration of the same agent into the subcutaneous compartment at the same concentration of 100 μg/mL and rate of 100 μL per minute over a period of 5 minutes.

The above-mentioned PK and PD benefits are best realized by accurate direct targeting of the dermal capillary beds. This is accomplished, for example, by using microneedle systems of less than about 250 micron outer diameter, and less than 2 mm exposed length. Such systems can be constructed using known methods of various materials including steel, silicon, ceramic, and other metals, plastic, polymers, sugars, biological and or biodegradable materials, and/or combinations thereof.

It has been found that certain features of the intradermal administration methods provide clinically useful PK/PD and dose accuracy. For example, it has been found that placement of the needle outlet within the skin significantly affects PK/PD parameters. The outlet of a conventional or standard gauge needle with a bevel has a relatively large exposed height (the vertical rise of the outlet). Although the needle tip may be placed at the desired depth within the intradermal compartment, the large exposed height of the needle outlet causes the delivered agent to be deposited at a much shallower depth nearer to the skin surface. As a result, the agent tends to effuse out of the skin due to backpressure exerted by the skin itself and to pressure built up from accumulating fluid from the injection or infusion and to leak into the lower pressure regions of the skin, such as the subcutaneous tissue. That is, at a greater depth a needle outlet with a greater exposed height will still seal efficiently where as an outlet with the same exposed height will not seal efficiently when placed in a shallower depth within the intradermal compartment. Typically, the exposed height of the needle outlet will be from 0 to about 1 mm. A needle outlet with an exposed height of 0 mm has no bevel and is at the tip of the needle. In this case, the depth of the outlet is the same as the depth of penetration of the needle. A needle outlet that is either formed by a bevel or by an opening through the side of the needle has a measurable exposed height. It is understood that a single needle may have more than one opening or outlets suitable for delivery of agents to the dermal compartment.

It has also been found that by controlling the pressure of injection or infusion the high backpressure exerted during ID administration can be overcome. By placing a constant pressure directly on the liquid interface a more constant delivery rate can be achieved, which may optimize absorption and obtain the improved pharmacokinetics. Delivery rate and volume can also be controlled to prevent the formation of wheals at the site of delivery and to prevent backpressure from pushing the dermal-access means out of the skin and/or into the subcutaneous region. The appropriate delivery rates and volumes to obtain these effects may be determined experimentally using only ordinary skill. Increased spacing between multiple needles allows broader fluid distribution and increased rates of delivery or larger fluid volumes.

The administration methods useful for carrying out the invention include both bolus and infusion delivery of the biologically active agents to humans or animals subjects. A bolus dose is a single dose delivered in a single volume unit over a relatively brief period of time, typically less than about 10 minutes. Infusion administration comprises administering a fluid at a selected rate that may be constant or variable, over a relatively more extended time period, typically greater than about 10 minutes. To deliver an agent, the dermal-access means is placed adjacent to the skin of a subject providing directly targeted access within the intradermal compartment and the agent or agents are delivered or administered into the intradermal compartment where they can act locally or be absorbed by the bloodstream and be distributed systematically. The dermal-access means may be connected to a reservoir containing the agent or agents to be delivered.

Delivery from the reservoir into the intradermal compartment may occur either passively, without application of the external pressure or other driving means to the agent or agents to be delivered, and/or actively, with the application of pressure or other driving means. Examples of preferred pressure generating means include pumps, syringes, pens, elastomer membranes, gas pressure, piezoelectric, electromotive, electromagnetic or osmotic pumping, or Belleville springs or washers or combinations thereof. If desired, the rate of delivery of the agent may be variably controlled by the pressure-generating means.

In some embodiments, the invention encompasses methods for controlling the pharmacokinetics of administered biologically active agents by combining the advantages of delivery to two or more compartments or depths within skin. In particular, the invention provides a method for delivering a biologically active agent, particularly a diagnostic agent as described herein to the shallow SC and ID compartments to achieve a hybrid pK profile that has a portion similar to that achieved by ID delivery and another portion similar to that achieved by SC delivery. This provides rapid and high peak onset levels of the biologically active agent, particularly a diagnostic agent as well as a lower prolonged circulating level of the agent. Such methods are disclosed in U.S. application Ser. No. 10/429,973, filed May 6, 2003 which is incorporated herein by reference in its entirety. In some embodiments, the biologically active agent, particularly a diagnostic agent is delivered to a site or, sites that include two or more compartments. In other embodiments, biologically active agent, particularly a diagnostic agent is delivered to multiple sites that each include one or more compartments.

The methods of the invention encompass controlled delivery of the biologically active agent, particularly a diagnostic agent using algorithms having logic components that include physiologic models, rules based models or moving average methods, therapy pharmacokinetic models, monitoring signal processing algorithms, predictive control models, or combinations thereof.

The methods of the invention encompass a method for combinations of shallow SC and ID delivery to achieve improved PK outcomes. These outcomes are not achievable using solely one delivery compartment or another. Multiple site deposition via proper device configuration and/or dosing method may obtain unique and beneficial results. The underlying technical principle is that the PK outcome of microneedle delivery is specific to the deposition depth and patterning of the administered fluid, that such deposition can be controlled mechanically via device design and engineering or by technique such as fluid overloading of the ID compartment.

In addition, the invention includes needles (micro or otherwise) for SC injection having a length less than 5 mm length. Shallow SC delivery to a depth of about 3 mm yields almost identical PK to deep SC using traditional techniques. The utility of shallow SC delivery alone to yield more controlled profiles has never been exploited. In fact, previously depths of less than 5 mm have been considered to not be within the SC compartment.

Mixed delivery either by device design or technique results in biphasic or mixed kinetic profiling. Minor differences in device length (1 mm vs. 2 mm vs. 3 mm) 30 yield dramatic differences in PK outcomes. SC-like profiles can be obtained with needle lengths often assumed to locate the end of the needle within the ID compartment. Shallow SC delivery is more consistent and uniform in PK outcomes than standard SC delivery. The limits of the targeted tissue depth are controlled inter alia by the depth to which the needle or cannula outlet is inserted, the exposed height (vertical rise) of the outlet, the volume administered, and the rate of administration. Suitable parameters can be determined by persons of skill in the art without undue experimentation.

The invention encompasses administering the compositions of the invention intradermally as disclosed herein in combination with other routes of delivery including for example, subcutaneous-intradermal interface, intransal (IN), parenteral administration (e.g., intramuscular, intraperitoneal, intravenous and subcutaneous), epidural, and mucosal (e.g., intranasal and oral routes). The compositions may be administered by any convenient route, for example, by infusion or bolus injection, by absorption through epithelial or mucocutaneous linings (e.g., oral mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968; 5,985, 320; 5,985,309; 5,934,272; 5,874,064; 5,855,913; 5,290,540; and 4,880,078; and PCT Publication Nos. WO 92/19244; WO 97/32572; WO 97/44013; WO 98/31346; and WO 99/66903, each of which is incorporated herein by reference in its entirety.

5.3.1 Devices for Intradermal Administration

The biologically active agents, including the diagnostic agents of the invention are administered using any of the devices and methods known in the art or disclosed in WO 01/02178, published Jan. 10, 2002; and WO 02/02179, published Jan. 10, 2002, U.S. Pat. No. 6,494,865, issued Dec. 17, 2002 and U.S. Pat. No. 6,569,143 issued May 27, 2003 all of which are incorporated herein by reference in their entirety.

Preferably the devices for intradermal administration in accordance with the methods of the invention have structural means for controlling skin penetration to the desired depth within the intradermal space. This is most typically accomplished by means of a widened area or hub associated with the shaft of the dermal-access means that may take the form of a backing structure or platform to which the needles are attached. The length of microneedles as dermal-access means are easily varied during the fabrication process and are routinely produced in less than 2 mm length. Microneedles are also a very sharp and of a very small gauge, to further reduce pain and other sensation during the injection or infusion. They may be used in the invention as individual single-lumen microneedles or multiple microneedles may be assembled or fabricated in linear arrays or two-dimensional arrays as to increase the rate of delivery or the amount of substance delivered in a given period of time. The needle may eject its substance from the end, the side or both. Microneedles may be incorporated into a variety of devices such as holders and housings that may also serve to limit the depth of penetration. The dermal-access means of the invention may also incorporate reservoirs to contain the substance prior to delivery or pumps or other means for delivering the drug or other substance under pressure. Alternatively, the device housing the dermal-access means may be linked externally to such additional components.

The intradermal methods of administration comprise microneedle-based injection and infusion systems or any other means to accurately target the intradermal space. The intradermal methods of administration encompass not only microdevice-based injection means, but other delivery methods such as needle-less or needle-free ballistic injection of fluids or powders into the intradermal space, enhanced ionotophoresis through microdevices, and direct deposition of fluid, solids, or other dosing forms into the skin.

In some embodiments, the present invention provides to a delivery device including a needle assembly for use in making intradermal injections. The needle assembly has an adapter that is attachable to prefillable containers such as syringes and the like. The needle assembly is supported by the adapter and has a hollow body with a forward end extending away from the adapter. A limiter surrounds the needle and extends away from the adapter toward the forward end of the needle. The limiter has a skin engaging surface that is adapted to be received against the skin of an animal such as a human. The needle forward end extends away from the skin engaging surface a selected distance such that the limiter limits the amount or depth that the needle is able to penetrate through the skin of an animal

In a specific embodiment, the hypodermic needle assembly for use in the methods of the invention comprises the elements necessary to perform the present invention directed to an improved method delivering biologically active agents, including the diagnostic agents into the skin of a subject's skin, preferably a human subject's skin, comprising the steps of providing a delivery device including a needle cannula having a forward needle tip and the needle cannula being in fluid communication with an agent contained in the delivery device and including a limiter portion surrounding the needle cannula and the limiter portion including a skin engaging surface, with the needle tip of the needle cannula extending from the limiter portion beyond the skin engaging surface a distance equal to approximately 0.5 mm to approximately 3.0 mm and the needle cannula having a fixed angle of orientation relative to a plane of the skin engaging surface of the limiter portion, inserting the needle tip into the skin of an animal and engaging the surface of the skin with the skin engaging surface of the limiter portion, such that the skin engaging surface of the limiter portion limits penetration of the needle cannula tip into the dermis layer of the skin of the animal, and expelling the substance from the drug delivery device through the needle cannula tip into the skin of the animal.

In a specific embodiment, the invention encompasses a drug delivery device as disclosed in FIG. 22-FIG. 24 illustrate an example of a drug delivery device which can be used to practice the methods of the present invention for making intradermal injections illustrated in FIGS. 25-28. The device 10 illustrated in FIGS. 22-24. includes a needle assembly 20 which can be attached to a syringe barrel 60. Other forms of delivery devices may be used including pens of the types disclosed in U.S. Pat. No. 5,279,586, U.S. patent application Ser. No. 09/027,607 and PCT Application No. WO 00/09135, the disclosure of which are hereby incorporated by reference in their entirety.

The needle assembly 20 includes a hub 22 that supports a needle cannula 24. The limiter 26 receives at least a portion of the hub 22 so that the limiter 26 generally surrounds the needle cannula 24 as best seen in FIG. 22.

One end 30 of the hub 22 is able to be secured to a receiver 32 of a syringe. A variety of syringe types for containing the substance to be intradermally delivered according to the present invention can be used with a needle assembly designed, with several examples being given below. The opposite end of the hub 22 preferably includes extensions 34 that are nestingly received against abutment surfaces 36 within the limiter 26. A plurality of ribs 38 preferably are provided on the limiter 26 to provide structural integrity and to facilitate handling the needle assembly 20.

By appropriately designing the size of the components, a distance “d” between a forward end or tip 40 of the needle 24 and a skin engaging surface 42 on the limiter 26 can be tightly controlled. The distance “d” preferably is in a range from approximately 0.5 mm to approximately 3.0 mm, and most preferably around 1.5 mm±0.2 mm to 0.3 mm. When the forward end 40 of the needle cannula 24 extends beyond the skin engaging surface 42 a distance within that range, an intradermal injection is ensured because the needle is unable to penetrate any further than the typical dermis layer of an animal. Typically, the outer skin layer, epidermis, has a thickness between 50-200 microns, and the dermis, the inner and thicker layer of the skin, has a thickness between 1.5-3.5 mm. Below the dermis layer is subcutaneous tissue (also sometimes referred to as the hypodermis layer) and muscle tissue, in that order.

As can be best seen in FIG. 22, the limiter 26 includes an opening 44 through which the forward end 40 of the needle cannula 24 protrudes. The dimensional relationship between the opening 44 and the forward end 40 can be controlled depending on the requirements of a particular situation. In the illustrated embodiment, the skin engaging surface 42 is generally planar or flat and continuous to provide a stable placement of the needle assembly 20 against an animal's skin. Although not specifically illustrated, it may be advantageous to have the generally planar skin engaging surface 42 include either raised portions in the form of ribs or recessed portions in the form of grooves in order to enhance stability or facilitate attachment of a needle shield to the needle tip 40. Additionally, the ribs 38 along the sides of the limiter 26 may be extended beyond the plane of the skin engaging surface 42.

Regardless of the shape or contour of the skin engaging surface 42, the preferred embodiment includes enough generally planar or flat surface area that contacts the skin to facilitate stabilizing the injector relative to the subject's skin. In the most preferred arrangement, the skin engaging surface 42 facilitates maintaining the injector in a generally perpendicular orientation relative to the skin surface and facilitates the application of pressure against the skin during injection. Thus, in the preferred embodiment, the limiter has dimension or outside diameter of at least 5 mm. The major dimension will depend upon the application and packaging limitations, but a convenient diameter is less than 15 mm or more preferably 11-12 mm.

It is important to note that although FIGS. 22 and 23 illustrate a two-piece assembly where the hub 22 is made separate from the limiter 26, a device for use in connection with the invention is not limited to such an arrangement. Forming the hub 22 and limiter 26 integrally from a single piece of plastic material is an alternative to the example shown in FIGS. 22 and 23. Additionally, it is possible to adhesively or otherwise secure the hub 22 to the limiter 26 in the position illustrated in FIG. 24 so that the needle assembly 20 becomes a single piece unit upon assembly.

Having a hub 22 and limiter 26 provides the advantage of making an intradermal needle practical to manufacture. The preferred needle size is a small Gauge hypodermic needle, commonly known as a 30 Gauge or 31 Gauge needle. Having such a small diameter needle presents a challenge to make a needle short enough to prevent undue penetration beyond the dermis layer of an animal. The limiter 26 and the hub 22 facilitate utilizing a needle 24 that has an overall length that is much greater than the effective length of the needle, which penetrates the individual's tissue during an injection. With a needle assembly designed in accordance herewith, manufacturing is enhanced because larger length needles can be handled during the manufacturing and assembly processes while still obtaining the advantages of having a short needle for purposes of completing an intradermal injection.

FIG. 24 illustrates the needle assembly 20 secured to a drug container such as a syringe 60 to form the device 10. A generally cylindrical syringe body 62 can be made of plastic or glass as is known in the art. The syringe body 62 provides a reservoir 64 for containing the substance to be administered during an injection. A plunger rod 66 has a manual activation flange 68 at one end with a stopper 70 at an opposite end as known in the art. Manual movement of the plunger rod 66 through the reservoir 64 forces the substance within the reservoir 64 to be expelled out of the end 40 of the needle as desired.

The hub 22 can be secured to the syringe body 62 in a variety of known manners. In one example, an interference fit is provided between the interior of the hub 22 and the exterior of the outlet port portion 72 of the syringe body 62. In another example, a conventional Luer fit arrangement is provided to secure the hub 22 on the end of the syringe 60. As can be appreciated from FIG. 6, such needle assembly designed is readily adaptable to a wide variety of conventional syringe styles.

This invention provides an intradermal needle injector that is adaptable to be used with a variety of syringe types. Therefore, this invention provides the significant advantage of facilitating manufacture and assembly of intradermal needles on a mass production scale in an economical fashion.

Prior to inserting the needle cannula 24, an injection site upon the skin of the animal is selected and cleaned. Subsequent to selecting and cleaning the site, the forward end 40 of the needle cannula 24 is inserted into the skin of the animal at an angle of generally 90 degrees until the skin engaging surface 42 contacts the skin. The skin engaging surface 42 prevents the needle cannula 42 from passing through the dermis layer of the skin and injecting the substance into the subcutaneous layer.

While the needle cannula 42 is inserted into the skin, the substance is intradermally injected. The substance may be prefilled into the syringe 60, either substantially before and stored therein just prior to making the injection. Several variations of the method of performing the injection may be utilized depending upon individual preferences and syringe type. In any event, the penetration of the needle cannula 42 is most preferably no more than about 1.5 mm because the skin engaging surface 42 prevents any further penetration.

Also, during the administration of an intradermal injection, the forward end 40 of the needle cannula 42 is embedded in the dermis layer of the skin which results in a reasonable amount of back pressure during the injection of the substance. This back pressure could be on the order of 76 psi. In order to reach this pressure with a minimal amount of force having to be applied by the user to the plunger rod 66 of the syringe, a syringe barrel 60 with a small inside diameter is preferred such as 0.183″ (4.65 mm) or less. The method of this invention thus includes selecting a syringe for injection having an inside diameter of sufficient width to generate a force sufficient to overcome the back pressure of the dermis layer when the substance is expelled from the syringe to make the injection.

In addition, since intradermal injections are typically carried out with small volumes of the substance to be injected, i.e., on the order of no more than 0.5 ml, and preferably around 0.1 ml, a syringe barrel 60 with a small inside diameter is preferred to minimize dead space which could result in wasted substance captured between the stopper 70 and the shoulder of the syringe after the injection is completed. Also, because of the small volumes of substance, on the order of 0.1 ml, a syringe barrel with a small inside diameter is preferred to minimize air head space between the level of the substance and the stopper 70 during process of inserting the stopper. Further, the small inside diameter enhances the ability to inspect and visualize the volume of the substance within the barrel of the syringe.

As shown in FIGS. 22-24 the syringe 60 may be grasped with a first hand 112 and the plunger 66 depressed with the forefinger 114 of a second hand 116. Alternatively, as shown in FIGS. 8-10 the plunger 66 may be depressed by the thumb 118 of the second hand 116 while the syringe 60 is held by the first hand. In each of these variations, the skin of the animal is depressed, and stretched by the skin engaging surface 42 on the limiter 26. The skin is contacted by neither the first hand 112 nor the second hand 116.

An additional variation has proven effective for administering the intradermal injection of the present invention. This variation includes gripping the syringe 60 with the same hand that is used to depress the plunger 66. FIG. 22 shows the syringe 60 being gripped with the first hand 112 while the plunger is simultaneously depressed with the thumb 120 of the first hand 112. This variation includes stretching the skin with the second hand 114 while the injection is being made. Alternatively, as shown in FIG. 22, the grip is reversed and the plunger is depressed by the forefinger 122 of the first hand 112 while the skin is being stretched by the second hand 116. However, it is believed that this manual stretching of the skin is unnecessary and merely represents a variation out of habit from using the standard technique.

In each of the variations described above, the needle cannula 24 is inserted only about 1.5 mm into the skin of the animal. Subsequent to administering the injection, the needle cannula 24 is withdrawn from the skin and the syringe 60 and needle assembly 20 are disposed of in an appropriate manner. Each of the variations were utilized in clinical trials to determine the effectiveness of both the needle assembly 20 and the present method of administering the intradermal injection.

### 6. EXAMPLES

The following examples are illustrative, and should not be viewed as limiting the scope of the present invention. Reasonable variations, such as those that occur to reasonable artisan, can be made herein without departing from the scope of the present invention.

6.1 Dye Staining and Tracing in Vivo.

MATERIALS AND METHODS. In Vivo cell staining and all the following experiments were conducted under an approved IACUC protocol. Balb/c mice (Charles River Laboratories, Raleigh, N.C.), 6-8 weeks old, were anesthetized (IsoFlurane, Abbott Laboratories, Chicago, Ill.) and injected intradermally with 1% Evans Blue dye solution using a standard syringe with 34 gauge needle. The mice were dissected one hour post injection and the location of the dye observed. The mouse, as the human, has several main groups of easily identified draining lymph nodes.

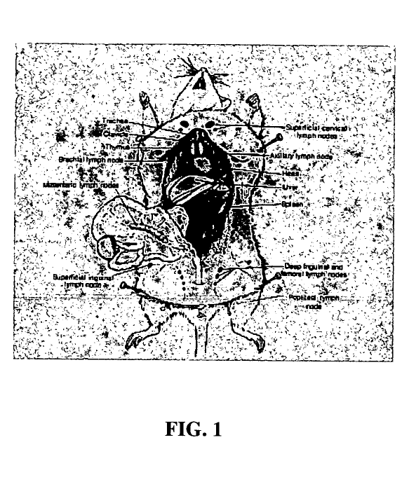

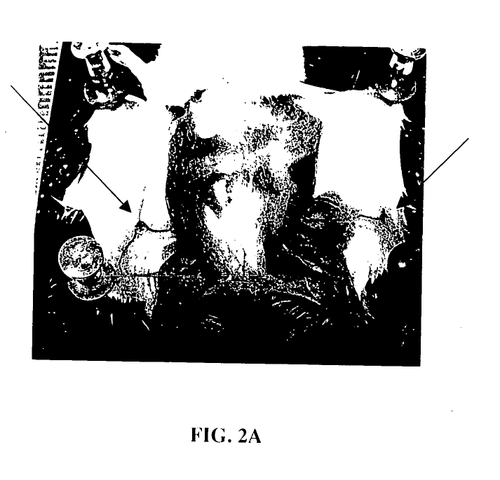

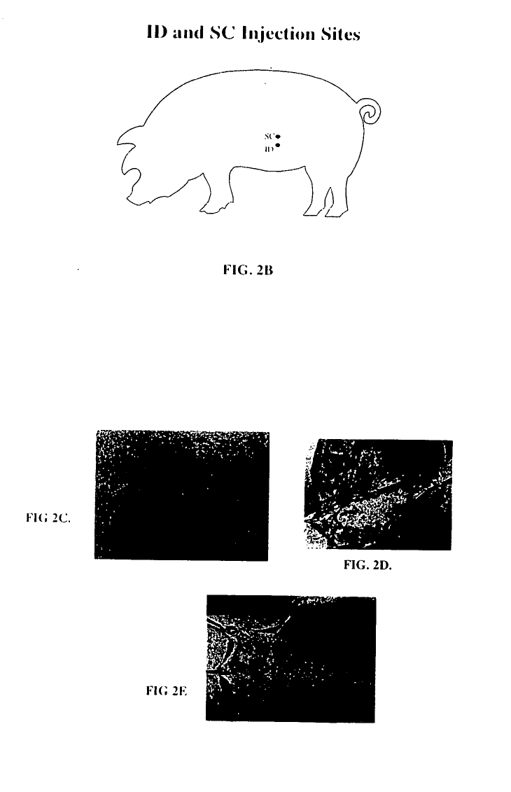

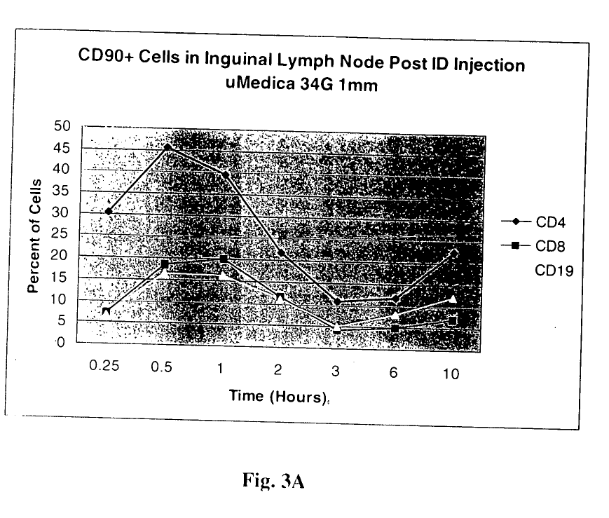

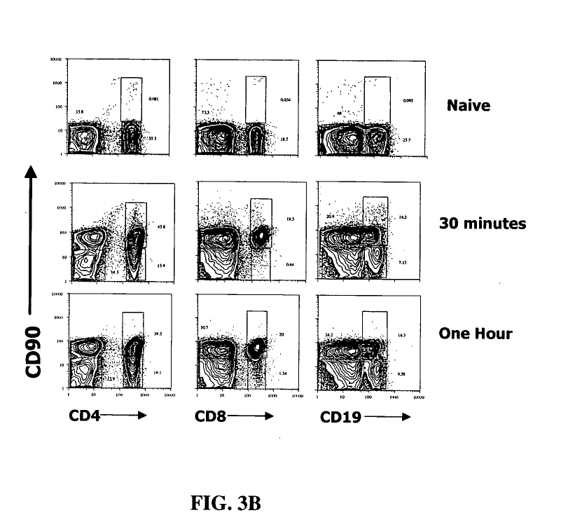

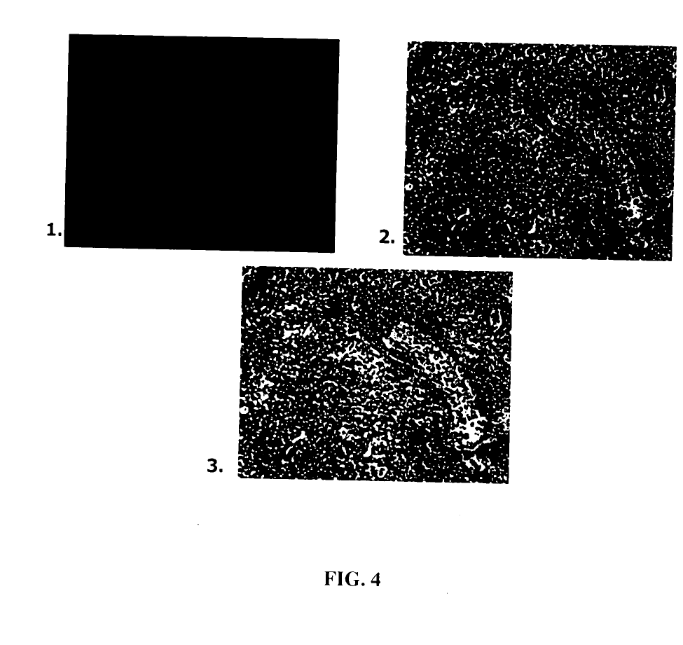

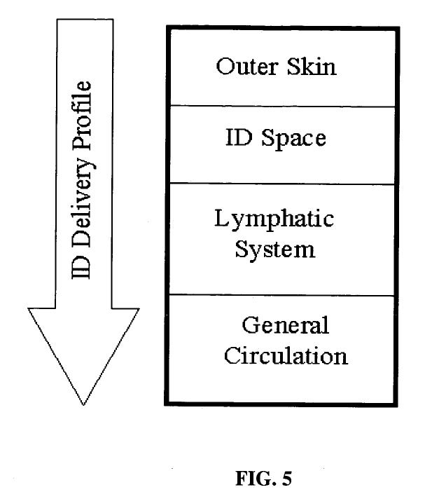

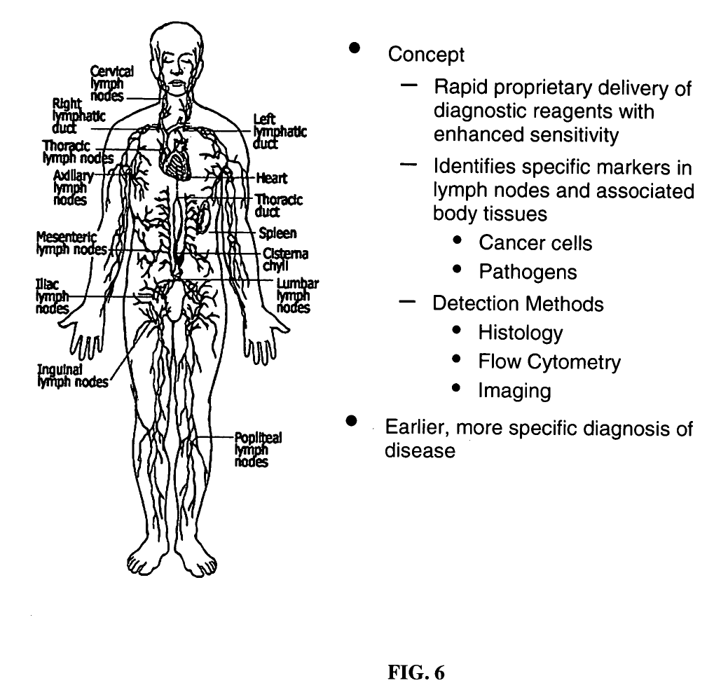

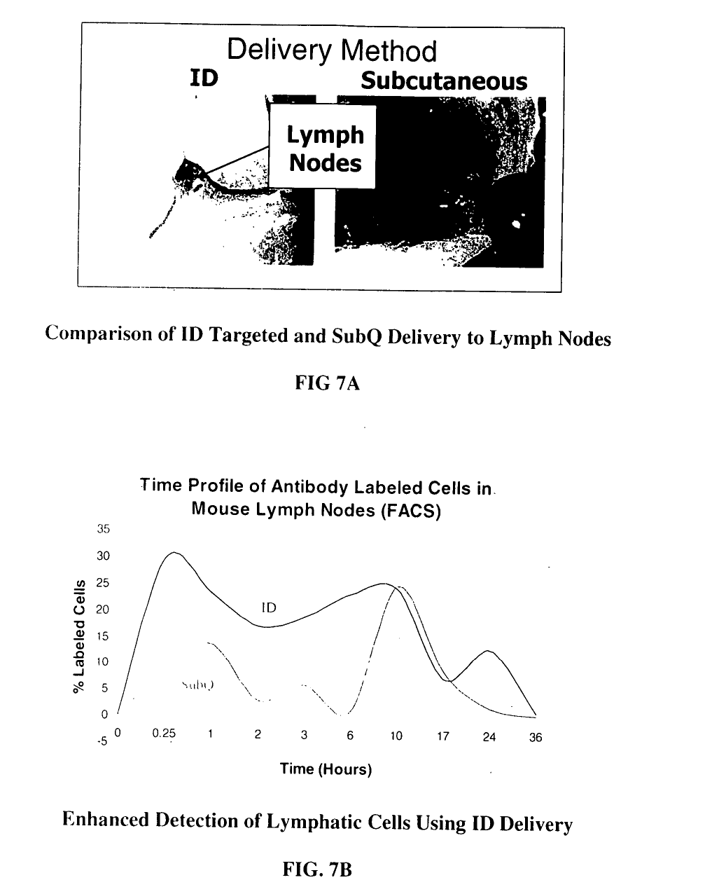

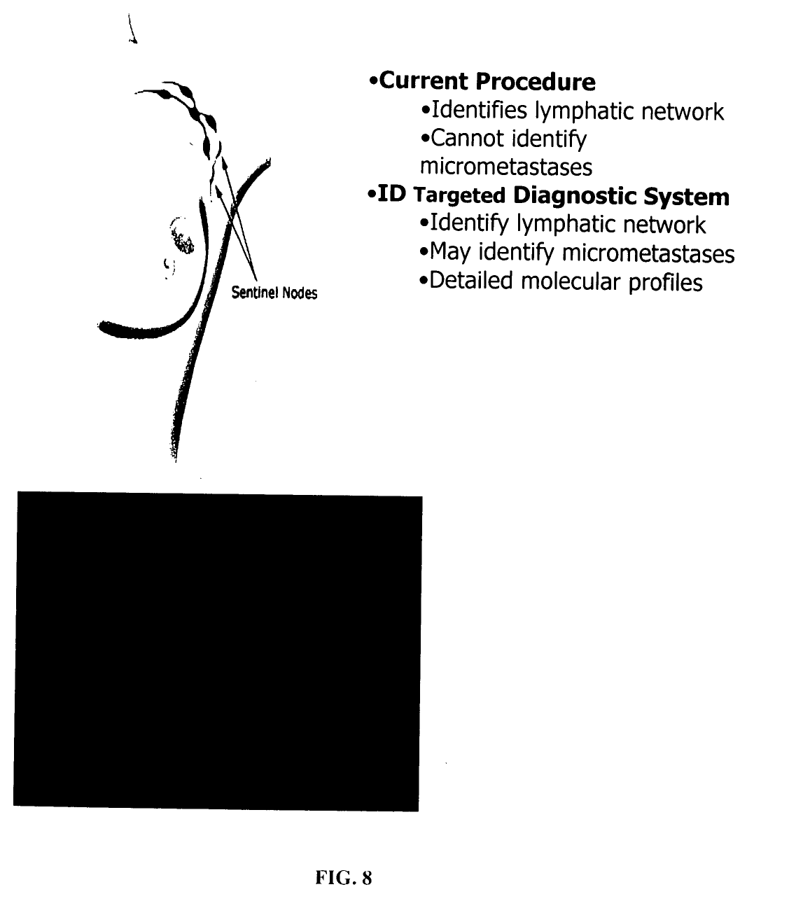

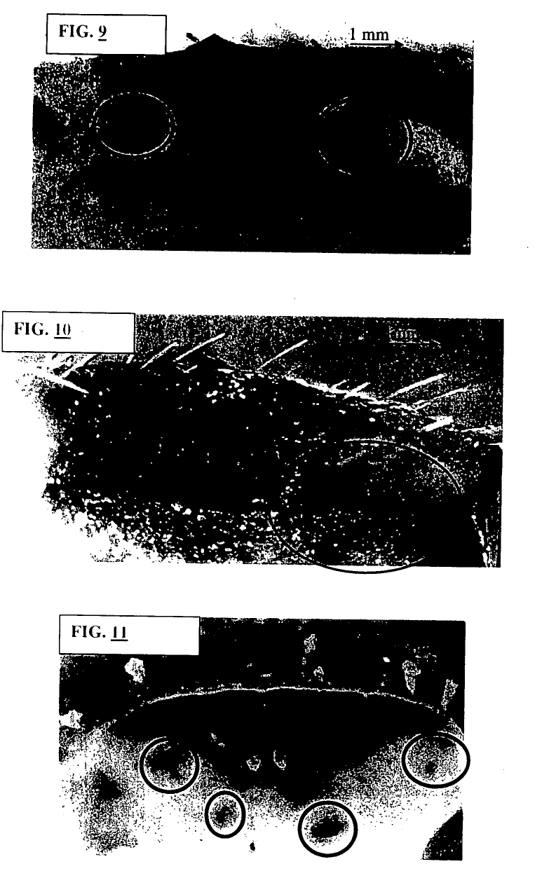

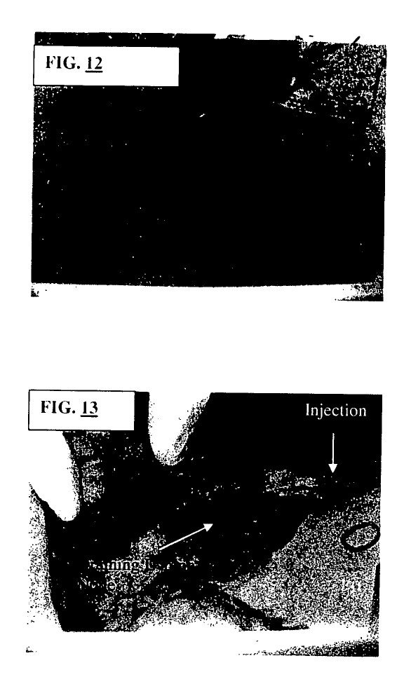

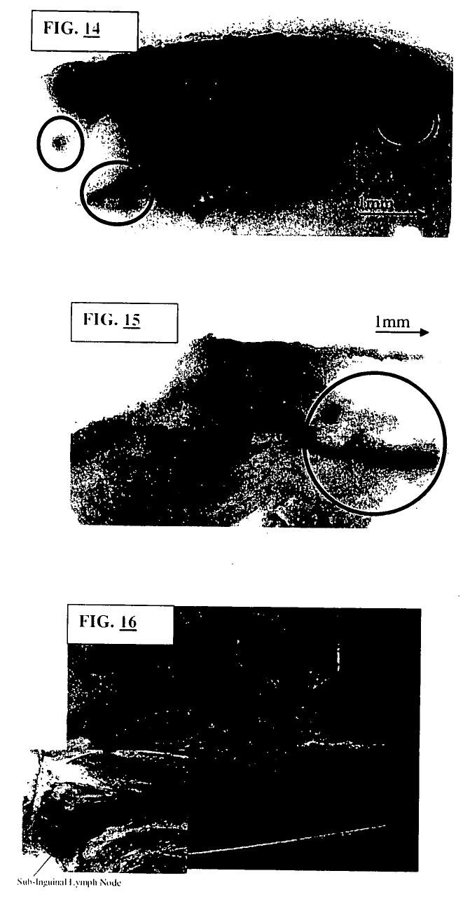

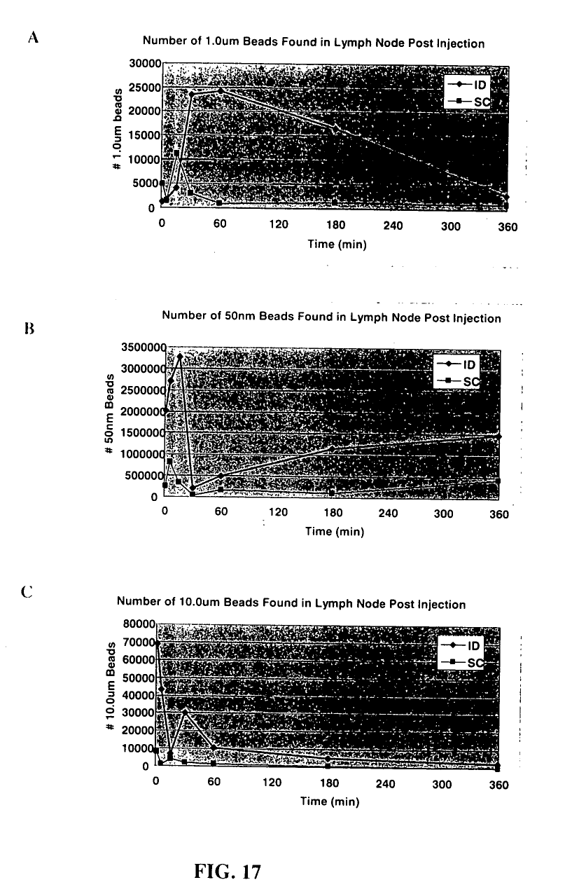

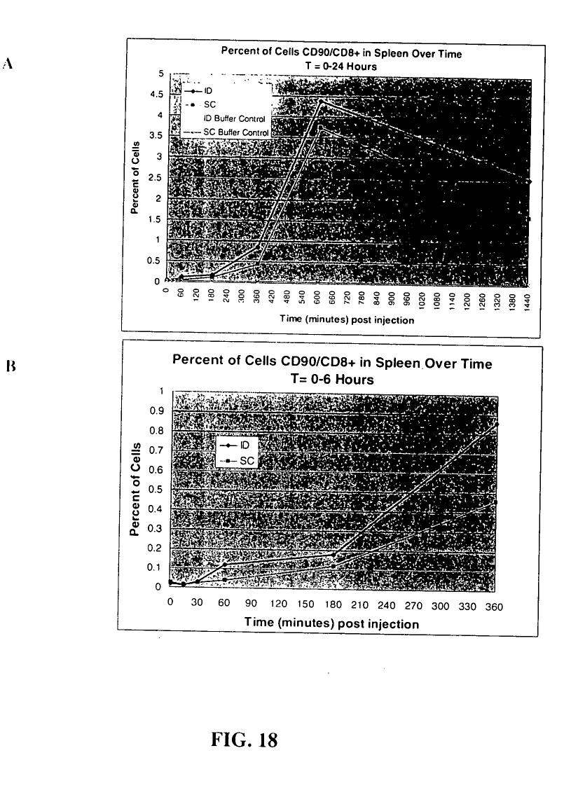

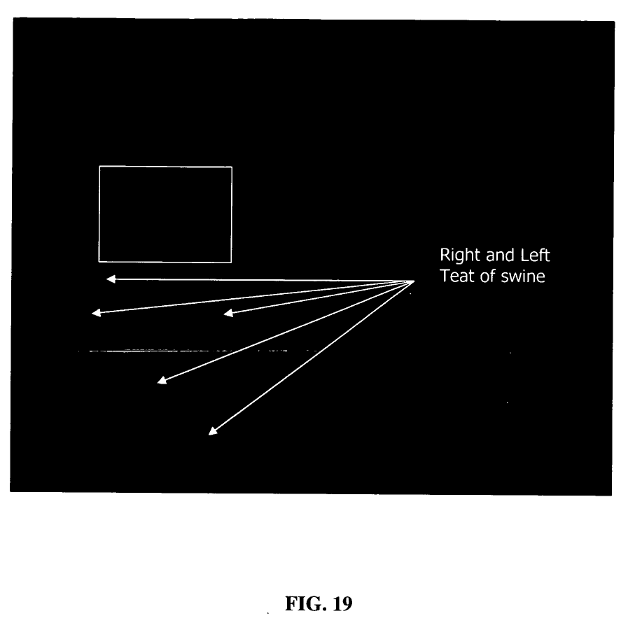

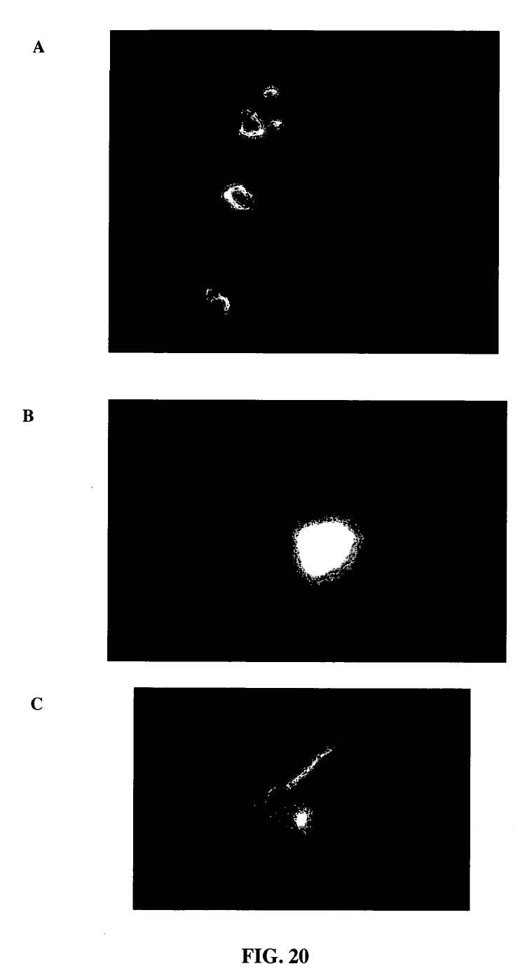

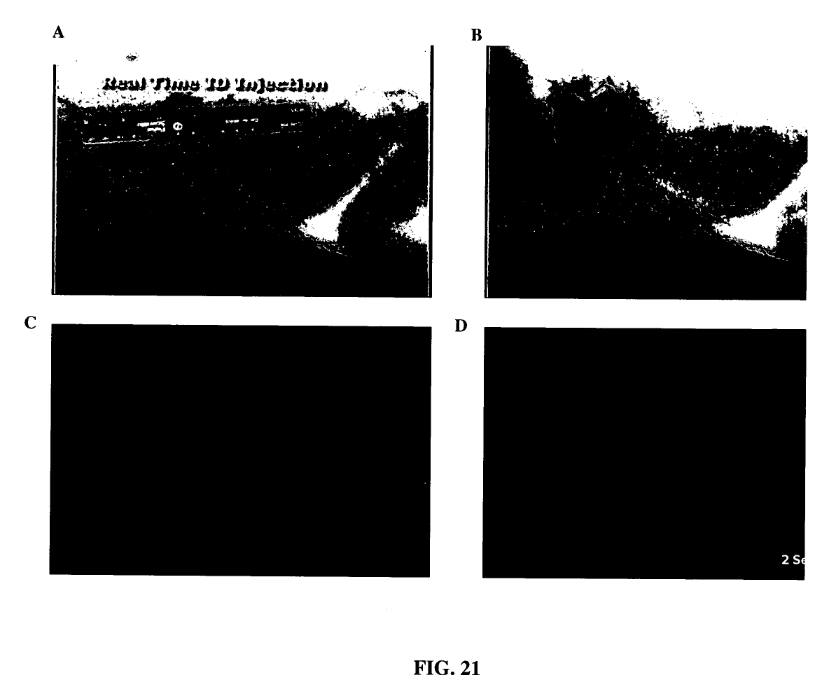

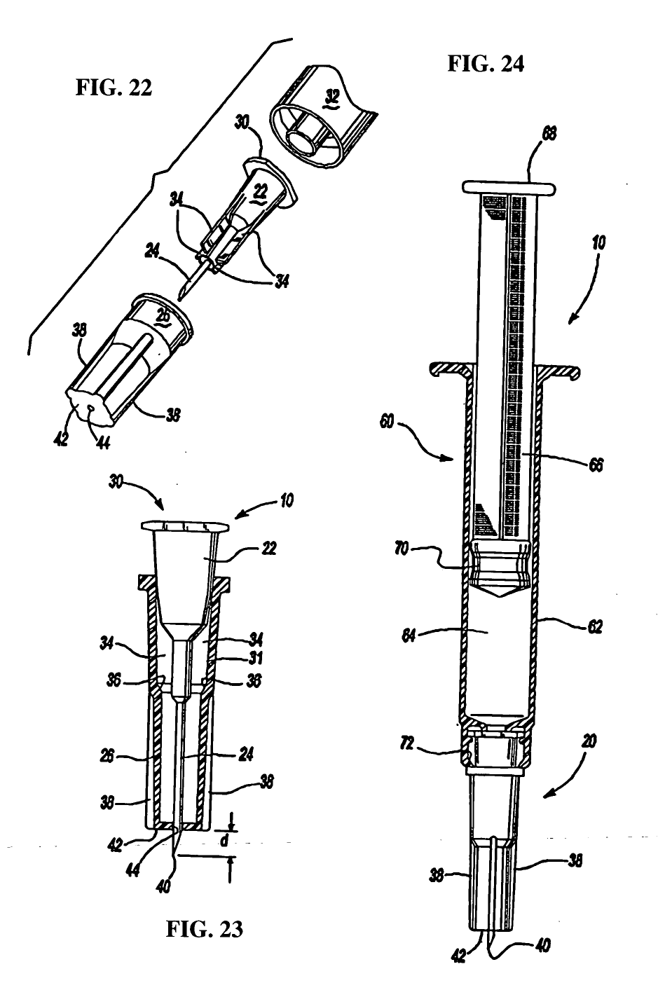

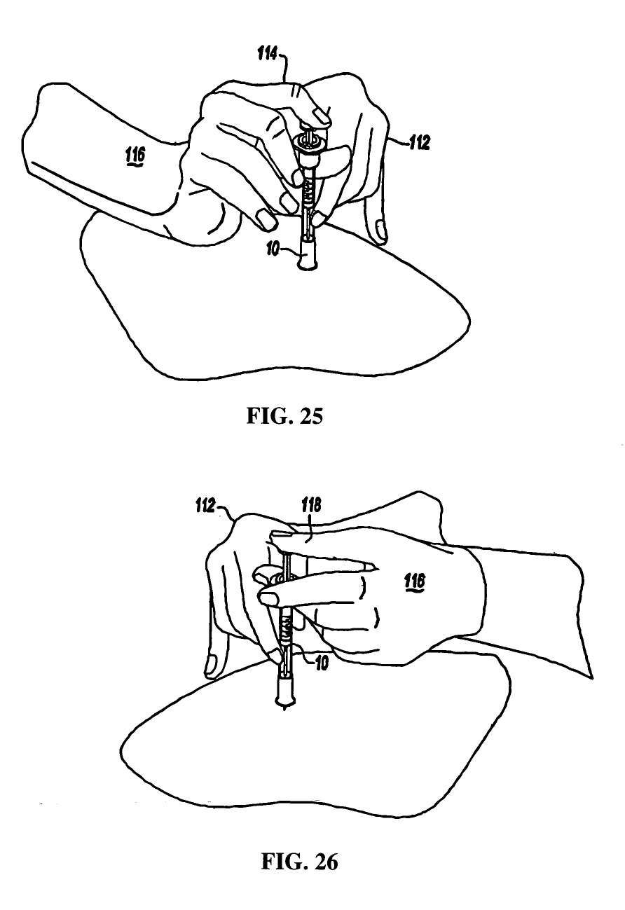

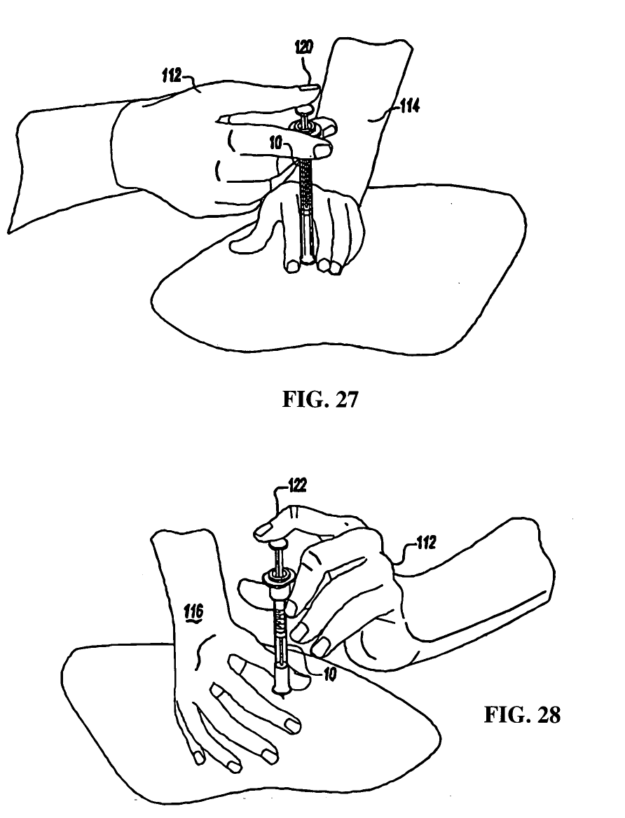

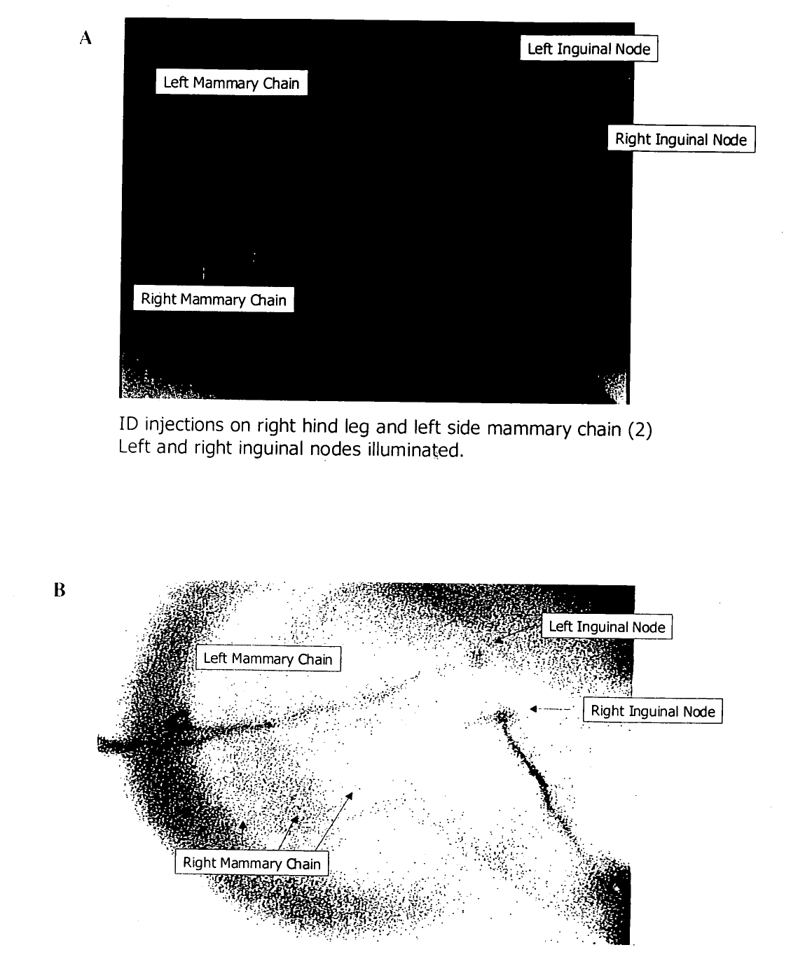

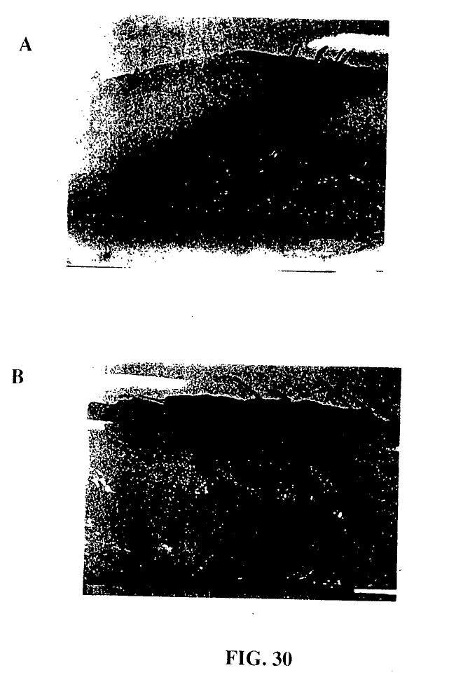

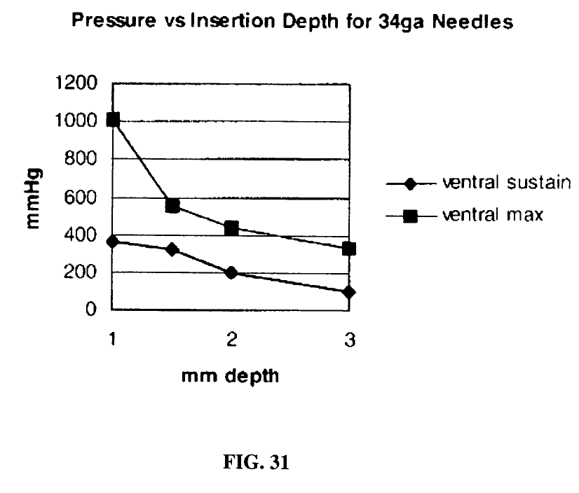

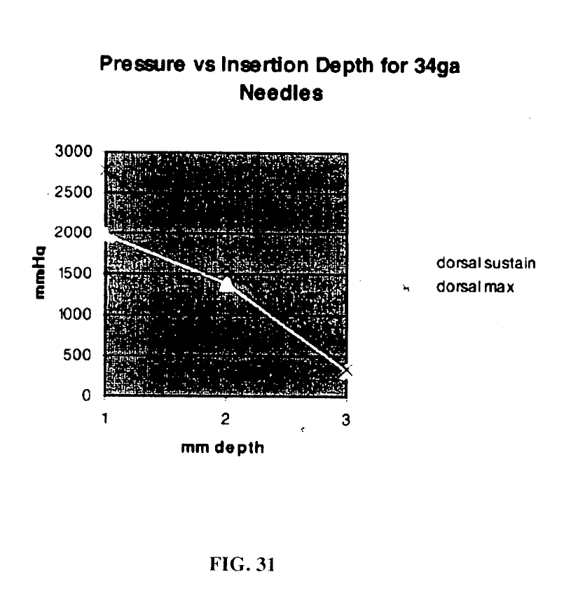

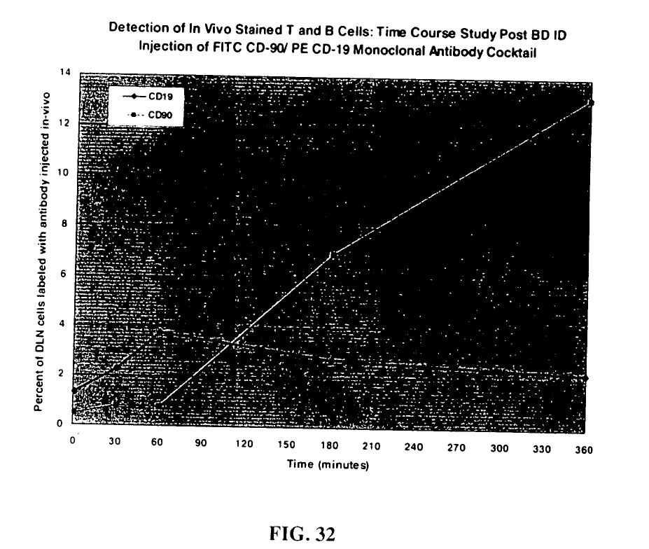

RESULTS. FIG. 1 illustrates the inguinal nodes that were targeted by the injection. FIG. 2 shows that the superficial inguinal lymph nodes were highly stained with the dye. The remaining dye at the injection site had not yet been trafficked to the lymph node. Therefore, one hour post injection, it was apparent that the dye had been transported to the lymph node as evidenced by the dark staining.

6.2 Dye Staining and Tracing in Vivo: Comparison of SC and ID Delivery. (Example 1A)

MATERIALS AND METHODS. In vivo cell staining and all the following experiments were conducted under an approved IACUC protocol. A Yorkshire swine (Charles River Laboratories, Raleigh, N.C.), 20-25 kg, was anesthetized (IsoFlurane, Abbott Laboratories, Chicago, Ill.) and injected with 1% Evans Blue dye solution (1) intradermally (ID) and perpendicular to the skin using a standard syringe with a 34-gauge needle 1 mm in length or (2) subcutaneously, at approximately a 30 degree angle, using a standard syringe with a 25 gauge/half inch (14 mm) needle (approximate depth of 7 mm). Injections were placed on the left dorsal side of the swine below the diaphragm and next to one another, as indicated in FIG. 2B. Visual observation made during ID injection noted immediate transport of the dye from the site of injection moving toward the regional draining lymph node, the inguinal node. This transport was visible through the skin and was extremely rapid, traversing tissues at velocities of up to 10 cm per second. Visual observation of the subcutaneous injected dye indicated no apparent transport of the dye. The swine was euthanized and dissected 10 minutes post injection and the location of the dye observed.

RESULTS. As evidenced in FIG. 2C to 2D, the ID injected dye moved rapidly through the lymphatic vasculature to the inguinal node while the subcutaneous injected dye remained at the site of injection and was not transported to the inguinal node. Therefore, it was apparent that ID injection was superior to subcutaneous for rapid targeted delivery of agents to the lymphatic system.

6.3 Antibody Staining and Flow Cytometry (Example 2)

MATERIALS AND METHODS. The model employed a fluorescein (FITC) labeled rat anti-CD90 (T cell marker) antibody. CD90 was a marker present on mature T lymphocyte cells. This reagent offered the opportunity to specifically label, in vivo, cells resident in lymph nodes. The antibody was introduced via a single bolus intradermal injection using a 34G 1 mm needle/catheter apparatus in the dorsal area. Trafficking of the antibody to the inguinal lymph nodes was monitored over time by flow cytometry and histological examination of relevant tissue sections (see Example 3).

Anesthetized Balb/c mice, 6-8 weeks old, as described above, were injected with a rat anti-CD90 (T cell marker) monoclonal antibody (clone 30-H12 Pharmingen, BD Biosciences, San Jose, Calif., specific for thymocytes, T lymphocytes and some dendritic cells) at lug/gram mouse as a single bolus intradermal injection using a 34G intradermal apparatus (needle/catheter configuration) in a total volume of 50 μL (20-25 μLs/lower side of dorsum of shaved mouse). At the appropriate time post injection the mice were sacrificed, and the inguinal lymph nodes and other appropriate tissues (spleen, thymus, kidney) were removed and prepared for flow cytometry analysis or histological examination (Example 3). Antibody amount could be as low as 5 ug/mouse.

For flow cytometry analysis, the tissue was placed in petri dishes containing 10 ml cold RPMI buffer (RPMI 1640, 5% FBS, 1% Pen/Strep, 0.5% β-mercaptoethanol, Invitrogen Life Technologies, Carlsbad, Calif.) for the lymph nodes, thymus, and kidneys. Spleens were placed in 10 ml cold red blood cell lysis buffer (0.16M NH₄Cl (Sigma, St. Louis, Mo.), 10 mM KHCO₃). Single cell suspensions were prepared by mashing the tissue through a 200μ mesh screen (VWR Scientific Products, West Chester, Pa.) under sterile conditions. Cell counts were taken using a 1:20 dilution from the resulting cell solution. Cells were centrifuged at 1500 rpm for 15 minutes at 4° C. Supernatant was aspirated and the cells were washed once with 5 mls RPMI buffer and centrifuged as earlier. Supernatant was aspirated and the cells were resuspended in Pharmingen stain buffer (Pharmingen, BD Biosciences, San Jose, Calif.) at 2-4×10⁸ cells/ml for flow staining. Approximately 1×10⁷ cells, 25 μL of the resuspended cells, was added to a well of a 96 well plate. Staining cocktail, 25 μL, was added to the cells in the well and mixed by pipetting. The cocktail consisted of the following labeled antibodies each at 0.01 mg/ml in Pharmingen Stain buffer, CY5PE-MAC1 (Caltag Laboratories, Burlingame, Calif.), CY5PE-GR1, APC-CD19, PE-CD4, APC-Cy7-CD8 (Pharmingen, BD Biosciences, San Jose, Calif.).

The cell/stain mix was incubated for 1 hour at 4° C. in the dark. The wells were washed with 150 uls FacsFlow buffer (Pharmingen) and centrifuged at 1500 rpm for 5 minutes at 4° C. The supernatant was aspirated and the wash was repeated. The washed cells were resuspended in 1 ml of cold FacsFlow buffer and kept on ice in the dark until analyzed by flow cytometry using a FACS Vantage SE. Cell analysis was gated for granulocytes and macrophages.

The lymph nodes were removed and the cells stained in vitro for analysis using the T cell markers CD4 and CD8 along with CD19 for B cell identification. The injected antibody contained the Fc region and binding to the Fc receptor on B cells was anticipated.

RESULTS. The results shown in FIGS. 3A and 3B were obtained via flow cytometry and indicate the rapid transport, in as little as 15 minutes, of the antibody from the intradermal compartment into the lymph node with subsequent binding to the CD90 molecule on the T cells and uptake by the B cells through the Fc receptor. FITC+ cells were observed at frequencies above 20% for up to 2 hours. The percentage of cells that bind the labeled antibody fluctuates over time as the circulating T cells flow into and then out of the lymph node. The antibody-labeled cells do not show up in the spleen until 6-10 hours post injection. Attempts at subcutaneous delivery of the labeled antibody met with confounded results as the tissue surrounding the lymph nodes contained high background signal from the antibody and was indistinguishable from specific node signal. General observations of the data indicate greater uptake and signal with intradermal delivery as compared to subcutaneous delivery.

6.4 Histological Examination. (Example 3)

MATERIALS AND METHODS. Tissues sections of the draining inguinal lymph nodes as obtained in Example 2 at each time point were examined by histological examination. The collected tissue was prepared as frozen sections in OCT media (Triangle Biomedical Sciences, Durham, N.C.). Samples were flash frozen on dry ice/2-methylbutane and then stored at −80° C. until sectioning. Tissues were serially sectioned at a depth of 12 microns and adhered to poly-L-lysine (Sigma) coated glass slides. The adhered tissue sections were hemolysin (Sigma) and eosin Y (Sigma) stained and mounted in VectaMount solution (Vector Laboratories, Burlingame, Calif.) and dried. Microscopic examination was conducted using a Nikon Eclipse TE300 confocal microscope.

RESULTS. The sections were stained with hematoxylin and eosin (H&E) and then microscopically examined. FIGS. 4A-4C showed the tissue from the lymph node of a mouse one hour after injection of the fluorescently labeled anti-CD90 antibody. As evidenced here, the injected fluorescent antibody did bind in vivo (FIG. 4A) to cells in the tissue indicating that it had maintained biological activity and signal. The mouse model showed successful targeted delivery of diagnostic reagents to the lymphatic system.

6.5 Administration of Dye at Various Depths, Volumes, and Rates in the Skin (Examples 4-9).

MATERIALS AND METHODS. The following experiments were conducted under an approved IACUC protocol. Yorkshire Swine (Charles River Laboratories, Raleigh, N.C.), approximately 20-25 kg, were anesthetized (Rompun 4 mg/kg, Xylazine 2 mg/kg, and Ketamine 2 mg/kg and maintained on 2% isoflurane) and injected intradermally with 1% Evans Blue (EB) dye solution at various volumes and needle penetration depths using a standard syringe and a 34 gauge needle. Injection volume and rate was controlled manually or with a Harvard Apparatus PhD 2000 programmable pump.

The skin at the injection site, including the injected material and surrounding tissue, was immediately excised after the injection. The tissue was flash frozen on dry ice/2-methylbutane and then stored at −80° C. until sectioning. The frozen tissue was cut longitudinally through the needle insertion point and immediately examined microscopically and photographed. Microscopic examinations were conducted using a Nikon SMZ-U dissecting scope with a Nikon FX-35PX 35 mm camera mount. Alternatively, after injection, the swine was euthanized and the tissue resected and photographed.

6.5.1 ID Administration of 50 uL with a 34G, 1.0 mm Needle at a Rate of 45 uL/min (Example 4)

One anesthetized Yorkshire swine was injected intradermally in the flank with 50 μL of EB through a 34G, 1.0 mm needle at a rate of 45 μL/min. A 2 cm² section around the injection site was excised and processed as described above. The results are shown in FIG. 9. The circled areas within the reticular dermis, separate from the main injection depot, show cross-sections of the draining lymphatic vessels (blue).

6.5.2 ID Administration of 100 uL with a 34 G, 1.0 mm Needle at a Rate of 45 uL/min (Example 5)

One anesthetized Yorkshire swine was injected interdermally in the flank with 100 μL of EB through a 34G, 1.0 mm needle at a rate of 45 μL/min. The skin sites were excised, flash frozen in methyl butane and cross-sectioned through the needle insertion point. The results are shown in FIGS. 10 and 11. In FIG. 10, the circled blue area within the reticular dermis, to the right of the main injection depot, shows a length-wise section of the draining lymphatic vessel. In contrast, a subpapillary capillary is shown within the same circle (red spot). In FIG. 11, the lymphatic vessels (blue spots) can be seen in at least five distinct areas around the injection depot.

6.5.3 ID Administration of 100 uL with a 34G, 1.0 mm Needle at a Rate of 100 uL/min (Example 6)

One anesthetized Yorkshire swine was injected in two sites interdermally in the flank with 100 μL of EB through a 34G, 1.0 mm needle at a rate of 100 μL/min. The depots were allowed to remain in the skin for 5 minutes before excision. The results are shown in FIGS. 12 and 13. In FIG. 12, the lymphatic vessels (blue) are clearly visible through the skin of the pig, leading away from the injection point (under the white gauze), towards the draining lymph node. In FIG. 13, surgical cut down confirms the drainage path seen previously in the excised tissue samples and in FIG. 12.

6.6 ID Administration of 100 uL with a 34G, 1.5 mm Needle at a Rate of 100 uL/min (Example 7)

One anesthetized Yorkshire swine was injected intradermally in the flank with 100 μL of EB through a 34 G, 1.5 mm needle at a rate of 100 μL/min. A 2 cm² section around the injection site was excised immediately following injection, flash frozen in methyl butane and cross-sectioned through the needle insertion point. As can be seen in FIG. 14, EB passes through the cannula and begins to generate pressure in the intradermal compartment. When sufficient pressure develops, the lymphatic vasculature opens and rapid transport of EB is sustained until EB delivery ceases. FIG. 14 shows an example of lymphatic vessels visible from a 1.5 mm injection (circled).

6.7 ID Administration of 50 uL with a 34G, 2 mm Needle at a Rate of 45 uL/min (Example 8)

One anesthetized Yorkshire swine was injected intradermally in the flank with 50 uL of EB through a 34G, 2 mm needle at a rate of 45 μL/min. A 2 cm² section around the injection site was excised immediately following injection, flash frozen in methyl butane and cross-sectioned through the needle insertion point. The results are shown in FIG. 15.

6.8 ID Administration of 200 uL with a a 34G, 1 mm Needle/Catheter (Example 9)

One anesthetized Yorkshire swine was manually injected intradermally above the right hoof with 200 uls of EB through a 34G, 1 mm needle/catheter. Within seconds, the dye traveled from the site of injection to the draining inguinal lymph node; the rate of travel could be visualized through the skin and approached 20 cm/second. Twenty minutes post injection the tissue was resected from the site of injection to the draining inguinal lymph nodes demonstrating long-range transport through lymphatic vasculature to deep tissues. The results are shown in FIG. 16.

6.9 Delivery of Beads to the Skin (Example 10): ID v. SC

The following example describes the advantages of intra-dermal delivery compared to subcutaneous delivery of agents for targeted delivery to the local lymphatic system.

MATERIALS AND METHODS. In Vivo particle injection and the following-experiments were conducted under an approved IACUC protocol. Balb/c mice (Charles River Laboratories, Raleigh, N.C.), 6-8 weeks old, 16-20 g, were anesthetized (IsoFlurane, Abbott Laboratories, Chicago, Ill.) and injected (1) mantoux style with fluoresecently labeled beads (Spherotech Inc., Libertyville, Ill.) 50 nm, 100 nm, 1 μm, or 10 μm in size as a single bolus dorsal intra-dermal injection using a 34 G, 1 mm length, intra-dermal apparatus (needle/catheter configuration) or (2) with a dorsal bolus subcutaneous injection using a 30 G needle, half inch/syringe apparatus in a total volume of 60 μls (30 μls/lower side of dorsum of shaved mouse). The number of beads delivered varied from size to size, however, all mice within each bead set received the same number of beads. At the appropriate time post injection the mice were sacrificed, and the inguinal lymph nodes were removed and prepared for flow cytometry analysis.

For flow cytometry analysis, the tissue was placed in petri dishes containing 10 ml cold sterile H₂O, in order to facilitate cell lysis. Single cell suspensions were prepared by mashing the tissue through a 200μ mesh screen (VWR Scientific Products, West Chester, Pa.) under sterile conditions creating a cell/bead suspension. The cell/bead suspension was centrifuged at 1500 rpm for 5 minutes at 4° C. Supernatant was aspirated and the pellet was resuspended in Pharmingen FacsFlow buffer (Pharmingen, BD Biosciences, San Jose, Calif.) and kept on ice in the dark until analyzed by flow cytometry using a FACS Vantage SE. Analysis was gated for fluorescent signal and the number of beads present in the sample counted.

RESULTS. Results as shown in FIG. 17 demonstrate improved bead delivery to the lymph node via intra-dermal delivery over subcutaneous injection for all bead sizes tested.

6.10 Comparison of ID vs SC Delivery of Specific Reagent to Spleen Tissue

Enclosed herein is an additional example of the benefits of targeted intradermal (ID) delivery. This example shows the improvement/enhancement of ID delivery of targeted reagents to the spleen compared to subcutaneous delivery.

Materials and Methods.

Animal Care: The following experiment was conducted under an approved IACUC protocol. Balb/c mice (Charles River Laboratories, Raleigh, N.C.) 6-8 weeks old, 16-20 g, were anesthetized (acepromozine, xylazine, ketamine) and injected intradermally (ID) (modified mantoux) using a standard syringe and a 34 gauge (34G), 1 mm needle/catheter or subcutaneously (SC) using a standard syringe and a 27 gauge needle.

Fluorescent antibody injections. Anesthetized Balb/c mice, 6-8 weeks old, were injected, as described above, with 20 ugs total, of a fluorescein isothiocyanate (FITC) labeled rat anti-CD90 (T cell marker) monoclonal antibody (clone 30-H12 Pharmingen, BD Biosciences, San Jose, Calif., specific for thymocytes, T lymphocytes and some dendritic cells) as a single bolus injection in a total volume of 50 μls (20-25 μls/lower side of dorsum of shaved mouse). At the appropriate time post injection the mice were sacrificed, and the spleen removed and prepared for flow cytometry analysis.

Flow Cytometry: For flow cytometry analysis, the tissue was placed in petri dishes containing 10 ml cold red blood cell lysis buffer (0.16M NH₄Cl (Sigma, St. Louis, Mo.), 10 mM KHCO₃). Single cell suspensions were prepared by mashing the tissue through a 200μ mesh screen (VWR Scientific Products, West Chester, Pa.) under sterile conditions. Cell counts were taken using a 1:20 dilution from the resulting cell solution. Cells were centrifuged at 1500 rpm for 15 minutes at 4° C. Supernatant was aspirated and the cells were washed once with 5 mls RPMI buffer and centrifuged as earlier. Supernatant was aspirated and the cells were resuspended in Pharmingen stain buffer (Pharmingen, BD Biosciences, San Jose, Calif.) at 2-4×10⁸ cells/ml for flow staining. Approximately 1×10⁷ cells, 25 μls of the resuspended cells, were added to a well of a 96 well plate. Staining cocktail, 25 μls, was added to the cells in the well and mixed by pipetting. The cocktail consisted of a combination of the following labeled antibodies, as appropriate, each at 0.01 mg/ml in Pharmingen Stain buffer, CY5PE-MAC1 (Caltag Laboratories, Burlingame, Calif.), CYSPE-GR 1, APC-CD19, PE-CD4, APC-Cy7-CD8 (Pharmingen, BD Biosciences, San Jose, Calif.). The cell/stain mix was incubated for 1 hour at 4° C. in the dark. The wells were washed with 150 uls FacsFlow buffer (Pharmingen) and centrifuged at 1500 rpm for 5 minutes at 4° C. The supernatant was aspirated and the wash was repeated. The washed cells were resuspended in 1 ml of cold FacsFlow buffer and kept on ice in the dark until analyzed by flow cytometry using a FACS Vantage SE. Cell analysis was gated for granulocytes and macrophages.

RESULTS: FIG. 18 shows the binding of the injected CD90-FITC antibody to T cells over time, post injection, in the spleen of mice. Initial appearance of the antibody in the spleen is 1 hour post injection. This delayed signal can be attributed to the heavy anesthesia used in the experiment. However, the percentage of cells labeled with the injected antibody was consistently higher in the ID injected mice than the SC injected mice indicating not only access to the spleen via ID injection but also greater tissue bio-availability.

6.11 Delivery of Cardio Green Imaging Agent(Indocyanine Green; “ICG”)

Enclosed herein is an additional example of the use of targeted intradermal (ID) delivery for the delivery of Cardiogreen (indocyanine green; “ICG”), an approved in vivo imaging agent for clinical use. This example shows the utility of targeted delivery as described in the patent mentioned above. This example complements the previous examples showing delivery of Evans Blue to swine and shows delivery of a near infrared (NIR) dye, lymphatic flow rate and dose sparing.

Materials and Methods

Animal Care: All experiments were conducted under an approved IACUC protocol. Yorkshire swine (Charles River Laboratories, Raleigh, N.C.), 20-25 kg, were anesthetized (telazol/xylazine/ketamine mix (35, 17.5, 17.5 mg/kg respectively, followed by continued isoflurane inhalation) and injected intradermally (90° angle) using a 34G, 1 mm needle/catheter and a standard syringe. IV injections were performed using a 27G, half-inch needle and delivered through a venous catheter. All recovered swine were intubated and hydrated throughout the procedure.

Dye Injections: Yorkshire swine were injected ID as described above, with 200 uls of 250 ug/ml indocyanine green (ICG) in sterile water (Fluka Chemical Corp., Milwaukee, Wis.). Injections sites included the right hind leg, and at the first and second teat of the left mammary chain. Additional injections of 200 μls and 75 μls were performed at 80 ug/ml indocyanine green in order to determine lymphatic flow rates. Intravenous injections were performed as described above with 5 mls of 2.5 mg/mL ICG.

Image Acquisition: Near infrared images were obtained using a tungsten lamp (Dolan-Jenner, Lawrence, Mass.) fitted with a 750 nm excitation filter (Omega Optical, Brattleboro, Vt.), a CCD camera (Kowa Co., Supercircuits CCTV camera model b/w Hi-Res ExVision) fitted with a 790 nm long pass emission filter (Omega Optical) and a Canon ZR-20 mini-DV camcorder. Images were acquired from the beginning of the injection until 40 minutes post injection. Images were processed using Adobe Premier v6.01 editing software. Speed of infusion through the lymphatic vessels determined from film footage.

RESULTS: As evidenced here, the images show that accurate targeting of lymphatic vasculature was achieved (FIGS. 19-21 and 29A and B). Lymphatic vessels and lymph nodes were easily visualized. Speed through the lymphatic vessels is effected by the volume injected, the rate of the infusion and the characteristics of the material infused. At a concentration of 80 ug/ml ICG, the speed through the lymphatic vessels was determined to be 5-10 cm/sec. In addition, dose-sparing effects were observed (FIGS. 20 and 21). An IV injection of 12.5 mgs of ICG, while illuminating the circulatory vasculature, did not illuminate the lymphatic vessels or any lymph nodes. An ID injection of 1000 fold less ICG, 6 and 16 ugs, did illuminate the lymphatic vasculature and draining inguinal lymph node. These results are indicative of improved sensitivity of ID delivery of imaging agents and as such indicate that further reduced amount of agent may be used to achieve the desired result when using advanced imaging techniques.

6.12 Comparison of ID and Mantoux Injections

Enclosed herein is an additional example of the benefits of targeted intradermal (ID) delivery. This example shows the improvement/enhancement of targeted delivery over the current Mantoux injection practice.

MATERIALS AND METHODS. The following experiment was conducted under an approved IACUC protocol. Yorkshire Swine (Charles River Laboratories, Raleigh, N.C.), approximately 20-25 kg, were anesthetized (Rompun 4 mg/kg, Xylazine 2 mg/kg, and Ketamine 2 mg/kg and maintained on 2% isoflurane) and injected intradermally with 1% Evans Blue (EB) dye solution using either a 34 G, 1 mm needle or a standard mantoux injection using a 27 G needle. Injection volume and rate was controlled manually or with a Harvard Apparatus PhD 2000 programmable pump.

The skin at the injection site, including the injected material and surrounding tissue, was immediately excised after the injection. The tissue was flash frozen on dry ice/2-methylbutane and then stored at −80° C. until sectioning. The frozen tissue was cut longitudinally through the needle insertion point and immediately examined microscopically and photographed. Microscopic examinations were conducted using a Nikon SMZ-U dissecting scope with a Nikon FX-35PX 35 mm camera mount.

Three anesthetized Yorkshire swine was injected intradermally in the flank with 25 uL of EB through either a 34G, 1.0 mm needle at a rate of 45 uL/min, or as a standard mantoux injection with a 27G needle. A 2 cm² section around the injection site was excised and processed as described above. Each injection was performed a total of three times. Measurements were taken of depot width, height and overall depth within the skin and a t-Test (Two-Sample Assuming Unequal Variances) was run on the data.

RESULTS. The average injection with the ID 34G, 1 mm needle had a significantly (p=0.05) smaller width than the Mantoux injection. The greater width within the SC compartment is expected due to the lower density of the tissue. Injections into the area tend to spread laterally just below the dermis following injection. There was no significant difference (p=0.45) between the overall heights of the injections. The depth within the tissue sample, however, did show a significant difference (p=0.03). The average lower depth of the 1 mm needle was 1.2 mm shallower than the Mantoux injection.

The result demonstrates the significant differences between Mantoux and 34G intradermal injections (see FIG. 30). The 34G 1 mm needles delivered compounds at shallower depths and more repeatedly into the ID compartment than can be accomplished through standard Mantoux methods.

6.13 Infusion Pressure Differences as a Function of Needle Insertion Depth and Tissue Environment

This example demonstrates the differences observed with ID delivery as a function of infusion pressure, needle insertion depth and tissue environment.

Materials and Methods:

Animal Care: All experiments were conducted under an approved IACUC protocol. Yorkshire swine (Charles River Laboratories, Raleigh, N.C.), 20-25 kg, were anesthetized (telazol/xylazine/ketamine mix (35, 17.5, 17.5 mg/kg respectively) and maintained on 2% isoflurane) and injected intradermally (90° angle) using a 34G depth limited 1.0, 1.5, 2.0, or 3.0 mm needle/catheter and a 500 μL Hamilton syringe. The catheter contained a WPI pressure gauge to measure injection pressure. Injection volume and rate was controlled with a Harvard Apparatus PhD 2000 programmable pump. All recovered swine were intubated and hydrated throughout the procedure.

An anesthetized swine was injected, as described above, with 100 uls of saline/injection at a rate of 100 uls/hour. Injections were performed both dorsally and ventrally. Multiple injections (4) with each needle configuration were conducted and pressure measurements recorded continuously throughout the injection.

RESULTS: FIGS. 31A and B depicts the maximum and average sustained pressures recorded as a function of needle depth. As shown in FIG. 31 delivery pressure during intradermal infusion depends on depth of penetration as controlled by needle length. Infusions using 1 and 1.5 mm needles have the highest pressure while 2 and 3 mm needles recorded lower pressures. It was observed that higher-pressure injections were accompanied by typical bleb formation (swelling and blanching of the skin) while lower pressure injections had reduced or absent blebs. A major contributing factor to these pressure differences is the deposition of fluid in the intradermal tissue versus the subcutaneous tissue. As the needle depth approaches and then reaches the subcutaneous tissue the infusion pressure decreases. The skin in the dorsal region was observed to provide more resistance to infusion than the ventral region; however, the trend in both regions was the same with decreasing resistance with increasing needle depth. Table 1 shows a summary of the Back pressure during in vivo intradermal infusion using various length 34ga needles.

TABLE 1
Back pressure during in vivo intradermal
infusion using various length 34ga needles.

| | pressure | | | |
|---|---|---|---|---|
| | (mmHg) | ventral | | |
| depth (mm) | ventral sustain | max | dorsal sustain | dorsal max |
| 1 | 367 | 1014 | 1997 | 2783 |
| 1.5 | 321 | 552 | | |
| 2 | 202 | 440 | 1372 | 1575 |
| 3 | 103 | 329 | 315 | 336 |

6.14 Delivery of a Cocktail of Antibodies to the Lymphatic System

This example shows ID delivery of a cocktail of monoclonal antibodies and their binding to the target cells in the draining lymph nodes. Also, the methods section is written to explain either the single monoclonal antibody injection of CD90-FITC, already in the patent, or the cocktail as delivered here.

Materials and Methods:

Animal Care: All experiments were conducted under an approved IACUC protocol. Balb/c mice (Charles River Laboratories, Raleigh, N.C.) 6-8 weeks old, 16-20 g, were anesthetized (Isoflurane, Abbott Laboratories, Chicago, Ill.) and injected intradermally (modified mantoux) using a standard syringe and a 34 gauge (34G), 1 mm needle/catheter.

Fluorescent antibody injections. Anesthetized Balb/c mice, 6-8 weeks old, were injected, as described above, with 20ugs total, of a fluorescein isothiocyanate (FITC) labeled rat anti-CD90 (T cell marker) monoclonal antibody (clone 30-H12 Pharmingen, BD Biosciences, San Jose, Calif., specific for thymocytes, T lymphocytes and some dendritic cells) or a combination of FITC-rat anti-CD90 and Phycoerythrin (PE) labeled rat anti-CD19 (B cell marker, clone 1D3 Pharmingen, BD Biosciences, San Jose, Calif. specific for B lymphocytes at all stages of development), monoclonal antibody cocktail (10:8 ug total respectively) as a single bolus intradermal injection using a 34G intradermal apparatus (needle/catheter configuration) in a total volume of 50 μls (20-25 μls/lower side of dorsum of shaved mouse). At the appropriate time post injection the mice were sacrificed, and the superficial inguinal lymph nodes and other appropriate tissues (spleen, thymus, kidney) were removed and prepared for flow cytometry analysis or histological examination.

Flow Cytometry: For flow cytometry analysis, the tissue was placed in petri dishes containing 10 ml cold RPMI buffer (RPMI 1640, 5% FBS, 1% Pen/Strep, 0.5% α-mercaptoethanol, Invitrogen Life Technologies, Carlsbad, Calif.) for the lymph nodes, thymus, and kidneys. Spleens were placed in 10 ml cold red blood cell lysis buffer (0.16M NH₄Cl (Sigma, St. Louis, Mo.), 10 mM KHCO₃). Single cell suspensions were prepared by mashing the tissue through a 200μ mesh screen (VWR Scientific Products, West Chester, Pa.) under sterile conditions. Cell counts were taken using a 1:20 dilution from the resulting cell solution. Cells were centrifuged at 1500 rpm for 15 minutes at 4° C. Supernatant was aspirated and the cells were washed once with 5 mls RPMI buffer and centrifuged as earlier. Supernatant was aspirated and the cells were resuspended in Pharmingen stain buffer (Pharmingen, BD Biosciences, San Jose, Calif.) at 2-4×10⁸ cells/ml for flow staining. Approximately 1×10⁷ cells, 25 μls of the resuspended cells, were added to a well of a 96 well plate. Staining cocktail, 25 μls, was added to the cells in the well and mixed by pipetting. The cocktail consisted of a combination of the following labeled antibodies, as appropriate, each at 0.01 mg/ml in Pharmingen Stain buffer, CY5PE-MAC1 (Caltag Laboratories, Burlingame, Calif.), CY5PE-GR1, APC-CD 19 (for CD90 only injected mice and controls), PE-CD4, APC-Cy7-CD8 (Pharmingen, BD Biosciences, San Jose, Calif.). Naïve mice were stained with the above labeled antibodies as well as FITC-CD90.

The cell/stain mix was incubated for 1 hour at 4° C. in the dark. The wells were washed with 150 uls FacsFlow buffer (Pharmingen) and centrifuged at 1500 rpm for 5 minutes at 4° C. The supernatant was aspirated and the wash was repeated. The washed cells were resuspended in 1 ml of cold FacsFlow buffer and kept on ice in the dark until analyzed by flow cytometry using a FACS Vantage SE. Cell analysis was gated for granulocytes and macrophages.

RESULTS: FIG. 32 demonstrates the in vivo labeling of both T and B cells in the draining lymph nodes of mice. In vitro staining controls indicated the available T and B cell population were 80 and 9 percent respectively. The conditions tested here did not stain all of the available cells in the lymph node, antibody concentrations were not optimized, but specific in vivo staining with a cocktail of monoclonal antibodies was observed.

1. A method for administration of at least one biologically active agent to a human subject, comprising delivering the agent into the intradermal compartment of the human subject's skin so that the agent has a higher tissue bioavailability in a particular tissue compared to when the same agent is delivered to a deeper tissue compartment.

2. The method of claim 1, wherein the deeper tissue compartment is subcutaneous compartment.

3. The method of claim 1, wherein the deeper tissue compartment is intramuscular compartment.

4. The method of claim 1 wherein about 10 pg to about 30 ng of the agent is accumulated in per 50 ug of the particular tissue.

5. The method of claim 1 wherein about 10 pg to about 15 ug of the agent is accumulated in per 50 ug of the particular tissue.

6. The method of claim 1 wherein about 1 cg to about 30 ng of the agent is accumulated in per 50 ug of the particular tissue.

7. A method for administration of at least one diagnostic agent to a human subject, comprising delivering the diagnostic agent into the intradermal compartment of the human subject's skin so that the diagnostic agent has a faster onset compared to when the same agent is delivered to the subcutaneous compartment.

8. A method for administration of at least one diagnostic agent to a particular tissue of a human subject, comprising delivering the diagnostic agent into the intradermal compartment of the human subject's skin so that the amount of the pre-selected dose of the diagnostic agent deposited in the particular tissue is increased compared to when the same agent is delivered to the subcutaneous compartment.

9. A method for administration of at least one diagnostic agent to a human subject, comprising delivering the agent into the intradermal compartment of the human subject's skin so that the agent has a higher tissue bioavailability compared to when the same agent is delivered by the ID Mantoux method.

10. A method for administration of at least one diagnostic agent to a human subject, comprising delivering the agent into the intradermal compartment of the human subject's skin so that the agent has a faster onset compared to when the same agent is delivered by the ID Mantoux method.

11. A method for administration of at least one diagnostic agent to a human subject, comprising delivering the agent into the intradermal compartment of the human subject's skin so that the amount of the pre-selected dose of the agent deposited in a particular tissue is increased compared to when the same agent is delivered by the ID Mantoux method.

12. A method for administration of at least one biologically active agent to a human subject, comprising delivering the agent into the intradermal compartment of the human subject's skin so that the agent is deposited in a particular tissue, wherein the agent specifically recognizes a cell which resides in the particular tissue.

13. A method for administration of a formulation comprising delivering the formulation into the intradermal compartment of the human subject's skin so that the formulation is deposited in a particular tissue, wherein the formulation comprises a first targeting agent and a second agent, so that the first targeting agent specifically recognizes a cell which resides in the particular tissue.

14. A method for administration of at least one diagnostic agent for the detection of a breast tumor to a human subject, comprising delivering the agent into the intradermal compartment of the human subject's skin at a controlled rate, volume, and pressure so that the agent is deposited in the intradermal compartment of the subject's skin.

15. A method for administration of at least one diagnostic agent for the detection of a breast tumor to a human subject, comprising delivering the agent into the intradermal compartment of the human subject's skin so that more than 75% of the pre-selected volume is deposited into the intradermal compartment, relative to when the same pre-selected volume is delivered to the intradermal compartment by the traditional ID Mantoux method.

16. A method for administration of at least one diagnostic agent for the detection of a breast tumor to a human subject, comprising delivering the agent into the intradermal compartment of the human subject's skin so that the agent is transported to the local lymphatic system.

17. A method for administration of at least one diagnostic agent for the detection of a breast tumor to a human subject, comprising delivering the agent into the intradermal compartment of the human subject's skin so that the agent has a higher tissue bioavailability compared to when the same agent is delivered by the ID Mantoux method.

18. A method for administration of at least one diagnostic agent for the detection of a breast tumor to a human subject, comprising delivering the agent into the intradermal compartment of the human subject's skin so that the agent has a faster onset compared to when the same agent is delivered by the ID Mantoux method.

19. A method for administration of at least one diagnostic agent for the detection of a breast tumor to a human subject, comprising delivering the agent into the intradermal compartment of the human subject's skin so that the amount of the pre-selected dose of the agent deposited in the lymphatic tissue is increased by at least 300% compared to when the same agent is delivered by the ID Mantoux method.

20. The method of any of claims 1, 7, 8, 9, and 10, wherein the particular tissue is selected from the group consisting of lymphatic tissue, mucosal tissue, lymph nodes, skin tissue, reproductive tissue, cervical tissue, vaginal tissue, lung, spleen, colon, thymus, bone marrow, Haemolymphoid tissue, and Lymphoid Tissue.

21. The method of claim 20, wherein the lymphoid tissue is selected from the group consisting of Epithelium-associated lymphoid Tissue, Mucosa-associated lymphoid Tissue, primary Lymphoid Tissue, and Secondary Lymphoid Tissue.

22. A method for administration of at least one diagnostic agent to a human subject, comprising delivering the agent at a pre-selected volume into the intradermal compartment of the human subject's skin so that more than 75% of the pre-selected volume is deposited into the intradermal compartment, relative to when the same pre-selected volume is delivered to the intradermal compartment by the traditional ID Mantoux method.

23. A method for administration of at least one diagnostic agent to a human subject, comprising delivering the agent into the intradermal compartment of the human subject's skin through a needle having a length sufficient to penetrate the intradermal compartment and an outlet at a depth within the intradermal compartment so that the agent is deposited into the intradermal compartment, wherein the needle is not inserted at a 15 degree angle so that at the site of deposition there is no elliptical wheal formation.

24. A method for administration of at least one biologically active agent to a particular tissue of a human subject, comprising delivering the agent into an intradermal compartment of the human subject's skin, wherein the agent is deposited in the particular tissue and specifically binds a marker of a disease in the particular tissue.

25. The method of claim 24, wherein the agent has a higher tissue bioavailability as compared to when the same agent is delivered to the subcutaneous compartment.

26. The method of claim 24, wherein the agent has a higher tissue bioavailability as compared to when the same agent is delivered by ID Mantoux method.

27. The method of claim 24, wherein the disease is a cancer, immune disease, an infectious disease, a disease of the lymphatic system, or a metabolic disease.

28. The method of claim 24, wherein the cancer is selected from the group consisting of lymphoma, leukemia, breast cancer, and colorectal cancer.

29. The method of any of claims 1, 7, 8, 9, and 10, wherein the agent is administered by a needle or a cannula.

30. The method of any of claims 1, 7, 8, 9, and 10, wherein the outlet of the needle or the cannula is inserted to a depth of about 300 um to about 3 mm.

31. The method of any of claims 1, 7, 8, 9, and 10, wherein the needle or cannula is 30-36 gauge.

32. The method of any of claims 1, 7, 8, 9, and 10, wherein the needle or cannula is 31-34 gauge.

33. The method of any of claims 1, 7, 8, 9, and 10, wherein the biologically active agent is selected from the group consisting of a peptide, a polypeptide, a protein, a nucleotide, a polynucleotide, a nucleic acid, a ligand for a receptor, an enzyme, a carbohydrate, a therapeutic agent, a chemospecific agent, antibody, monoclonal antibody, polyclonal antibody and an antibody fragment.

34. The method of claim 33, wherein the chemospecific agent is selected from the group consisting of a PNA, a photoaptamer, a sialic acid binder, a diboronic acid and a boronic acid.

35. The method of claim 24, wherein the particular tissue is on or surrounding a tumor cell in the particular tissue.

36. The method of claim 24, further comprising concurrently administering a tracer agent.

37. The method of claim 36, wherein the tracer agent is examiner in vivo and in real time.

38. The method of claim 36, wherein the tracer agent is examined in the subject ex vivo.

39. The method of claim 36, wherein the tracer agent is examined by flow cytometry.

40. The method of claim 36, wherein the tracer agent is examined by histological examination.

41. A method for diagnosis of a disease having a specific marker in a human subject comprising
(a) administering a biologically active agent into an intradermal compartment of a human subject's skin, wherein the agent is deposited in a particular tissue comprising the marker;
(b) tracing the agent;
(c) imaging the agent; and
(d) determining whether any specific binding of said agent occurs, wherein the presence of specific binding indicating a probability of said disease.

42. The method of claim 41, wherein the imaging agent is in vitro.

43. The method of claim 41, wherein the imaging agent is in vivo.

44. The method of claim 41, wherein the disease is selected from the group consisting of a cancer, immune disease, an infectious disease, a disease of the lymphatic system, or a metabolic disease.

45. The method of claim 41, further said imaging is performed by ultrasound, MRI, CT, PET, SPECT, X-ray, fluorescence, chemiluminescence, bioluminiscence, photoacoustic or optical methods.

46. The method of claim 41, wherein the imaging is obtained in real time.

47. The method of claim 41, wherein the imaging is obtained episodically.

48. The method of claim 41, further comprising administering a contrast agent.

49. The method of claim 41, wherein the contrast agent is selected from the group consisting of radiopaque materials, MRI imaging agents, ultrasound imaging agents, and optical imaging agents, so that the agent is suitable for the imaging method.

50. A method for administration of a formulation comprising at least one diagnostic agent to a human subject, comprising delivering the formulation into the intradermal compartment of the human subject's skin at a controlled rate, volume, and pressure so that the formulation is deposited in the intradermal compartment of the subject's skin

51. The method of claim 50, wherein the formulation comprises particles, and wherein the particles have a diameter of about 20 microns to about 1 nm.

52. The method of claim 50, wherein the particle is selected from the group consisting of liposomes, polymeric beads, particulate MRI contrast reagents, hollow particles, microbubbles, and microcrystalline beads.

53. The method of claim 50, wherein the formulation comprises nanoparticles, and wherein the nanoparticles have a diameter of about 1 nm to about 20 microns.

54. The method of claim 50, wherein the concentration of the at least one diagnostic agent is about 10 mg/mL.

55. The method of claim 50, wherein the concentration of the at least one diagnostic agent is about 100 mg/mL.

56. The method of claim 50, wherein the concentration of the at least one diagnostic agent is between about 20 ug/mL to 100 mg/mL.

57. The method of claim 50, wherein the amount of the at least one diagnostic agent delivered is between about 5 and 10 ug.

58. The method of claim 50, wherein the formulation comprises at least one additional molecule selected from the group consisting of a therapeutic agent, a tracer, an excipient, an additive, a chemospecific agent, and a marker.

59. A method for administration of at least one biologically active agent to a human subject, comprising delivering the agent into the intradermal compartment of the human subject's skin so that the agent specifically binds a biological entity.

60. The method of claim 59, wherein the biologically active agent is a diagnostic agent.

61. The method of claim 52, wherein the biological entity is selected from the group consisting of a cell, group or collection of cells, a bacteria, a virus, a pathogen, a protein, a plaque, and a parasitic agent.
